(19) United States
(12) Patent Application Publication
(10) Pub. No.: US 2025/0197857 A1
(43) Pub. Date: Jun. 19, 2025

(54) OLIGONUCLEOTIDE COMPOSITIONS AND METHODS THEREOF FOR EXON SKIPPING

(71) Applicant: WAVE LIFE SCIENCES LTD., Singapore (SG)

(72) Inventors: Xiao Shelley Hu, Brookline, MA (US); Stephen Lister Lake, Newton, MA (US); Michael Angelo Panzara, Boston, MA (US); Sarah Diane Lamore, Burlington, MA (US); Andrew Harrison Hart, Litchfield, NH (US); Danlin Xu, Summit, NJ (US); Keith Andrew Bowman, Stow, MA (US); Mamoru Shimizu, Arlington, MA (US); Vincent Aduda, Acton, MA (US); Mohammed Rowshon Alam, Hopkinton, MA (US); Philip Ross, Milford, MA (US); Jianxin Gao, Norton, MA (US); Yilin Zhang, West Roxbury, MA (US)

(21) Appl. No.: 18/843,171

(22) PCT Filed: Mar. 2, 2023
(86) PCT No.: PCT/US2023/014401

§ 371 Date: Aug. 30, 2024

Related U.S. Application Data
(60) Provisional application No. 63/433,733, filed on Dec. 19, 2022.
(60) Provisional application No. 63/424,418, filed on Nov. 10, 2022.
(60) Provisional application No. 63/397,221, filed on Aug. 11, 2022.
(60) Provisional application No. 63/315,952, filed on Mar. 2, 2022.

(51) Int. Cl. C12N 15/113; A61P 21/00

(52) U.S. Cl. CPC C12N 15/113; A61P 21/00; C12N 2310/315; C12N 2310/321; C12N 2310/322; C12N 2320/35

(57) ABSTRACT

Among other things, the present disclosure provides various technologies including chirally controlled oligonucleotide compositions and technologies for manufacturing and using such oligonucleotide compositions. In some embodiments, the present disclosure provides technologies useful for preventing or treating various conditions, disorders or diseases, e.g., Duchenne Muscular Dystrophy.

### CROSS-REFERENCE TO RELATED APPLICATIONS

This application claims priority to United States Provisional Application Nos. 63/315,952 filed Mar. 2, 2022, 63/397,221 filed Aug. 11, 2022, 63/424,418 filed Nov. 10, 2022, and 63/433,733 filed Dec. 19, 2022, the entirety of each of which is incorporated herein by reference.

### BACKGROUND

Muscular dystrophy (MD) is a group of muscle conditions, diseases, or disorders that is reported to result in (increasing) weakening and breakdown of skeletal muscles over time. The conditions, diseases, or disorders differ in which muscles are primarily affected, the degree of weakness, when symptoms begin, and how quickly symptoms worsen. Many MD subjects eventually become unable to walk. In many cases muscular dystrophy is fatal. Some types are also associated with problems in other organs, including the central nervous system. In some embodiments, a muscular dystrophy is Duchenne (Duchenne's) Muscular Dystrophy (DMD). In some embodiments, a muscular dystrophy is Becker (Becker's) Muscular Dystrophy (BMD)

### SUMMARY

While the US Food and Drug Administration (FDA) has approved treatments for DMD including oligonucleotides golodirsen and viltolarsen, there remains an unmet need for more technologies to treat muscular dystrophy including DMD. Among other things, at the time of the present invention clinical benefits of the approved oligonucleotide drugs have not been established.

WVE-N531 is an oligonucleotide compound that has demonstrated exon skipping activity and DMD function restoration in vitro and in mouse models. Structurally, WVE-N531 belongs to a different type of oligonucleotide than golodirsen and viltolarsen. For example, both golodirsen and viltolarsen are phosphorodiamidate morpholino oligomers (PMOs), while WVE-N531 is a 2′-F and 2′-OMe modified oligonucleotide in which the internucleotidic linkages are independently phosphorothioate, n001 and natural phosphate linkages. Further, chiral linkage phosphorus centers are not stereodefined, and both golodirsen and viltolarsen are provided and administered as stereorandom compositions in which their chiral linkage phosphorus centers randomly exist in two of the potential configurations (Rp or Sp): the compositions are a random mixture of more than 33 million (2²⁵) and 1 million (2²⁰) diastereomers for golodirsen and viltolarsen, respectively. In contrast, chiral linkage phosphorus in WVE-N531 is each independently stereodefined: linkage phosphorus of each phosphorothioate linkage is Sp, and linkage phosphorus of each n001 linkage is Rp. As demonstrated herein, WVE-N531 compositions are chirally controlled compositions in which WVE-N531 or salts thereof are highly enriched over other diastereomers or salts thereof. Still further, to Applicant's knowledge prior to the present disclosure oligonucleotides comprising phosphoryl guanidine linkages such as n001 have not been systematically administered to and assessed in human subjects for therapeutic uses. An oligonucleotide, suvodirsen, comprises certain chemistry moieties such as 2′-F, 2′-OMe, phosphorothioate linkages, etc. that are more similar to WVE-N531 than golodirsen and viltolarsen and was also manufactured and administered as a chirally controlled oligonucleotide composition, but it failed in human clinical trials at the assessed doses including about 4.5 mg/kg.

Among other things, the present disclosure provides technologies for treating muscular dystrophy such as DMD. In some embodiments, the present disclosure provides doses and dosage regimens that are sufficiently safe for clinical administration to subjects and are effective to provide DMD exon 53 skipping, production of truncated DMD polypeptide that can perform improved levels of one or more functions of wild-type DMD protein compared to absence of WVE-N531, DMD function restoration or improvement, and/or clinical benefits. In some embodiments, as demonstrated herein WVE-N531 can provide improved properties and/or activities, e.g., AUC, Cₘₐₓ, tissue distribution, and/or plasma half life. compared to suvodirsen in primate subjects including human. Various results presented herein support that WVE-N531 can be safely and effectively utilized to treat conditions, disorders or diseases associated with DMD mutations that are amenable to exon 53 skipping including in human subjects.

In some embodiments, the present disclosure provides a method for treating muscular dystrophy, e.g., DMD, comprising administering to a subject suffering therefrom WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg (mg per kg of body weight) WVE-N531 free acid form, wherein the subject has a mutation of the DMD gene that is amenable to exon 53 skipping. In some embodiments, WVE-N531 is administered as a pharmaceutically acceptable salt form. In some embodiments, WVE-N531 is administered as a WVE-N531 hexadecasodium salt form. In some embodiments, WVE-N531 is administered as one or more forms including one or more pharmaceutically acceptable salt forms, and the total amount of all WVE-N531 forms is equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg of the free acid form. In some embodiments, WVE-N531 is administered in a pharmaceutical composition.

In some embodiments, the present disclosure provides a method for treating muscular dystrophy, e.g., DMD, comprising administering to a subject suffering therefrom a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the subject has a mutation of the DMD gene that is amenable to exon 53 skipping. In some embodiments, a pharmaceutically acceptable carrier is or comprises a buffered solution. In some embodiments, it is a phosphate buffered solution. In some embodiments, WVE-N531 is dissolved in a solution and exist in one or more forms, wherein the total amount of all WVE-N531 forms is equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg of the free acid form.

In some embodiments, the present disclosure provides a method for providing DMD exon 53 skipping in a subject, comprising administering to the subject WVE-N531, or a pharmaceutical composition comprising WVE-N531 and a pharmaceutically acceptable carrier, at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, the present disclosure provides a method for restoring DMD RNA reading frame in a subject, comprising administering to the subject WVE-N531, or a pharmaceutical composition comprising WVE-N531 and a pharmaceutically acceptable carrier, at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, the present disclosure provides a method for providing a truncated DMD polypeptide in a subject, comprising administering to the subject WVE-N531, or a pharmaceutical composition comprising WVE-N531 and a pharmaceutically acceptable carrier, at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, a truncated (compared to wild-type DMD protein) DMD polypeptide is encoded by an exon53-skipped DMD mRNA. In some embodiments, such an mRNA may lack one or more additional exons in addition to exon 53. In some embodiments, such a truncated DMD polypeptide provides one or more functions, partially or fully, of a wild-type DMD protein. In some embodiments, the present disclosure provides a method for providing increased level of a DMD function in a subject, comprising administering to the subject WVE-N531, or a pharmaceutical composition comprising WVE-N531 and a pharmaceutically acceptable carrier, at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, a subject has a mutation in the DMD gene that is amenable to exon 53 skipping. In some embodiments, a subject is suffering from or susceptible to a muscular dystrophy, e.g., DMD.

In some embodiments, two or more doses are administered, each independently of an amount as described herein, e.g., equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, about 2-10 doses are administered. In some embodiments, 10 or more doses are administered. In some embodiments, each dose is about the same.

In some embodiments, two or more (e.g., about 2, 3, 4, 5, 6, 7, 8, 9, or 10 or more) doses are administered about weekly. In some embodiments, two or more (e.g., about 2, 3, 4, 5, 6, 7, 8, 9, or 10 or more) doses are administered about every two weeks. In some embodiments, two or more (e.g., about 2, 3, 4, 5, 6, 7, 8, 9, or 10 or more) doses are administered about every 3, 4, 5, 6, 7, 8, 9 or 10 weeks. In some embodiments, doses are administered about weekly. In some embodiments, doses are administered about every two weeks. In some embodiments, doses are administered about every three weeks. In some embodiments, doses are administered about every four weeks. In some embodiments, doses are administered about monthly. In some embodiments, doses are administered about every 5, 6, 7, 8, 9, or 10 weeks. In some embodiments, three or more doses are administered about every two weeks. In some embodiments, doses are first administered with shorter intervals (e.g., about every two weeks) and then administered with longer intervals (e.g., about every 3, 4, 5, 6, 7, 8, 9 or 10 weeks). In some embodiments, two or more doses, e.g., about 2, 3, 4, 5, 6, 7, 8, 9, or 10 doses, are administered about every two weeks, followed by one or more doses each independently administered about 3, 4, 5, 6, 7, 8, 9 or 10 weeks from its previous doses. In some embodiments, about 2, 3, 4, 5, 6, 7, 8, 9, or 10 doses are administered about every two weeks, and one or more doses are administered about every 3, 4, 5, 6, 7, 8, 9 or 10 weeks, or about monthly. In some embodiments, doses are administered about every two weeks for about 2, 3, 4, 5, 6, 7, 8, 9, 10, 11, 12, 13, 14, 15, or 16 or more weeks from first dose and then doses are administered about every 3, 4, 5, 6, 7, 8, 9 or 10 weeks. In some embodiments, doses are administered every two weeks for about 4, 8, 12, or 16 or more weeks from first dose and then doses are administered about every 4 weeks. In some embodiments, doses are administered every two weeks for about 4, 8, 12, or 16 or more weeks from first dose and then doses are administered about monthly. In some embodiments, doses are first administered about every two weeks, and then after about 4 weeks, 8 weeks, 12 weeks, or 16 weeks from first dose, doses are administered about every four weeks or about monthly. In some embodiments, each dose is independently about the same, e.g., about 10 mg/kg.

In some embodiments, WVE-N531 or a composition thereof is administered intravenously. In some embodiments, WVE-N531 or a composition thereof is administered intramuscularly. In some embodiments, a dose is administered as an about 20-60, e.g., about 20, 25, 30, 35, 40, 45, 50, 55 or 60, minutes intravenous infusion. In some embodiments, infusion is administered with about 1, 2, 3, or 4 hours. In some embodiments, if a dose is missed, it may be administered as soon as possible after the scheduled dose.

In some embodiments, a subject is a pediatric subject. In some embodiments, a subject is boy subject. In some embodiments, a subject is no younger than 5 years old. In some embodiments, a subject is no older than 18 years old. In some embodiments, a subject meets one or more or all the inclusion criteria described in the Examples. In some embodiments, a subject is selected based on one or more or all the inclusion and exclusion criteria described in the Examples.

In some embodiments, WVE-N531 is administered at a dose that can achieve a clinically significant maximum plasma concentration.

In some embodiments, a subject is administered a steroid at least about six months (or 24 weeks) prior to the first dose of WVE-N531 or a composition thereof. In some embodiments, a subject is administered a corticosteroid at least about six months (or 24 weeks) prior to the first dose of WVE-N531 or a composition thereof. In some embodiments, a subject is on a stable corticosteroid therapy regimen. In some embodiments, a corticosteroid is deflazacort. In some embodiments, a subject is administered a steroid at least about one month prior to the first dose of WVE-N531 or a composition thereof.

In some embodiments, provided technologies (oligonucleotides, compositions, methods, doses, dosage regimens, etc.) provide one or more desired biological effects. In some embodiments, provided technologies provide exon 53 skipping. In some embodiments, provided technologies provide exon 53 skipping at clinically significant levels. In some embodiments, provided technologies increase levels of an internally truncated but functional DMD polypeptide, e.g., encoded by an exon 53-skipped DMD mRNA. In some embodiments, provided technologies provide one or more restored DMD functions, e.g., through truncated but functional DMD polypeptides. In some embodiments, provided technologies provide clinical benefits. In some embodiments, disease progression is delayed, slowed or prevented. In some embodiments, disease progression is delayed, slowed or prevented as assessed by a 10 meter walk test. In some embodiments, provided technologies reduce loss of pulmonary function relative to baseline. In some embodiments, muscle weakness in a subject is delayed, slowed or prevented. In some embodiments, muscle mass loss in a subject is delayed, slowed or prevented. In some embodiments, a subject improves in a muscular dystrophin assessment. In some embodiments, a subject improves in a DMD assessment. In some embodiments, improvement is achieved in one or more functional assessments, including North Star Ambulatory Assessment (NSAA) 2.0, Performance of the Upper Limb (PUL) 2.0, four-stair climb, handheld myometry, and pulmonary function tests. In some embodiments, such assessments are performed in such an order. In some embodiments, an assessment is or comprise lower limb motor function by timed function tests (including 10-meter walk/run time, four-stair climb time, and time to rise from the floor). In some embodiments, an assessment is or comprises upper limb proximal strength assessed by handheld myometer. In some embodiments, an assessment is or comprises pulmonary function tests (peak flow rate [PFR], cough peak flow [CPF], and FVC). In some embodiments, improvements are achieved independently in one or more or all these assessments. In some embodiments, an assessment is one disclosed in the Examples. In some embodiments, an improvement is compared to a baseline. In some embodiments, an improvement is compared to prior to WVE-N531 administration. In some embodiments, an improvement is compared to predicted level, severity and/or function with WVE-N531 administration. In some embodiments, an improvement is compared to absence of WVE-N531 administration. In some embodiments, an improvement is compared to administration of a reference composition. In some embodiments, a reference composition is comparable to an administered WVE-N531 composition but does not contain WVE-N531 or contains less amount of WVE-N531.

In some embodiments, provided technologies reduce loss of ambulation relative to baseline.

In some embodiments, provided technologies provide increases from baseline in dystrophin levels of about 1% or more of normal levels after administration for a certain time period or after a certain number of doses. In some embodiments, it is about 1% or more. In some embodiments, it is about 2% or more. In some embodiments, it is about 3% or more. In some embodiments, it is about 4% or more. In some embodiments, it is about 5% or more. In some embodiments, it is about 6% or more. In some embodiments, it is about 7% or more. In some embodiments, it is about 8% or more. In some embodiments, it is about 9% or more. In some embodiments, it is about 10% or more. In some embodiments, it is about 11% or more. In some embodiments, it is about 12% or more. In some embodiments, it is about 13% or more. In some embodiments, it is about 14% or more. In some embodiments, it is about 15% or more. In some embodiments, it is about 16% or more. In some embodiments, it is about 17% or more. In some embodiments, it is about 18% or more. In some embodiments, it is about 19% or more. In some embodiments, it is about 20% or more. In some embodiments, a time period is about 10-60 weeks. In some embodiments, it is about 4 weeks. In some embodiments, it is about 8 weeks. In some embodiments, it is about 10 weeks. In some embodiments, it is about 12 weeks. In some embodiments, it is about 13 weeks. In some embodiments, it is about 14 weeks. In some embodiments, it is about 15 weeks. In some embodiments, it is about 16 weeks. In some embodiments, it is about 20 weeks. In some embodiments, it is about 24 weeks. In some embodiments, it is about 25 weeks. In some embodiments, it is about 28 weeks. In some embodiments, it is about 30 weeks. In some embodiments, it is about 32 weeks. In some embodiments, it is about 35 weeks. In some embodiments, it is about 36 weeks. In some embodiments, it is about 37 weeks. In some embodiments, it is about 38 weeks. In some embodiments, it is about 40 weeks. In some embodiments, it is about 45 weeks. In some embodiments, it is about 48 weeks. In some embodiments, it is about 49 weeks. In some embodiments, it is about 50 weeks. In some embodiments, it is about 72 weeks. In some embodiments, it is about 73 weeks. In some embodiments, it is about 74 weeks. In some embodiments, it is about 96 weeks. In some embodiments, it is about 97 weeks. In some embodiments, it is about 98 weeks or more. In some embodiments, it is about 1, 2, 3, 4, 5, 6, 7, 8, 9, 10, 11, 12, 13, 14, 15, 16, 17, 18, 19, or 20 months. In some embodiments, a certain number of doses is about 5 doses. In some embodiments, it is about 6 doses. In some embodiments, it is about 7 doses. In some embodiments, it is about 8 doses. In some embodiments, it is about 9 doses. In some embodiments, it is about 10 doses. In some embodiments, it is about 11 doses. In some embodiments, it is about 12 doses. In some embodiments, it is about 13 doses. In some embodiments, it is about 14 doses. In some embodiments, it is about 15 doses. In some embodiments, it is about 16 doses. In some embodiments, it is about 17 doses. In some embodiments, it is about 18 doses. In some embodiments, it is about 19 doses. In some embodiments, it is about 20 doses. In some embodiments, it is about 21 doses. In some embodiments, it is about 22 doses. In some embodiments, it is about 23 doses. In some embodiments, it is about 24 doses. In some embodiments, it is about 25 doses. In some embodiments, it is about 30 doses. In some embodiments, it is about 35 doses. In some embodiments, it is about 40 doses. In some embodiments, it is about 45 doses. In some embodiments, it is about 48 doses. In some embodiments, it is about 49 doses. In some embodiments, it is about 50 or more doses. In some embodiments, WVE-N531 is administered every two weeks.

In some embodiments, provided technologies do not lead to adverse events or low levels of adverse events, or no or low levels of severe and/or serious adverse events. In some embodiments, adverse events associated with provided technologies are tolerable or manageable.

In some embodiments, the present disclosure provides technologies for manufacturing WVE-N531. In some embodiments, the present disclosure provides technologies for manufacturing a WVE-N531 drug substance. In some embodiments, the present disclosure provides technologies for manufacturing a WVE-N531 drug product. In some embodiments, the present disclosure provides technologies for characterizing WVE-N531 drug substance and/or product. In some embodiments, the present disclosure provides release specifications for characterizing WVE-N531 drug substance and/or product. In some embodiments, the present disclosure provides WVE-N531, compositions thereof, drug substances and/or products thereof manufactured by provided processes.

### BRIEF DESCRIPTION OF THE DRAWING

FIG. 1. An example flow diagram for WVE-N531 drug product manufacturing process.

FIG. 2. WVE-N531 provides potent exon 53 skipping in non-human primates. (A): an example regimen. Monkey animals received 6×weekly IV infusions of PBS or 3, 7, or 25 mg/kg WVE-N531 (n=2 per dose). W: week. Necropsied on day 38. (B): Exon 53 skipping assessed by RT-PCR. Healthy animals have normal levels of dystrophin, but exon skipping can be assessed by detection of skipped transcript.

FIG. 3. Example schematics of a study design. Abbreviations: DSMB=Data Safety Monitoring Board; N=number of patients.

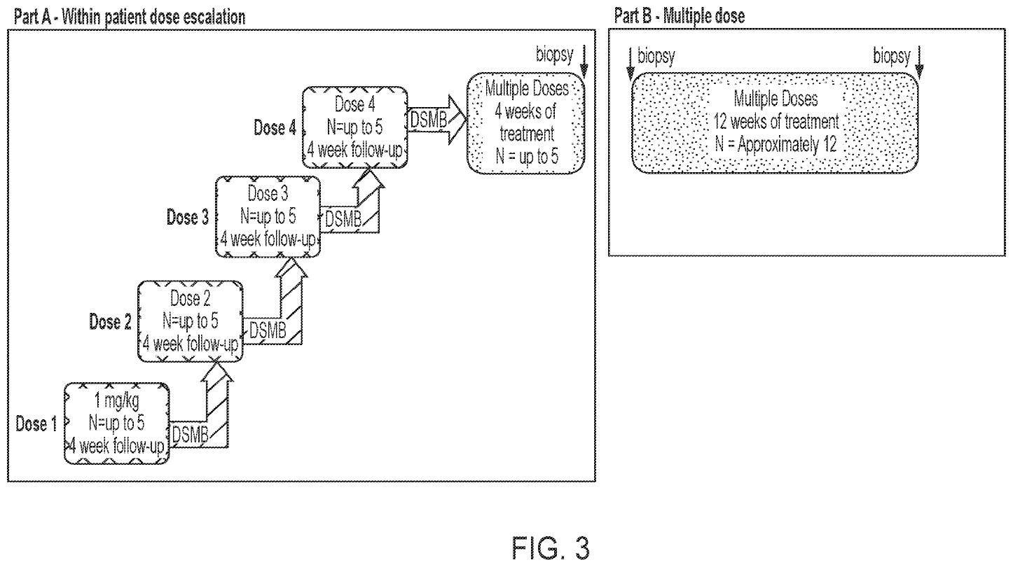

FIG. 4. WVE-N531 demonstrates increased plasma concentration in human subjects as compared to suvodirsen. Human subjects received an IV infusion of 1, 3, or 6 mg/kg WVE-N531 or 5 mg/kg suvodirsen. Following infusion, plasma concentration (μg/ml) of WVE-N531 or suvodirsen was measured across time for up to at least 42 days.

FIG. 5. WVE-N531 demonstrates increased plasma concentration in human subjects. Human subjects received an IV infusion of 1, 3, 6, or 10 mg/kg WVE-N531. Following infusion, plasma concentration (μg/ml) of WVE-N531 was measured across time for up to at least 105 days.

FIG. 6. WVE-N531 demonstrates increased plasma concentration in human subjects as compared to suvodirsen. Human subjects received an IV infusion of 1, 3, 6, or 10 mg/kg WVE-N531 or 5 mg/kg suvodirsen. Following infusion, plasma concentration (μg/ml) of WVE-N531 or suvodirsen was measured across time for up to at least 21 days.

FIG. 7. Schematic of an example study design.

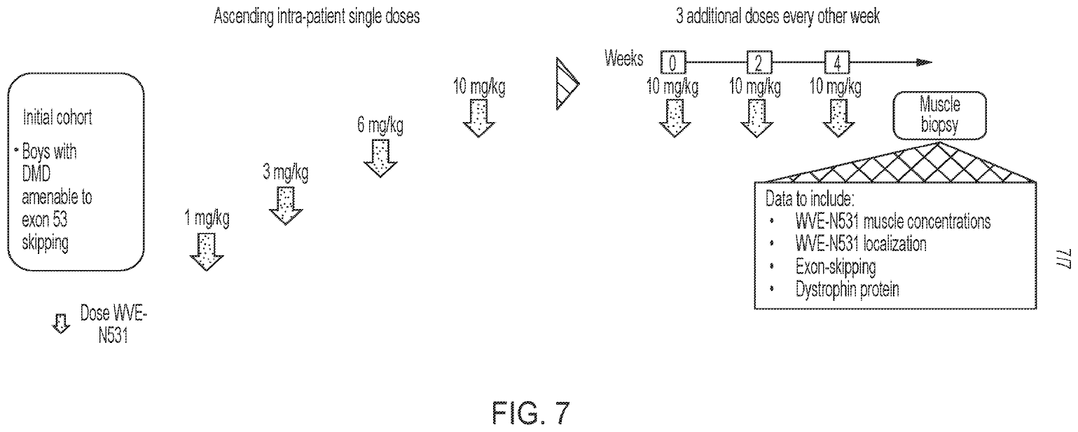

### DEFINITIONS

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; and (v) where ranges are provided, endpoints are included.

Unless otherwise specified, description of oligonucleotides and elements thereof (e.g., base sequence, sugar modifications, internucleotidic linkages, linkage phosphorus stereochemistry, etc.) is from 5′ to 3′. Unless otherwise specified, oligonucleotides described herein may be provided and/or utilized in various forms including salt forms, particularly pharmaceutically acceptable salt forms. As those skilled in the art will appreciate after reading the present disclosure, in some embodiments, oligonucleotides may be provided as salts such as sodium or potassium salts. As those skilled in the art will appreciate, in some embodiments, individual oligonucleotides within a composition may be considered to be of the same constitution and/or structure even though, within such composition (e.g., a liquid composition), particular such oligonucleotides might be in different form(s) including salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition)), and will understand that, so long as their acid forms (e.g., replacing all cations, if any, with H) are of the same constitution and/or structure, such individual oligonucleotides may properly be considered to be of the same constitution and/or structure.

Approximately: As used herein, the terms “about” and “approximately” may be understood to permit standard variation as would be understood by those of ordinary skill in the art. In some embodiments, the terms “approximately” or “about” in reference to a number are generally taken to include numbers that fall within a range of 5%, 10%, 15%, 20%, 25%, or 30%, in either direction (greater than or less than) of the number unless otherwise stated or otherwise evident from the context. In some embodiments, a range is +5%. In some embodiments, a range is +10%.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Pharmaceutical composition: As used herein, the term “pharmaceutical composition” refers to an active agent, formulated together with one or more pharmaceutically acceptable carriers. In some embodiments, active agent is present in a unit dose amount appropriate for administration in a therapeutic regimen that shows a statistically significant probability of achieving a controlled therapeutic effect when administered to a relevant population.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, a provided compound comprises more than one acid groups, for example, a provided oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), each acidic group having sufficient acidity independently exists as its salt form (e.g., in an oligonucleotide comprising natural phosphate linkages and phosphorothioate internucleotidic linkages, each of the natural phosphate linkages and phosphorothioate internucleotidic linkages independently exists as its salt form). In some embodiments, a pharmaceutically acceptable salt of an oligonucleotide is a sodium salt of a provided oligonucleotide. In some embodiments, a pharmaceutically acceptable salt of an oligonucleotide is a sodium salt of a provided oligonucleotide, wherein each acidic linkage, e.g., each natural phosphate linkage and phosphorothioate internucleotidic linkage, exists as a sodium salt form (all sodium salt).

Subject: As used herein, the term “subject” or “test subject” refers to any organism to which a provided compound or composition is administered in accordance with the present disclosure e.g., for experimental, diagnostic, prophylactic, and/or therapeutic purposes. In some embodiments, a subject may be suffering from, and/or susceptible to a disease, disorder, and/or condition. In some embodiments, a subject is a human subject.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: In some embodiments, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount. In some embodiments, a single dose is an infusion, which may take up to one or more hours.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Chirally controlled oligonucleotide composition: The terms “chirally controlled (stereocontrolled or stereodefined) oligonucleotide composition”, “chirally controlled (stereocontrolled or stereodefined) nucleic acid composition”, and the like, as used herein, refers to a composition that comprises a plurality of oligonucleotides (or nucleic acids, chirally controlled oligonucleotides or chirally controlled nucleic acids) which share 1) a common base sequence, 2) a common pattern of backbone linkages; 3) a common pattern of backbone chiral centers, and 4) a common pattern of backbone phosphorus modifications (oligonucleotides of a particular type), wherein the plurality of oligonucleotides (or nucleic acids) share the same stereochemistry at one or more chiral internucleotidic linkages (chirally controlled internucleotidic linkages, whose chiral linkage phosphorus is Rp or Sp, not a random Rp and Sp mixture as non-chirally controlled internucleotidic linkages). Level of the plurality of oligonucleotides (or nucleic acids) in a chirally controlled oligonucleotide composition is non-random (pre-determined, controlled). Chirally controlled oligonucleotide compositions are typically prepared through chirally controlled oligonucleotide preparation to stereoselectively form one or more chiral internucleotidic linkages (e.g., using chiral auxiliaries as exemplified in the present disclosure, compared to non-chirally controlled (stereorandom, non-stereoselective, racemic) oligonucleotide synthesis such as traditional phosphoramidite-based oligonucleotide synthesis using no chiral auxiliaries or chiral catalysts to purposefully control stereoselectivity). A chirally controlled oligonucleotide composition is enriched, relative to a substantially racemic preparation of oligonucleotides having the common base sequence, the common pattern of backbone linkages, and the common pattern of backbone phosphorus modifications, for oligonucleotides of the plurality. In some embodiments, a chirally controlled oligonucleotide composition comprises a plurality of oligonucleotides of a particular oligonucleotide type defined by: 1) base sequence; 2) pattern of backbone linkages; 3) pattern of backbone chiral centers; and 4) pattern of backbone phosphorus modifications, wherein it is enriched, relative to a substantially racemic preparation of oligonucleotides having the same base sequence, pattern of backbone linkages, and pattern of backbone phosphorus modifications, for oligonucleotides of the particular oligonucleotide type. As one having ordinary skill in the art readily appreciates, such enrichment can be characterized in that compared to a substantially racemic preparation, at each chirally controlled internucleotidic linkage, a higher level of the linkage phosphorus has the desired configuration. In some embodiments, each chirally controlled internucleotidic linkage independently has a diastereopurity of at least 80%, 85%, 90%, 91%, 92%, 93%, 94%, 95%, 96%, 97%, 98%, or 99% with respect to its chiral linkage phosphorus. In some embodiments, each independently has a diastereopurity of at least 90%. In some embodiments, each independently has a diastereopurity of at least 95%. In some embodiments, each independently has a diastereopurity of at least 97%. In some embodiments, each independently has a diastereopurity of at least 98%. In some embodiments, oligonucleotides of a plurality have the same constitution. In some embodiments, oligonucleotides of a plurality have the same constitution and stereochemistry, and are structurally identical.

In some embodiments, the plurality of oligonucleotides in a chirally controlled oligonucleotide composition share the same base sequence, the same, if any, nucleobase, sugar, and internucleotidic linkage modifications, and the same stereochemistry (Rp or Sp) independently at linkage phosphorus chiral centers of one or more chirally controlled internucleotidic linkages, though stereochemistry of certain linkage phosphorus chiral centers may differ. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%0, % 40% 50%, 60%0, % 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence are oligonucleotides of the plurality. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93% 94% 95% 96% 97%, 98%, or 99%, or at least 5%, 10%, 0% 30% 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications (e.g., of a plurality of oligonucleotide or an oligonucleotide type), or of all oligonucleotides in a composition that share a common base sequence, a common patter of base modifications, a common pattern of sugar modifications, a common pattern of internucleotidic linkage types, and/or a common pattern of internucleotidic linkage modifications (e.g., of a plurality of oligonucleotide or an oligonucleotide type), or of all oligonucleotides in a composition that share the same constitution, are oligonucleotides of the plurality. In some embodiments, a percentage is at least (DP)^{NCI}, wherein DP is a percentage selected from 85%-100%, and NCI is the number of chirally controlled internucleotidic linkage. In some embodiments, DP is at least 85%, 90%, 91%, 92%, 93%, 94%, 95%, 96%, 97%, 98%, or 99%. In some embodiments, DP is at least 85%. In some embodiments, DP is at least 90%. In some embodiments, DP is at least 95%. In some embodiments, DP is at least 96%. In some embodiments, DP is at least 97%. In some embodiments, DP is at least 98%. In some embodiments, DP is at least 99%. In some embodiments, DP reflects diastereopurity of linkage phosphorus chiral centers chirally controlled internucleotidic linkages. In some embodiments, diastereopurity of a linkage phosphorus chiral center of a internucleotidic linkage may be typically assessed using an appropriate dimer comprising such an internucleotidic linkage and the two nucleoside units being linked by the internucleotidic linkage. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 0.1%-100% (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. For instance, in some embodiments a chirally controlled oligonucleotide composition comprises one oligonucleotide type at a predetermined level (e.g., as described above). In some embodiments, a chirally controlled oligonucleotide composition comprises more than one oligonucleotide type, each independently at a predetermined level. In some embodiments, a chirally controlled oligonucleotide composition comprises multiple oligonucleotide types, each independently at a predetermined level. In some embodiments, a chirally controlled oligonucleotide composition is a composition of oligonucleotides of an oligonucleotide type, which composition comprises a predetermined level of a plurality of oligonucleotides of the oligonucleotide type.

### DETAILED DESCRIPTION OF CERTAIN EMBODIMENTS

Among other things, the present disclosure provides methods for treating muscular dystrophy, e.g., treatment of subjects having a mutation of the DMD gene that is amenable to exon 53 skipping, by administering an amount of WVE-N531 described herein to the subject. Dystrophin plays a vital role in muscle function, and various muscle-related diseases are characterized by mutated forms of this gene. In certain embodiments, methods described herein may be used for inducing exon skipping in transcripts, e.g., mRNA, of mutated forms of a human dystrophin gene, such as the mutated dystrophin gene forms found in muscular dystrophin, e.g., DMD and BMD. In some embodiments, provided technologies provide exon 53-skipped DMD mRNA. In some embodiments, present disclosure provide DMD polypeptides encoded by such DMD mRNA. In some embodiments, production of such DMD polypeptides restore, in some embodiments partially and in some embodiments fully, one or more DMD functions.

WVE-N531 can induce exon 53 skipping and dystrophin protein restoration. It is reported that mutations in the DMD gene that are amenable to exon 53 skipping occur in approximately 8% of subjects with DMD. As those skilled in the art appreciate, various DMD mutations amenable to exon 53 skipping have been reported, and various technologies are available to determine if a mutation is amenable to exon 53 skipping. In some embodiments, DMD mutations such as Δ3-52, Δ4-52, Δ5-52, Δ6-52, Δ9-52, Δ10-52, Δ11-52, Δ13-52, Δ14-52, Δ15-52, Δ16-52, Δ17-52, Δ19-52, Δ21-52, Δ23-52, Δ24-52, Δ25-52, Δ26-52, Δ27-52, Δ28-52, Δ29-52, Δ30-52, Δ31-52, Δ32-52, Δ33-52, Δ34-52, Δ35-52, Δ36-52, Δ37-52, Δ38-52, Δ39-52, Δ40-52, Δ41-52, Δ42-52, Δ43-52, Δ45-52, Δ47-52, Δ48-52, Δ49-52, Δ50-52, Δ51-52, Δ52, Δ54-58, Δ54-61, Δ54-63, Δ54-64, Δ54-66, Δ54-76, Δ54-77, etc. are amenable to exon 53 skipping. In some embodiments, a mutation is Δ45-52. In some embodiments, a mutation is Δ47-52. In some embodiments, a mutation is Δ48-52. In some embodiments, a mutation is Δ49-52. In some embodiments, a mutation is Δ50-52. In some embodiments, a mutation is Δ51-52. In some embodiments, a mutation comprises Δ52. In some embodiments, a mutation is Δ52. In some embodiments, WVE-N531 mediates effective exon 53 skipping of DMD to restore the reading frame so that a shorter but at least partially functional dystrophin can be produced—a resulting dystrophin protein is not necessarily the “wild-type” form of dystrophin, but is a truncated, yet functional form of dystrophin and provides increased level of one or more dystrophin functions compared to absence of WVE-N531/exon 53 skipping.

In some embodiments, WVE-N531 provides high muscle concentrations, e.g., at about or at least about 5 μg/g, 8 μg/g, 10 μg/g, 15 μg/g, 20 μg/g, 25 μg/g, 30 μg/g, 33 μg/g, 35 μg/g, 40 μg/g, 45 μg/g, 50 μg/g, 55 μg/g, 60 μg/g, 65 μg/g, 70 μg/g, 75 μg/g, 80 μg/g, 85 μg/g, 90 μg/g, etc., e.g., in various muscle. In some embodiments, such muscle concentrations are provided at a time point of about 6 weeks after an initial dose and/or after about 3 doses, wherein each dose is about 10 mg/kg WVE-N531. In some embodiments, WVE-N531 provides exon 53 skipping, e.g., at levels of about or at least about 10%, 15%, 20%, 25%, 30%, 35%, 40%, 45%, 50%, 55%, 60%, 65%, 70%, etc. In some embodiments, exon skipping is provided at a time point of about 6 weeks after an initial dose and/or after about 3 doses, wherein each dose is about 10 mg/kg WVE-N531 (which dose is significantly lower than golodirsen and/or viltolarsen).

In some embodiments, the present disclosure provides methods comprising using WVE-N531 to induce exon 53 skipping in DMD in muscle cells. In some embodiments, the present disclosure provides methods comprising using WVE-N531 to increase levels of functional dystrophin protein in muscle cells. It is useful in the prophylaxis and treatment of muscular dystrophy, especially those forms of muscular dystrophy that are amenable to exon 53 skipping. Among other things, methods described herein provide improved treatment options for subjects with muscular dystrophy and offer significant and practical advantages over alternate methods, e.g. methods using other oligonucleotide therapeutics.

In some embodiments, the present disclosure relates to improved methods for treating muscular dystrophy by inducing exon skipping in a subject. In some embodiments, exon 53 skipping in DMD in a subject induced by administering an effective amount of WVE-N531 as described herein. In some embodiments, the present disclosure relates to improved methods for treating muscular dystrophy, by restoring/increasing levels of dystrophin polypeptides and/or activities thereof (e.g., by restoring/increasing levels of functional dystrophin protein) in a subject.

In some embodiments, the present disclosure provides a method for treating DMD in a subject having a mutation in the DMD gene that is amenable to exon 53 skipping, comprising administering an effective dose amount of WVE-N531 (optionally as part of a composition, e.g., a pharmaceutical formulation or dosage form), e.g., equivalent to about 1, 2, 3, 4, 5, 6, 7, 8, 9, 10, 11, 12, 13, 14, 15, 16, 17, 18, 19, or 20 mg/kg WVE-N531 free acid form, at a suitable frequency (e.g., weekly, biweekly, every three weeks, every four weeks, every five weeks, every 6 weeks, every 7 weeks, every 8 weeks, every 9 weeks or every 10 weeks).

In some embodiments, the present disclosure provides a method for treating DMD in a subject suffering therefrom, wherein the subject has a mutation in the DMD gene that is amenable to exon 53 skipping, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

In some embodiments, the present disclosure provides a method for providing DMD exon skipping in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, the present disclosure provides a method for increasing level of exon 53-skipped DMD transcripts in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, the present disclosure provides a method for restoring DMD RNA reading frame in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, the present disclosure provides a method for increasing level of a truncated DMD polypeptide in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, the present disclosure provides a method for providing increased level of a DMD function in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form. In some embodiments, a truncated DMD polypeptide is encoded by an exon53-skipped DMD mRNA. In some embodiments, such an mRNA may lack one or more additional exons in addition to exon 53. In some embodiments, a subject has a mutation in the DMD gene that is amenable to exon 53 skipping. In some embodiments, a subject is suffering from or susceptible to a muscular dystrophy, e.g., DMD. In some embodiments, each dose of WVE-N531 is independently administered in a pharmaceutical composition independently comprising WVE-N531 and pharmaceutically acceptable carrier, wherein WVE-N531 in each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

WVE-N531 can be administered in various forms. For example, in many embodiments, WVE-N531 are administered as salt forms. In some embodiments, there is a single form in a composition. In some embodiments, there is a single form in a dose. In some embodiments, a form is a pharmaceutically acceptable salt form. In some embodiments, a form is WVE-N531 hexadecasodium salt. In some embodiments, there are two or more forms in a composition. In some embodiments, there are two or more forms in a dose. In some embodiments, each of the two or more forms is independently a pharmaceutically acceptable salt form. In some embodiments, one of them is WVE-N531 hexadecasodium salt. In some embodiments, a composition is a liquid composition comprising dissolved WVE-N531, e.g., in phosphate buffered saline. In some embodiments, there are one or more types of cations in a solution. In some embodiments, each is independently H⁺ or a metal cation. In some embodiments, a metal cation is Na⁺. In some embodiments, a metal cation is K⁺. In some embodiments, each metal cation is independently Na⁺ or K⁺.

In some embodiments, a dose is about 1-20 mg/kg. In some embodiments, a dose is about 1-5 mg/kg. In some embodiments, a dose is about 5-20 mg/kg. In some embodiments, a dose is about 5-10 mg/kg. In some embodiments, a dose is about 10-20 mg/kg. In some embodiments, a dose is about 10-15 mg/kg. In some embodiments, a dose is about 15-20 mg/kg. In some embodiments, a dose is about 1 mg/kg. In some embodiments, a dose is about 1 mg/kg. In some embodiments, a dose is about 2 mg/kg. In some embodiments, a dose is about 3 mg/kg. In some embodiments, a dose is about 4 mg/kg. In some embodiments, a dose is about 5 mg/kg. In some embodiments, a dose is about 6 mg/kg. In some embodiments, a dose is about 7 mg/kg. In some embodiments, a dose is about 8 mg/kg. In some embodiments, a dose is about 9 mg/kg. In some embodiments, a dose is about 10 mg/kg. In some embodiments, a dose is about 11 mg/kg. In some embodiments, a dose is about 12 mg/kg. In some embodiments, a dose is about 13 mg/kg. In some embodiments, a dose is about 14 mg/kg. In some embodiments, a dose is about 15 mg/kg. In some embodiments, a dose is about 16 mg/kg. In some embodiments, a dose is about 17 mg/kg. In some embodiments, a dose is about 18 mg/kg. In some embodiments, a dose is about 19 mg/kg. In some embodiments, a dose is about 20 mg/kg.

In some embodiments, a dose is administered as a pharmaceutical composition comprising WVE-N531 and a pharmaceutically acceptable carrier. In some embodiments, a pharmaceutical composition is a WVE-N531 solution as described herein, e.g., in a buffer. In some embodiments, a pharmaceutically acceptable carrier is isotonic. In some embodiments, it is a WVE-N531 solution in phosphate buffered saline as described herein.

In some embodiments, two or more (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 or more) doses are administered. In some embodiments, about 10 or more doses are administered. In some embodiments, about 20 or more doses are administered. In some embodiments, about 30 or more doses are administered. In some embodiments, about 40 or more doses are administered. In some embodiments, about 50 or more doses are administered. In some embodiments, about 60 or more doses are administered. In some embodiments, about 70 or more doses are administered. In some embodiments, about 80 or more doses are administered. In some embodiments, about 90 or more doses are administered. In some embodiments, about 100 or more doses are administered. In some embodiments, about 200 or more doses are administered. In some embodiments, about 500 or more doses are administered. In some embodiments, each does is independently about 1-20 mg/kg as described herein. In some embodiments, each dose is independently administered as a pharmaceutical composition comprising WVE-N531 and a pharmaceutically acceptable carrier as described herein. In some embodiments, two or more doses are about the same mg/kg. In some embodiments, each dose is about the same mg/kg. In some embodiments, pharmaceutical compositions administered for two or more doses are about the same in terms of components and their relative amounts. In some embodiments, pharmaceutical compositions administered for all doses are about the same in terms of components and their relative amounts. In some embodiments, one or more earlier doses are independently lower than one or more later doses. In some embodiments, one or more earlier doses independently are higher than one or more later doses.

In some embodiments, doses are administered about weekly, or about every 2, 3, 4, 5, 6, 7, 8, 9, or 10, or about every 1, 2, 3, 4, 5, 6, 7, 8, 9, or 10 months. In some embodiments, WVE-N531 are administered about every two weeks wherein each dose is independently as described herein. In some embodiments, WVE-N531 are administered about every four weeks wherein each dose is independently as described herein. In some embodiments, WVE-N531 are administered about every four or more weeks wherein each dose is independently as described herein. In some embodiments, each dose interval (time between a dose and its preceding or subsequent dose) is about the same. In some embodiments, one or more earlier dose intervals are independently longer than one or more later ones. In some embodiments, one or more earlier dose intervals are independently shorter than one or more later ones.

In some embodiments, “about” is +/−1% (99% to 101% of a value). In some embodiments, it is +/−2%. In some embodiments, it is +/−3%. In some embodiments, it is +/−4%. In some embodiments, it is +/−5%. In some embodiments, it is +/−6%. In some embodiments, it is +/−7%. In some embodiments, it is +/−8%. In some embodiments, it is +/−9%. In some embodiments, it is +/−10%.

Unless specified otherwise, amounts, concentrations, doses, etc., of WVE-N531 are of the free acid form.

Amounts, concentrations, doses, etc. are typically purity corrected. In some embodiments, purity is measured by IP-RP-UPLC as described herein based on % area.

Vyondys 53™ (golodirsen) is an oligonucleotide of the phosphorodiamidate morpholino oligomer (PMO) subclass intended to increase dystrophin production via exon-skipping. Golodirsen was approved in 2019 in the US for the treatment of patients who have a confirmed mutation of the DMD gene that is amenable to exon 53 skipping. Approval was based on a mean change from baseline in dystrophin levels of 0.92% (standard deviation [SD]1.01) of normal levels following 48 weeks of treatment at 30 mg/kg/week. Viltepso™ (viltolarsen), another PMO subclass exon skipping ASO, was approved in the US in 2020 for the treatment of DMD patients amenable to exon 53 skipping. The approval was also based on an observed increase in dystrophin production with a mean increase of 5.3% (SD 4.5) of normal levels measured following 25 weeks of treatment at 80 mg/kg/week. Both golodirsen and viltolarsen were approved via the US accelerated approval mechanism on the basis of changes in the surrogate endpoint of dystrophin production, and the clinical benefit of either drug has not yet been demonstrated in patients with DMD at approval. Continued approval in the US is contingent upon verification of a clinical benefit in confirmatory trials. At the time of the present invention neither drug is approved in the United Kingdom or European Union (EU).

Among other things, provided technologies provide various advantages compared to golodirsen and/or viltolarsen, e.g., lower single doses, lower total doses, less frequent administration, improved exon 53 skipping in DMD, higher level of functional DMD polypeptides, improved clinical results, etc.

### Dystrophin

In some embodiments, the dystrophin (DMD) gene or a product thereof, or a variant or portion thereof, may be referred to as DMD, BMD, CMD3B, DXS142, DXS164, DXS206, DXS230, DXS239, DXS268, DXS269, DXS270, DXS272, MRX85, or dystrophin; External IDs: OMIM: 300377 MGI: 94909; HomoloGene: 20856; GeneCards: DMD; In Human: Entrez: 1756; Ensembl: ENSG00000198947; UniProt: P11532; RefSeq (mRNA): NM_000109; NM 004006; NM_004007; NM_004009; NM_004010; RefSeq (protein): NP_000100: NP_003997; NP_004000; NP_004001; NP_004002; Location (UCSC): Chr X: 31.1-33.34 Mb; In Mouse: Entrez: 13405; Ensembl: ENSMUSG00000045103; UniProt: P11531; RefSeq (mRNA): NM_007868; NM_001314034; NM_001314035; NM_001314036; NM_001314037; RefSeq (protein): NP_001300963; NP_001300964; NP_001300965; NP_001300966; NP_001300967; Location (UCSC): Chr X: 82.95-85.21 Mb.

Dystrophin is reported to be found in muscle cells and is crucial in strengthening and protecting muscle fibers. Normal dystrophin protein is reported to be part of a protein complex called the dystrophin-glycoprotein complex, which according to various reports provides structural stability to skeletal muscle and protects the muscle from injury during contraction and relaxation. In addition, dystrophin is reported to be essential for cell survival via a transmembrane signaling function and modulation of vasomotor response to physical activity.

The DMD gene reportedly contains 79 exons distributed over 2.3 million bp of genetic real estate on the X chromosome; however, only approximately 14,000 bp (<1%) is reported to be used for translation into protein (coding sequence). It is reported that about 99.5% of the genetic sequence, the intronic sequences, is spliced out of the 2.3 million bp initial heteronuclear RNA transcript to provide a mature 14,000 bp mRNA that includes all key information for dystrophin protein production. In some embodiments, subjects with DMD have mutation(s) in the DMD gene that prevent the appropriate construction of the wild-type DMD mRNA and/or the production of the wild-type dystrophin protein, and subjects with DMD often show marked dystrophin deficiency in their muscle.

In some embodiments, a dystrophin transcript, e.g., mRNA, or protein encompasses those related to or produced from alternative splicing. For example, at least sixteen alternative transcripts of the dystrophin gene were reported following an analysis of splicing patterns of the DMD gene in skeletal muscle, brain and heart tissues.

It is reported that dystrophin has several isoforms. In some embodiments, dystrophin refers to a specific isoform. At least three full-length dystrophin isoforms have been reported, each controlled by a tissue-specific promoter. The muscle isoform is reportedly mainly expressed in skeletal muscle but also in smooth and cardiac muscles, the brain dystrophin is reportedly specific for cortical neurons but can also be detected in heart and cerebellar neurons, while the Purkinje-cell type reportedly accounts for nearly all cerebellar dystrophin. Alternative splicing reportedly provides a means for dystrophin diversification: the 3′ region of the gene reportedly undergoes alternative splicing resulting in tissue-specific transcripts in brain neurons, cardiac Purkinje fibers, and smooth muscle cells while 12 patterns of alternative splicing have been reported in the 5′ region of the gene in skeletal muscle.

In some embodiments, a dystrophin mRNA, gene or protein is a revertant version.

Various mutations in the DMD gene can and/or were reported to cause muscular dystrophy. Mutations in the form of large deletions (1 or more exons) are reported to account for approximately two-thirds of all DMD gene mutations; the remaining mutations are reported to be due to duplications and small deletions, insertions, point mutations, or splicing mutations. According to certain reports, deletions are typically clustered in a hotspot region between exons 45 and 55 and prevent translation of dystrophin. Absent or defective dystrophin protein, resulting from DMD gene mutations, is reported to disrupt the dystrophin-glycoprotein complex, leading to increased muscle membrane fragility, chronic muscle damage, inflammation, replacement of muscle fibers with fat and fibrotic tissue, and then loss of muscle function.

### Muscular Dystrophy

Muscular dystrophy (MD) is any of a group of muscle conditions, diseases, or disorders that results in (increasing) weakening and breakdown of skeletal muscles over time. The conditions, diseases, or disorders differ in which muscles are primarily affected, the degree of weakness, when symptoms begin, and how quickly symptoms worsen. Many MD subjects eventually become unable to walk. In many cases muscular dystrophy is fatal. Some types are also associated with problems in other organs, including the central nervous system. In some embodiments, a muscular dystrophy is Duchenne (Duchenne's) Muscular Dystrophy (DMD). In some embodiments, a muscular dystrophy Becker (Becker's) Muscular Dystrophy (BMD).

In some embodiments, a symptom of Duchenne Muscular Dystrophy is muscle weakness associated with muscle wasting, with the voluntary muscles being first affected, especially those of the hips, pelvic area, thighs, shoulders, and calves. Muscle weakness can also occur later, in the arms, neck, and other areas. Calves are often enlarged. Symptoms usually appear before age six and may appear in early infancy. Other physical symptoms are: awkward manner of walking, stepping, or running (in some cases, subjects tend to walk on their forefeet, because of an increased calf muscle tone), frequent falls, fatigue, difficulty with motor skills (e.g., running, hopping, jumping), lumbar hyperlordosis, possibly leading to shortening of the hip-flexor muscles, unusual overall posture and/or manner of walking, stepping, or running, muscle contractures of Achilles tendon and hamstrings impair functionality, progressive difficulty walking, muscle fiber deformities, pseudohypertrophy (enlarging) of tongue and calf muscles, higher risk of neurobehavioral disorders (e.g., ADHD), learning disorders (e.g., dyslexia), and non-progressive weaknesses in specific cognitive skills (e.g., short-term verbal memory), which are believed to be the result of absent or dysfunctional dystrophin in the brain, eventual loss of ability to walk (usually by the age of 12), skeletal deformities (including scoliosis in some cases), and trouble getting up from lying or sitting position. In some embodiments, provided technologies delay onset of, slow progression of, reduce severity of, and/or prevent one or more symptoms.

In some embodiments, Becker muscular dystrophy (BMD) is caused by mutations that give rise to shortened but in-frame transcripts resulting in the production of truncated but partially functional protein(s). Such partially functional protein(s) were reported to retain the critical amino terminal, cysteine rich and C-terminal domains but usually lack elements of the central rod domains which were reported to be of less functional significance. England et al. 1990 Nature, 343, 180-182.

In some embodiments, BMD phenotypes range from mild DMD to virtually asymptomatic, depending on the precise mutation and the level of dystrophin produced. Yin et al. 2008 Hum. Mol. Genet. 17: 3909-3918.

In some embodiments, dystrophy subjects with out-of-frame mutations are generally diagnosed with the more severe Duchenne Muscular Dystrophy, and dystrophy subjects with in-frame mutations are generally diagnosed with the less severe Becker Muscular Dystrophy.

### Exon Skipping as a Treatment for Muscular Dystrophy

Exon skipping can induce cellular machinery to skip over one or more targeted exons and restore the reading frame resulting in the production of internally truncated but functional dystrophin polypeptides. In various exon-skipping approaches, oligonucleotides can bind to target complementary sequences in the messenger ribonucleic acids (mRNAs), which engage with the splicing machineries to exclude exons from final transcripts. In subjects with DMD, this approach may slow disease progression by converting severe DMD symptoms to the milder symptoms such as those seen in patients with Becker muscular dystrophy (BMD), in which patients are ambulatory longer and have a longer life expectancy.

Various DMD genotypes are amenable to exon skipping including exon 53 skipping. Exon skipping can restore DMD mRNA reading frame, resulting in the production of a shorter but functional dystrophin protein. Exon 53 skipping is applicable, but not limited to, patients with deletions in the DMD gene of exons 43-52, 45-52, 47-52, 48-52, 49-52, 50-52, 51-52, or exon 52 alone. According to some reports, approximately 8% to 10% of patients with DMD may be treated by exon 53 skipping. Thirty-six month longitudinal natural history data in a cohort of patients with deletions amenable to skip exons 44, 45, 51, or 53 showed that patients amenable to exon 53 skipping in particular appear to have earlier onset of decline and are at a higher risk of dramatic functional loss (e.g., loss of ambulation) compared to other subgroups.

In some embodiments, exon skipping restores or maintains a proper reading frame, and/or creating an artificially internally truncated DMD protein which provides at least partially improved or fully restored biological activity. In some embodiments, an internally truncated DMD protein produced from a dystrophin transcript with a skipped exon(s) is more functional than a terminally truncated DMD protein e.g., produced from a dystrophin transcript with an out-of-frame deletion.

In some embodiments, an internally truncated DMD protein produced from a dystrophin transcript with a skipped exon(s) is more resistant to nonsense-mediated decay, which can degrade a terminally truncated DMD protein, e.g., produced from a dystrophin transcript with an out-of-frame deletion.

In some embodiments, a DMD subject is analyzed for DMD genotype and is confirmed to have a mutation of the DMD gene that is amenable to exon 53 skipping prior to administration of WVE-N531. In some embodiments, a mutation of the DMD gene that is amenable to exon 53 skipping is or comprises one or more nucleobase changes (e.g., replaced by a different nucleobase), insertions, and/or deletions, etc., wherein exon 53 skipping can produce a DMD polypeptide that can provided improved functions compared to without exon 53 skipping.

Prior to the present disclosure, Emflaza® (deflazacort) is a corticosteroid approved in the US for the treatment of DMD. Emflaza was shown to improve muscle strength (mean change 0.15 points as measured by the modified Medical Research Council sale) following a 12-week treatment period at a dose of 0.9 mg/kg/day. In a long-term placebo-controlled study in children with DMD aged 6 to 12 years, there was no significant effect of deflazacort on average muscle strength score at 2 years. While Emflaza is not approved for the treatment of DMD in the European Union (EU), corticosteroids such as deflazacort are commonly used off-label in the EU in patients with DMD to slow the decline in muscle strength, as well as to prolong ambulation and respiratory function However, the chronic use of corticosteroids has been associated with serious side effects such as immunosuppression and increased risk of infection, changes in endocrine function, hypertension, cataracts, bone demineralization, gastrointestinal perforation, and growth retardation in children. Further, corticosteroids do not correct the underlying genetic defect in DMD.

Vyondys 53™ (golodirsen) is an oligonucleotide of the phosphorodiamidate morpholino oligomer (PMO) subclass intended to increase dystrophin production via exon 53 skipping. Golodirsen was approved in 2019 in the US for the treatment of patients who have a confirmed mutation of the DMD gene that is amenable to exon 53 skipping. Approval was based on a mean change from baseline in dystrophin levels of 0.92% (standard deviation [SD] 1.01) of normal levels following 48 weeks of treatment at 30 mg/kg/week. Viltepso™ (viltolarsen), another PMO subclass exon skipping oligonucleotide, was approved in the US and Japan in 2020 for the treatment of patients with DMD who have a mutation amenable to exon 53 skipping. The approval was also based on an observed increase in dystrophin production with a mean increase of 5.3% (SD 4.5) of normal levels measured following 25 weeks of treatment at 80 mg/kg/week. Both golodirsen and viltolarsen were approved via the US accelerated approval mechanism on the basis of an increase in the surrogate endpoint of dystrophin production and neither drug has been shown to provide clinical benefit in patients with DMD. Continued approval in the US is contingent upon verification of a clinical benefit in confirmatory trials. Neither drug is approved in the United Kingdom (UK) or EU prior to the present disclosure.

Similarly, some patients may have a mutation that makes them eligible for treatment with both an exon 53-skipping oligonucleotide and Translarna™ (ataluren) or Exondys 51® (eteplirsen). Ataluren is a small molecule intended to enhance ribosomal read-through of nonsense mutations that is not approved for use in the United States (US). It received conditional marketing approval in the EU for the treatment of DMD resulting from a nonsense mutation in the dystrophin gene in ambulatory patients aged 5 years or older. Clinical efficacy of ataluren has not been established in non-ambulatory patients and additional evidence is required in order to support its continued approval in the EU. It is anticipated that ataluren's use would be limited to a small percentage of the DMD population in consideration of the low prevalence of nonsense mutations and its limited indication for ambulatory patients. Eteplirsen is an oligonucleotide of the PMO subclass intended to increase dystrophin production via exon skipping. Eteplirsen was approved in the US for the treatment of patients who have a confirmed mutation of the DMD gene that is amenable to exon 51 skipping. Approval was based on detection of an average dystrophin protein level of 0.93% of the dystrophin level in normal muscle as well as a median increase in truncated dystrophin expression of 0.1% in the only study where an estimate of dystrophin expression in response to eteplirsen treatment was possible. The drug is not approved in the EU prior to the present disclosure.

The plasma Cₘₐₓ and area under the plasma concentration-time curve (AUC) of golodirsen increases proportionally with dose and half-life is approximately 3 hours with no plasma accumulation observed. Based on a Phase 1 study of viltolarsen, the urinary excretion rate indicated removal of 70% to 80% of the total administered drug within 24 hours, indicating a short half-life (<24 hours). The kidney function of all patients was in the normal range. Eteplirsen is rapidly cleared from the plasma with the majority of drug elimination occurring within 24 hours. Twenty-four hours after the end of the infusion, mean concentrations of eteplirsen were 0.07% of the maximum (peak) concentration of drug in plasma (Cₘₐₓ). Renal clearance of eteplirsen accounts for over 60% of the administered dose with a terminal half-life (t_{1/2}) of 3 to 4 hours. Accumulation of eteplirsen during once-weekly dosing has not been observed.

Among other things, the present disclosure provides technologies for treating DMD, inducing DMD exon 53 skipping, increasing levels of exon 53-skipped DMD transcripts, producing or increasing levels of truncated DMD polypeptides from exon 53 skipping, increasing levels of and/or restoring one or more DMD functions, etc., comprising administering to a subject WVE-N531 or a composition thereof in accordance with the present disclosure, wherein the subject has a mutated DMD amenable to exon 53 skipping. In some embodiments, provided technologies provide various advantages over one or more existing approved drugs, for example, improved safety, tolerability, clearance, accumulation, exon skipping, clinical effects, and/or one or more other pharmacokinetic or pharmacodynamic aspects.

### WVE-N531

WVE-N531 may be described as (as those skilled in the art appreciate. 5′ to 3′ unless specified otherwise):
fC*SfU*SfCn001RfC*SfG*SfGn001RfUJ*SfU*SmCfU*SmG*SfA*SmAfG*SfG*SfU*SfGn001RffU*SfU*SfC
m: 2′-OMe modified nucleoside;
f: 2′-F modified nucleoside;
*S: Sp phosphorothioate linkage; and
n001R: Rp N-(1,3-dimethylimidazolidin-2-ylidenyl) phosphoramidate linkage.

WVE-N531 may be named by accepted oligonucleotide nomenclature: (Sp)-2′-fluoro-2 deoxy-P-thio-cytidylyl-(O^{3′}→O^{5′})(Sp)-2′-fluoro-2 deoxy-P-thio-uridylyl-(O^{3′}→O^{5′})-(Rp)-2 fluoro-2′-deoxy-P-((1,3-dimethylimidazolidin-2-ylidenyl)amino)-cytidylyl-(O^{3′}→O^{5′})-(Sp)-2′-fluoro-2′-deoxy-P-thio-cytidylyl-(O^{3′}→O^{5′})-(Sp)-2′-fluoro-2′-deoxy-P-thio-guanylyl-(O^{3′}→O^{5′})-(Rp)-2′-fluoro-2′-deoxy-P-((1,3-dimethylimidazolidin-2-ylidenyl)amino-guanylyl(O^{3′}→O^{5′})-(Sp)-2-fluoro-2′-deoxy-P-thio-uridylyl-(O^{3′}→O^{5′})-(Sp)-2′-fluoro-2′-deoxy-P-thio-uridylyl-(O^{3′}→O^{5′})-2′-O-methylcytidylyl-(O^{3′}→O^{5′})-(Sp)-2′-fluoro-2′-deoxy-P-thio-uridylyl-(O^{3′}→O^{5′})-(Sp)-2′-O-methyl-P-thio-guanylyl-(O^{3′}→O^{5′})-(Sp)-2′-fluoro-2′-deoxy-P-thio-adenylyl-(O^{3′}→O^{5′})-2′-O-methyladenylyl-(O^{3′}→O^{5′})-(Sp)-2′-fluoro-2-deoxy-P-thio-guanylyl-(O^{3′}→O^{5′})-(Sp)-2′-fluoro-2′-deoxy-thio-guanylyl-(O^{3′}→O^{5′})-(Sp)-2′-fluoro-2′-deoxy-P-thio-uridylyl(O^{3′}→O^{5′})-(Rp)-2′-fluoro-2′-deoxy-P-((1,3-dimethylimidazolidin-2-ylidenyl)amino)-guanylyl-(O^{3′}→O^{5′})-(Sp)-2′-fluoro-2′-deoxy-P-thio-uridylyl-(O^{3′}→O^{5′})-(Sp)-2′-fluoro-2′-deoxy-P-thio-uridylyl (O^{3′}→O^{5′})-2′-fluoro-2′-deoxycytidine.

In some embodiments, WVE-N531 is provided in a composition, e.g., a pharmaceutical composition, and/or administered as one or more pharmaceutically acceptable salts. In some embodiments, the present disclosure provides pharmaceutically acceptable salts of WVE-N531. In some embodiments, a pharmaceutically acceptable salt is a sodium salt. In some embodiments, the present disclosure provides WVE-N531 hexadecasodium salt. In some embodiments, a WVE-N531 drug substance is WVE-N531 hexadecasodium salt.

Molecular formulae and molecular weights of WVE-N531 and its hexadecasodium salt is described below:
Molecular Formula (free acid form): C₂₀₆H₂₅₀F₁₇N₇₈O₁₀₉P₁₉S₁₄
Molecular Weight (free acid form): 6923.02 g/mol
Molecular Formula (hexadecasodium salt form): C₂₀₆H₂₃₄F₁₇N₇₈O₁₀₉P₁₉S₁₄Na₁₆
Molecular Weight (hexadecasodium salt form): 7274.73 g/mol

WVE-N531 has 17 stereodefined internucleotide linkages, 14 of which are identified as Sp phosphorothioate linkages, and three as Rp N-(1,3-dimethylimidazolidin-2-ylidenyl) phosphoramidate linkages. Its internucleotide linkages can be illustrated as: 5′-SSRSSRSSOSSSOSSSRSS-3′, where ‘S’, ‘R’, and ‘O’ represent Sp phosphorothioate linkage, Rp N-(1,3-dimethylimidazolidin-2-ylidenyl) phosphoramidate linkage, and phosphate linkages, respectively. A N-(1,3-dimethylimidazolidin-2-ylidenyl) phosphoramidate linkage may be referred to as a n001 linkage.

The structure of WVE-N531 may be depicted as Formula I:

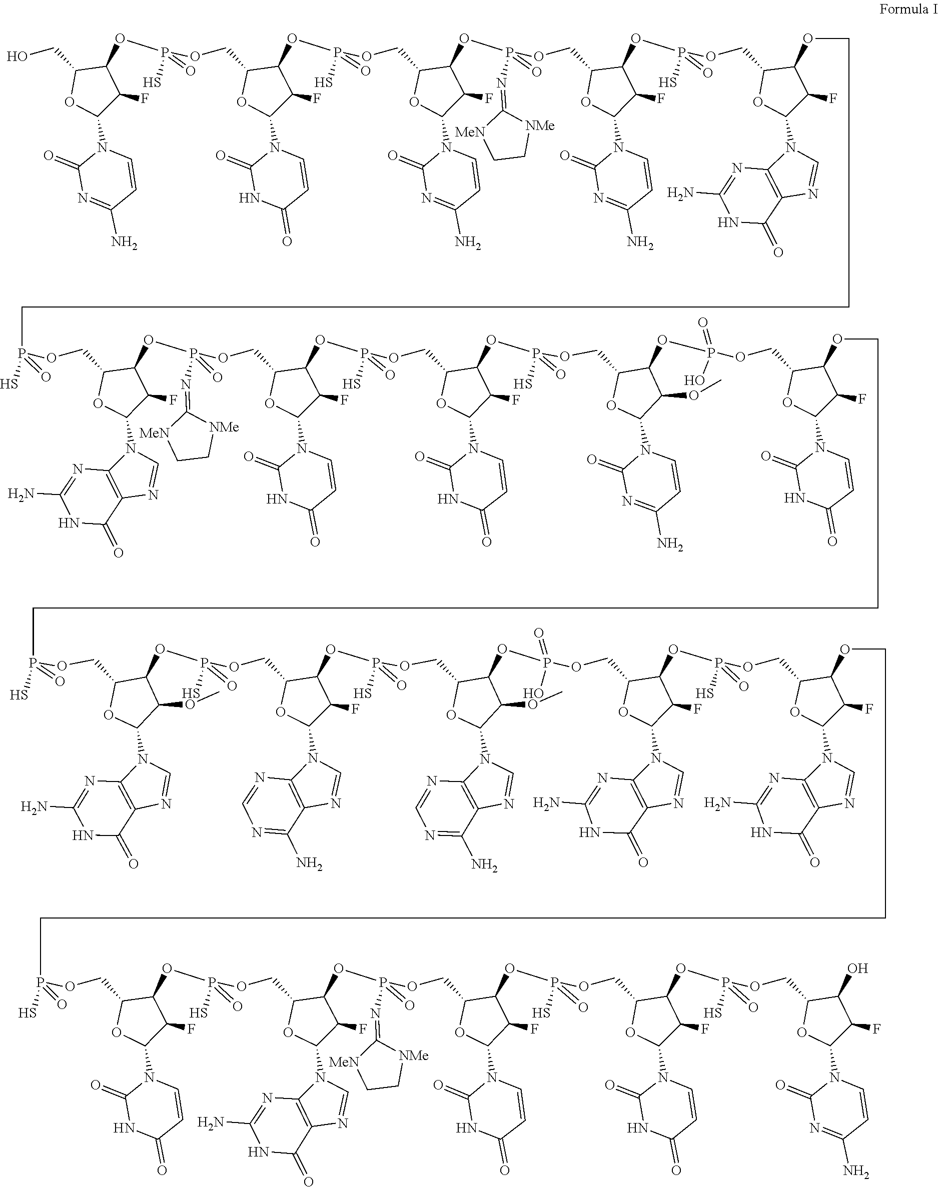

WVE-N531 hexadecasodium salt may be described as Formula I-a:

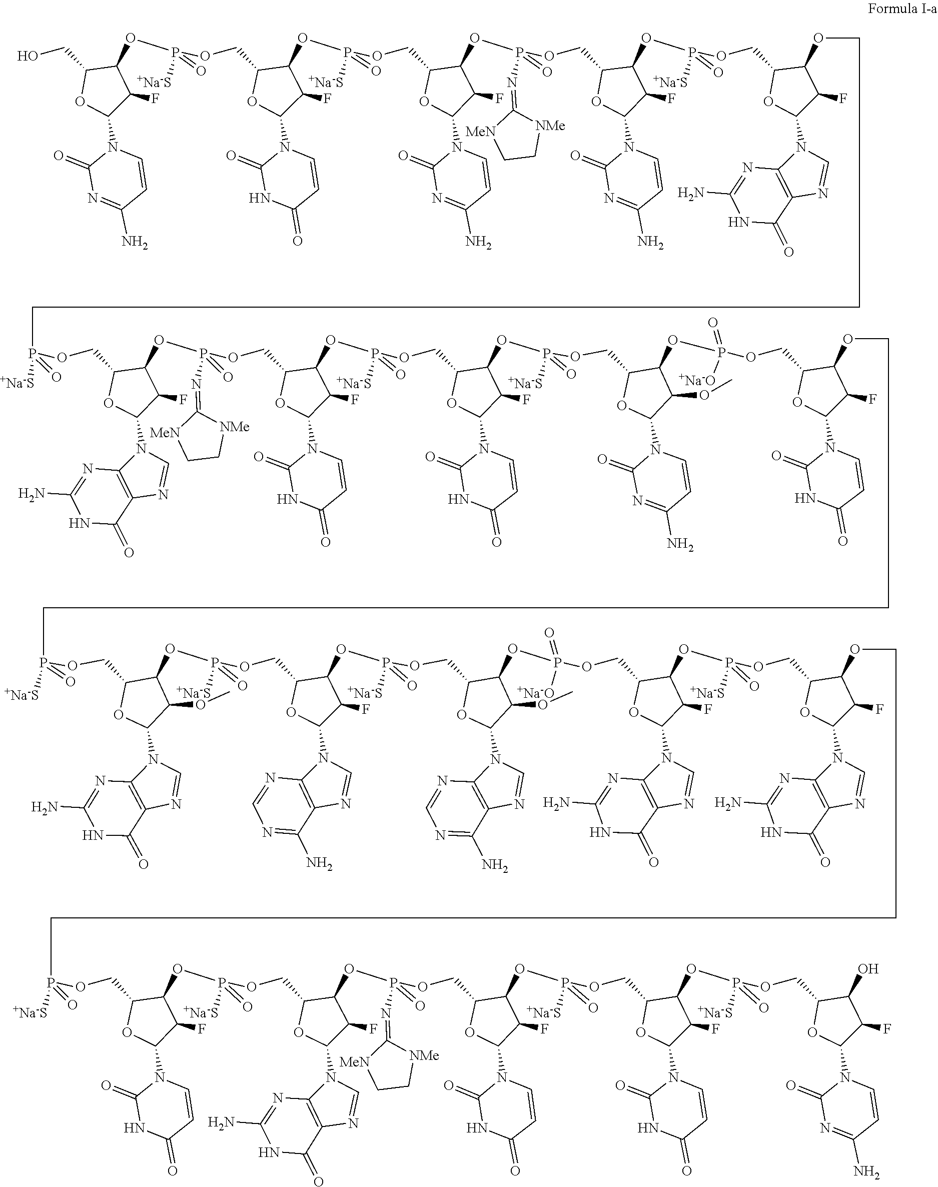

In Formulae I and I-a, due to the sizes of the structures, the structures are presented as 4 rows, in each of which there are five nucleoside units. As shown in there Formulae, each linkage in WVE-N531 is independently a Sp phosphorothioate linkage, a Rp n001 linkage or a natural phosphate linkage, and in a WVE-N531 salt, acidic hydrogen atoms of the phosphorothioate and phosphate linkages may be independently replaced with cations, e.g., in the hexadecasodium salt, each replaced with sodium.

Water solution of WVE-N531 hexadecasodium salt is typically a clear, colorless to pale yellow solution, and can have a concentration of about 150 mg/mL or more as determined by UV. In some embodiments, pH of WVE-N531 hexadecasodium salt in purified water ranges from 6.0-8.0.

Extinction coefficient of WVE-N531 is 186,486 M⁻¹ cm⁻¹ (determined in water) at 260 nm. Unless noted otherwise, when UV is utilized to measure concentration/amount of WVE-N531, 260 nm is utilized and this number is utilized in calculation. Using a molecular weight of 6918.72 g/mol (free acid form), this equates to an absorptivity factor of 27.0 OD/mg.

In some embodiments, WVE-N531 is supplied as an isotonic solution for dilution for IV infusion. In some embodiments, the total volume of infusion is 100-500 mL based on body weight of a subject. In some embodiments, infusion is administered over approximately a period of 1 hour. In some embodiments, infusion is extended to about 3 hours, e.g., to enhance tolerability. In some embodiments, WVE-N531 diluted infusion solution contains no preservatives and is administered without delay or within four hours of dilution. In some embodiments, 0.45% sodium chloride injection is utilized for dilution. In some embodiments, 0.9% sodium chloride injection is utilized for dilution.

In some embodiments, WVE-N531 is provided and administered as chirally controlled composition in which WVE-N531 is enriched over other diastereomers.

Among other things, the present disclosure provides WVE-N531 of high purity. In some embodiments, WVE-N531 has a level of purity of about 70%-90%, e.g., about 70%-85%, about 70%-80%, about 75%-85%, or about 70%, 71%, 72%, 3% 74%, 75%, 76%, 77% 78%, 79%, 80%, 81%, 82%, 83%, 84%, 85%, 86%, 87%, 88%, 89% or 90%. In some embodiments, a level of purity is determined by IP-RP-UPLC described herein. In some embodiments, it is about 70% as determined by IP-RP-UPLC described herein. In some embodiments, it is about 71% as determined by IP-RP-UPLC described herein. In some embodiments, it is about 72% as determined by IP-RP-UPLC described herein. In some embodiments, it is about 73% as determined by IP-RP-UPLC described herein. In some embodiments, it is about 74% as determined by IP-RP-UPLC described herein. In some embodiments, it is about 75% as determined by IP-RP-UPLC described herein. In some embodiments, it is about 76% as determined by IP-RP-UPLC described herein. In some embodiments, it is about 77% as determined by IP-RP-UPLC described herein. In some embodiments, it is about 78% as determined by IP-RP-UPLC described herein. In some embodiments, it is about 79% as determined by IP-RP-UPLC described herein. In some embodiments, it is about 80% as determined by IP-RP-UPLC described herein. In some embodiments, it is about 81% or more as determined by IP-RP-UPLC described herein. In some embodiments, it is about 82% as determined by IP-RP-UPLC described herein. In some embodiments, it is about 83% as determined by IP-RP-UPLC described herein. In some embodiments, it is about 84% as determined by IP-RP-UPLC described herein. In some embodiments, it is about 85% as determined by IP-RP-UPLC described herein. In some embodiments, it is about 86% as determined by IP-RP-UPLC described herein. In some embodiments, it is about 87% as determined by IP-RP-UPLC described herein. In some embodiments, it is about 88% as determined by IP-RP-UPLC described herein. In some embodiments, it is about 89% as determined by IP-RP-UPLC described herein. In some embodiments, it is about 90% or more as determined by IP-RP-UPLC described herein. In some embodiments, an IP-RP-UPLC utilize the Set A parameters. In some embodiments, purity is % area at a given wavelength, e.g., around 260 nm.

In some embodiments, level of impurities is about 10%-30% or lower. In some embodiments, it is about 10%-30%. In some embodiments, it is about 15%-30%. In some embodiments, it is about 20%-30%. In some embodiments, In some embodiments, it is about 30% or lower. In some embodiments, it is about 29% or lower. In some embodiments, it is about 28% or lower. In some embodiments, it is about 27% or lower. In some embodiments, it is about 26% or lower. In some embodiments, it is about 25% or lower. In some embodiments, it is about 24% or lower. In some embodiments, it is about 23% or lower. In some embodiments, it is about 22% or lower. In some embodiments, it is about 21% or lower. In some embodiments, it is about 20% or lower. In some embodiments, it is about 19% or lower. In some embodiments, it is about 18% or lower. In some embodiments, it is about 17% or lower. In some embodiments, it is about 16% or lower. In some embodiments, it is about 15% or lower. In some embodiments, it is about 14% or lower. In some embodiments, it is about 13% or lower. In some embodiments, it is about 12% or lower. In some embodiments, it is about 11% or lower. In some embodiments, it is about 10% or lower. In some embodiments, level of impurity is assessed using IP-RP-UPLC. In some embodiments, an IP-RP-UPLC utilize the Set A parameters. In some embodiments, purity is % area at a given wavelength, e.g., around 260 nm.

In some embodiments, the present disclosure provides various technologies for characterizing WVE-N531 preparations. In some embodiments, the present disclosure provides technologies for assessing purity of WVE-N531 or compositions thereof.

In some embodiments, the present disclosure provides technologies for manufacturing WVE-N531 or compositions thereof. In some embodiments, the present disclosure provides technologies for releasing WVE-N531 preparations.

### Manufacturing

In some embodiments, the present disclosure provides technologies for manufacturing WVE-N531 or a sat thereof, e.g., WVE-N531 hexadecasodium salt. In some embodiments, the present disclosure provides technologies for manufacturing a WVE-N531 drug substance. In some embodiments, the present disclosure provides technologies for manufacturing a pharmaceutical composition comprising WVE-N531 or a pharmaceutically acceptable salt form thereof and a pharmaceutically acceptable carrier. In some embodiments, the present disclosure provides WVE-N531 drug products.

In some embodiments, WVE-N531 or a salt thereof, e.g., WVE-N531 hexadecasodium salt, is chemically manufactured in compliance with Good Manufacturing Practice (cGMP) regulations.

In some embodiments, a provided technology comprises a multi-step process that includes solid-phase oligonucleotide synthesis, cleavage of the crude protected oligonucleotide from the solid support, removal of protecting groups (deprotection), preparative anion exchange (AEX) chromatographic purification, concentration and/or desalting.

In some embodiments, stereochemistry of WVE-N531 or salts thereof is established through control of starting materials for synthesis as well as synthetic processes. For example, in some embodiments, provided technologies comprise use of phosphoramidites prepared from the chiral auxiliaries (L)-DPSE and (L)-PSM during coupling steps to ensure that the intended stereodefined Sp phosphorothioate and Rp n001 linkages respectively, are obtained.

A useful manufacturing process for WVE-N531 hexadecasodium salt as a drug substance and certain results are described below as an example.

### A Useful Manufacturing Process

In some embodiments, a manufacturing process consists of synthesizing the oligonucleotide, containing a combination of three kinds of internucleotide linkage (phosphodiester, stereodefined phosphorothioate, and stereodefined n001), on solid support using phosphoramidite chemistry, as shown in the synthesis cycles below. Each cycle to introduce a stereodefined phosphorothioate or stereodefined n001 consists of 5′-detritylation, coupling, capping of exposed chiral auxiliary secondary amine (Capping-1), thiolation or imidation respectively, and capping of unreacted 5′-hydroxyl groups (Capping-2). Each cycle to introduce a phosphodiester consists of detritylation, coupling, oxidation, and Capping-2. Each coupling reaction is carried out by activation of an appropriate phosphoramidite and reaction with the free 5′-hydroxyl group of a support-immobilized protected nucleotide or oligonucleotide. After the appropriate number of cycles and final detritylation, the (L)-PSM chiral auxiliary and cyanoethyl phosphate protecting groups are removed from a crude oligonucleotide by on-column treatment with diethylamine (DEA) in acetonitrile (ACN). The (L)-DPSE chiral auxiliary is then removed by treatment with NEt₃·HF (triethylamine hydrogen fluoride) solution. The crude oligonucleotide is then cleaved from the solid support and removed from the column by ammonia treatment. The base protecting groups are removed with continued ammonia treatment in an appropriately sized reaction vessel. The resulting crude oligonucleotide is purified using preparative anion exchange (AEX) high-performance liquid chromatography (HPLC) followed by concentration and desalting to yield WVE-N531 or a salt thereof, e.g., WVE-N531 hexadecasodium salt, in some embodiments, as drug substance.

### A Useful Cycle for Phosphorothioate Assembly

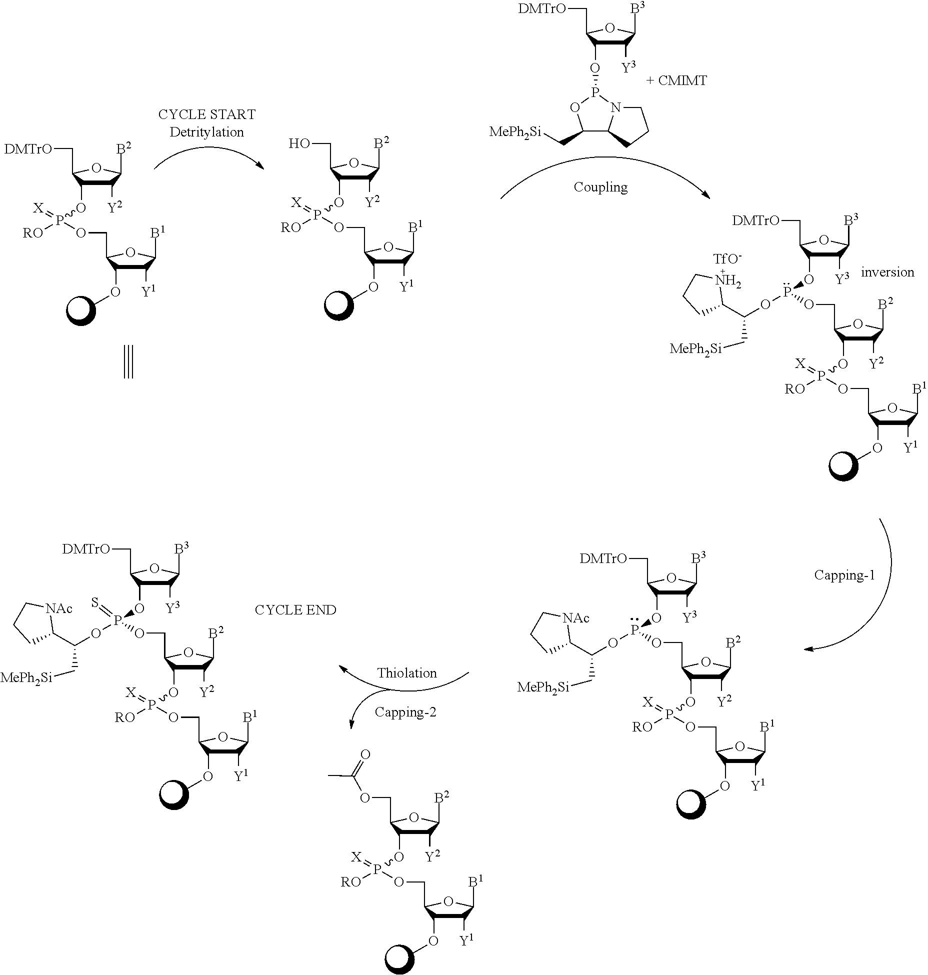

A Useful Cycle for n001 Assembly

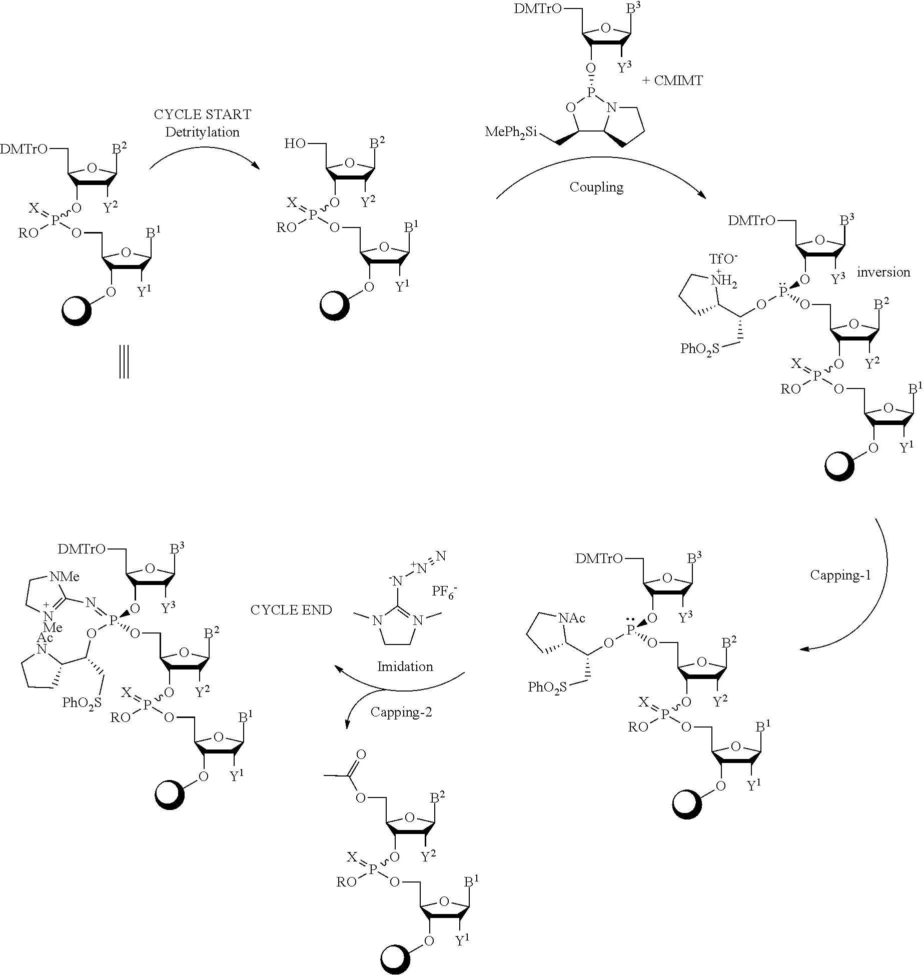

### A Useful Cycle for Phosphate Assembly

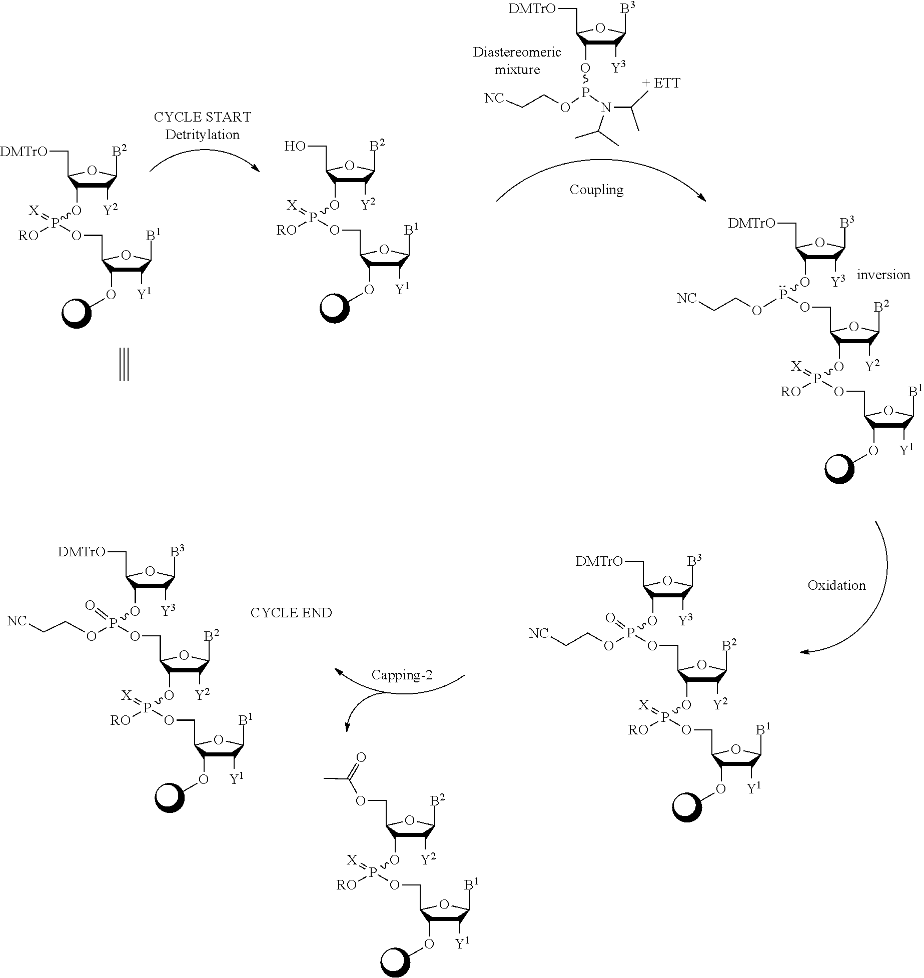

| X | Yⁿ | R | Bⁿ |
|---|---|---|---|
| S | F | (L)-DPSE-Ac | A^{Bz} |
| S | F | (L)-DPSE-Ac | G^{iBu} |
| S | F | (L)-DPSE-Ac | C^{Ac} |
| S | F | (L)-DPSE-Ac | U |
| S | OMe | (L)-DPSE-Ac | A^{Bz} |
| N | F | (L)-PSM-Ac | G^{iBu} |
| N | F | (L)-PSM-Ac | C^{Ac} |
| O | OMe | CNE | A^{Bz} |
| O | OMe | CNE | G^{iBu} |

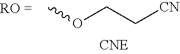

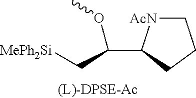

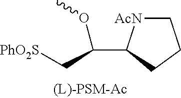

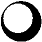

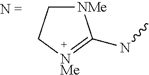

The steps to manufacture WVE-N531 drug substance are described in further detail below.

### Stage 1: Synthesis

Synthesis of the oligonucleotide is carried out on Controlled Pore Glass (CPG) solid support functionalized with 5′-ODMTr-2′-F-dC(N4-Ac), on an automated oligonucleotide synthesizer. All reactions take place on the solid support packed in a column.

### 1. Detritylation

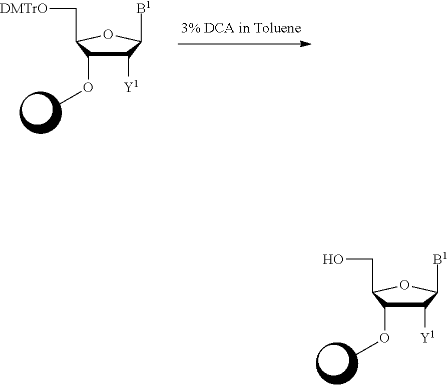

To initiate the synthesis, the CPG-5′-ODMTr-2′-F-dC(N4-Ac) solid support is subjected to acid-catalyzed removal of the DMTr protecting group from the 5′-hydroxyl by treatment with 3% dichloroacetic acid (DCA) in toluene. Complete DMTr removal is ensured by inline UV monitoring based on a watch command in the synthesis program. The DMTr removal is performed in the same way at the beginning of each synthesis cycle and after the final cycle. In every case, following detritylation, the support-bound material is washed with acetonitrile in preparation for the next step of the synthesis.

### 2. Coupling

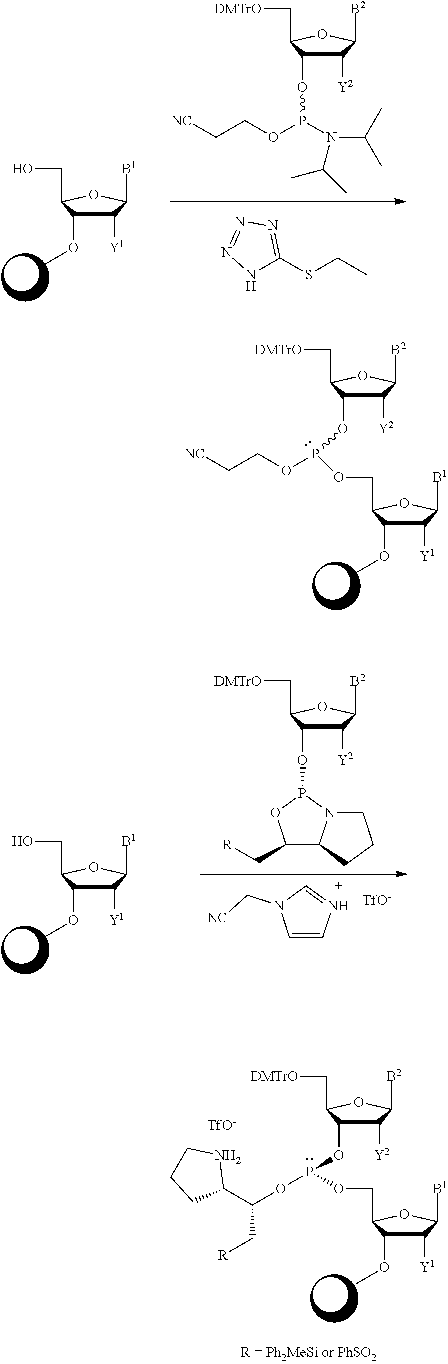

Elongation of the growing oligonucleotide chain is achieved by reaction of the 5′-hydroxyl group of the support-bound oligonucleotide with an excess of a solution of the protected phosphoramidite, in the presence of an activator, either 5-(ethylthio)-1H-tetrazole (ETT) or 1-cyanomethyl imidazolium triflate salt (CMIMT) dissolved in acetonitrile. The phosphoramidites are dissolved in acetonitrile or an 80:20 v/v acetonitrile:isobutyronitrile mixture. The phosphoramidite required in each coupling step is determined by the oligonucleotide sequence. For introduction of phosphate linkage, phosphoramidites and ETT are used. For introduction of a stereodefined phosphorothioate or n001 linkage, (L)-DPSE or (L)-PSM derived phosphoramidite, and CMIMT are used. In either case, the phosphoramidite/activator solutions are mixed inline, pushed onto the synthesis column, then circulated through the column for appropriate amount of time. Subsequently, excess reagents are removed by flushing the synthesis column with acetonitrile.

### 3. Capping-1

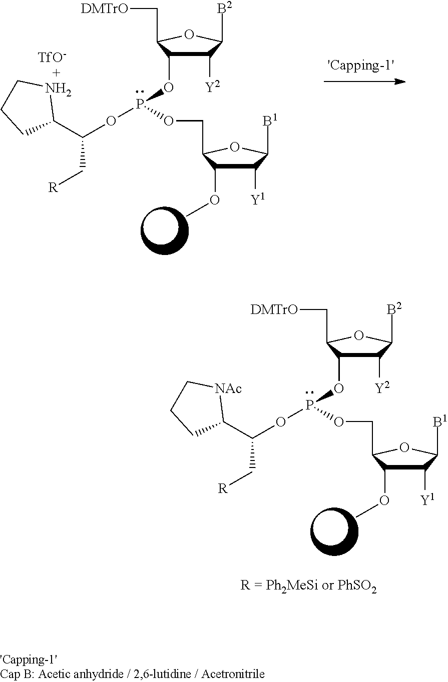

Capping 1 can protect a secondary amine which results from a oxazaphospholidine ring opening on an auxiliary group during a coupling step rendering it unreactive for the remainder of the synthesis. The secondary amine is capped by flowing capping reagent B ([Cap B]: acetic anhydride/2,6-lutidine/acetonitrile (20:30:50, v:v:v) through the synthesis column. Excess reagents are flushed from the synthesis column with acetonitrile.

### 4. Oxidation, Thiolation or Imidation

A newly created P(III) phosphite triester linkage is treated in one of three ways:
1. Oxidation by treatment with iodine in pyridine:water (90:10, v:v), resulting in generation of a P(V) phosphodiester linkage.

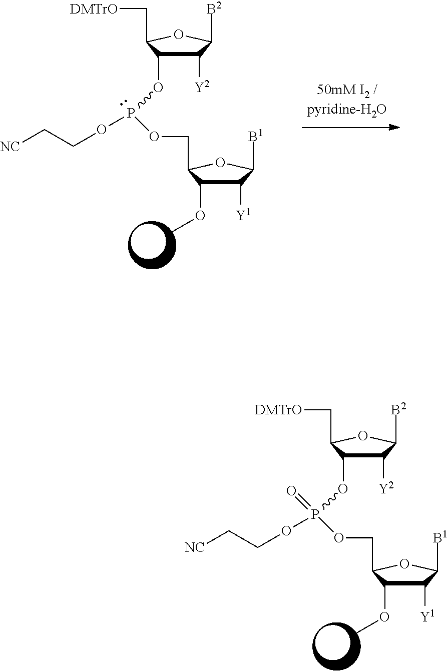

2. Thiolation by treatment with xanthane hydride (XH) in pyridine:acetonitrile (1:1,v:v) resulting in the generation of a stereodefined P(V) phosphorothioate triester linkage.

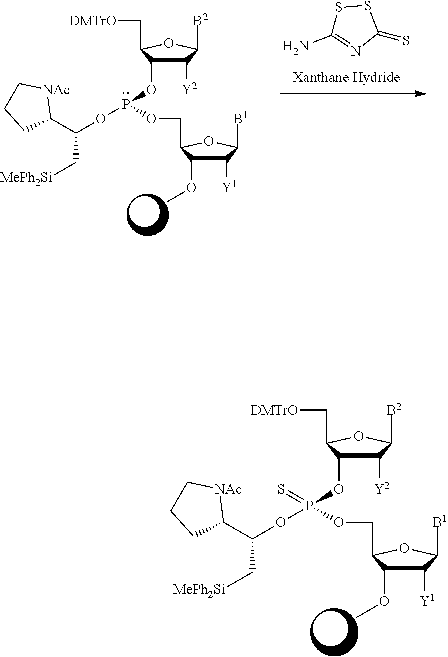

3. Imidation by treatment with 2-azido-1,3-dimethylimidazolinium hexafluorophosphate (ADIH) in acetonitrile resulting in the generation of a stereodefined P(V) phosphorimidate triester linkage.

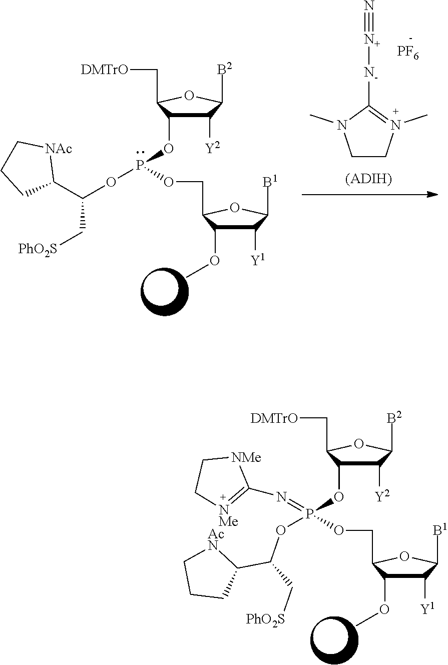

In each case, after the step, excess reagents are flushed from the synthesis column with acetonitrile.

### 5. Capping-2

Typically a coupling reaction proceeds in very high yield but it is not quantitative. A small proportion of the 5′-hydroxy groups, available in any given cycle can fail to couple with an activated phosphoramidite. To prevent reaction during subsequent cycles, these sites are blocked by capping with a mixture of reagents (1:1, v:v) (Capping Reagent A [Cap A]: N-methylimidazole/acetonitrile (20:80,v:v) and Cap B). As a result, 5′-O-acetylated (‘capped’) support-bound oligonucleotide sequences are formed. Excess capping reagents are washed from the column with acetonitrile.

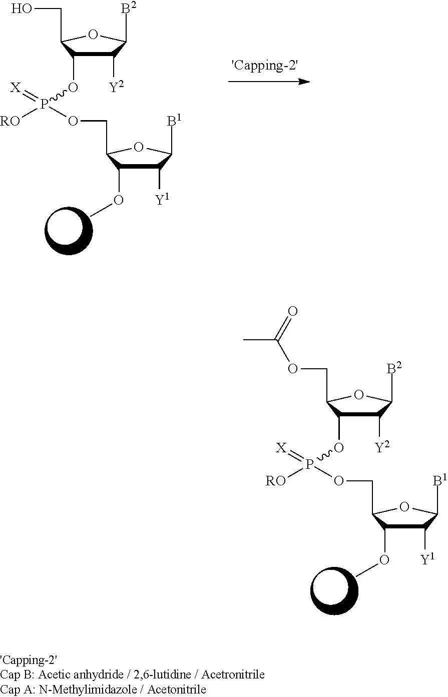

### Oligonucleotide Assembly and Final Step

Independent iteration of the three cycles shown above, using appropriate protected phosphoramidites assemble an entire protected sequence with a DMTr protecting group present at the 5′-terminal position. After addition of the last nucleotide in the sequence, the 5′-terminal DMTr group is removed during a final detritylation step.

### Stage 2: Cleavage and Deprotection

Removal of phosphate protecting groups, and (L)-PSM and (L)-DPSE chiral auxiliaries from stereodefined phosphorimidate triester and phosphorothioate triester, cleavage of the crude oligonucleotide from the solid support, and removal of exocyclic nucleobase protecting groups is performed in a three-step process.

### Step 1: (L)-PSM Chiral Auxiliary and Cyanoethyl Removal with Diethylamine Treatment

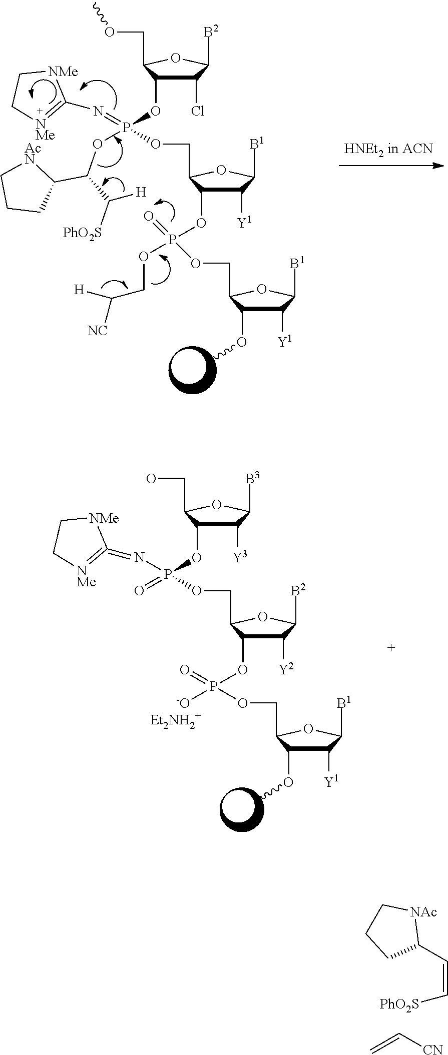

The crude oligonucleotide on the solid support is treated with a solution of diethylamine in ACN which can simultaneously remove cyanoethyl protecting groups from the phosphate triester linkages, and (L)-PSM chiral auxiliary from the phosphorimidate triester to generate phosphodiester and phosphoramidate diester linkages, respectively.

### Step 2: (L)-DPSE Chiral Auxiliary Removal with Fluoride Treatment

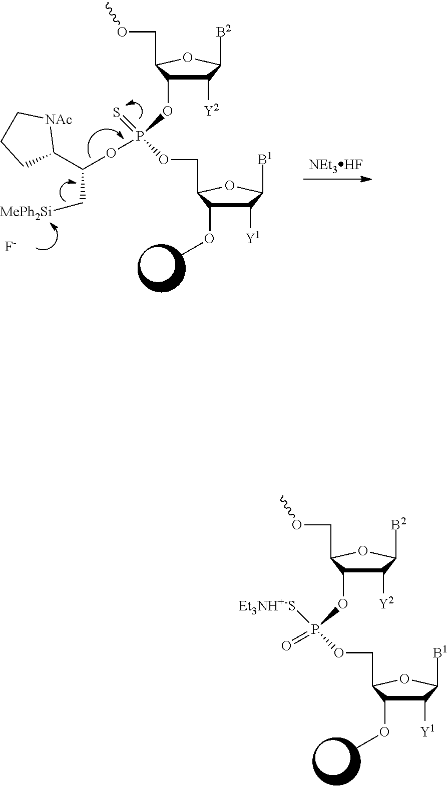

The crude oligonucleotide on the solid support is treated with a temperature-controlled solution of NEt₃·3HF in a mixture of dimethyl sulfoxide (DMSO), NEt₃ and H₂O to effect removal of the chiral auxiliary. This process converts a stereodefined phosphorothioate triester to a stereodefined phosphorothioate diester as shown above

### Step 3: Cleavage and Global Nucleobase Deprotection

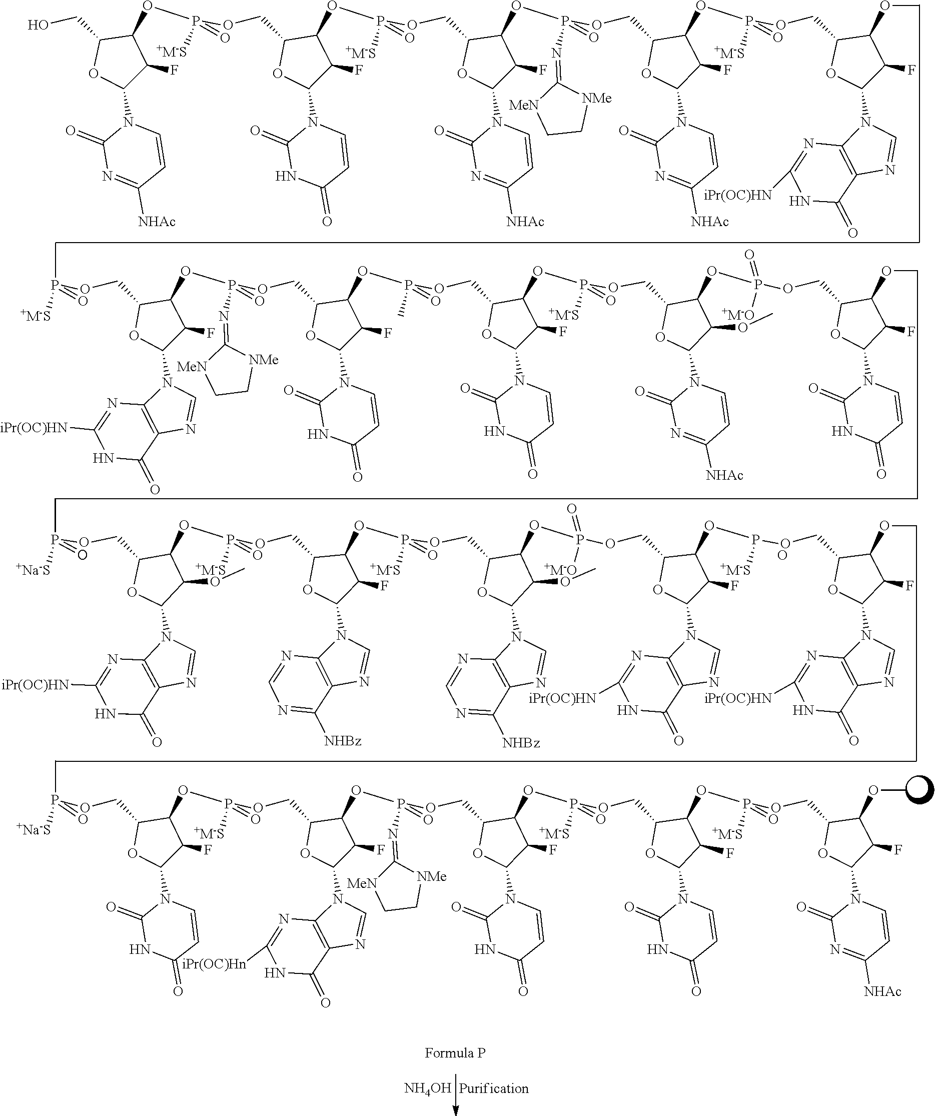

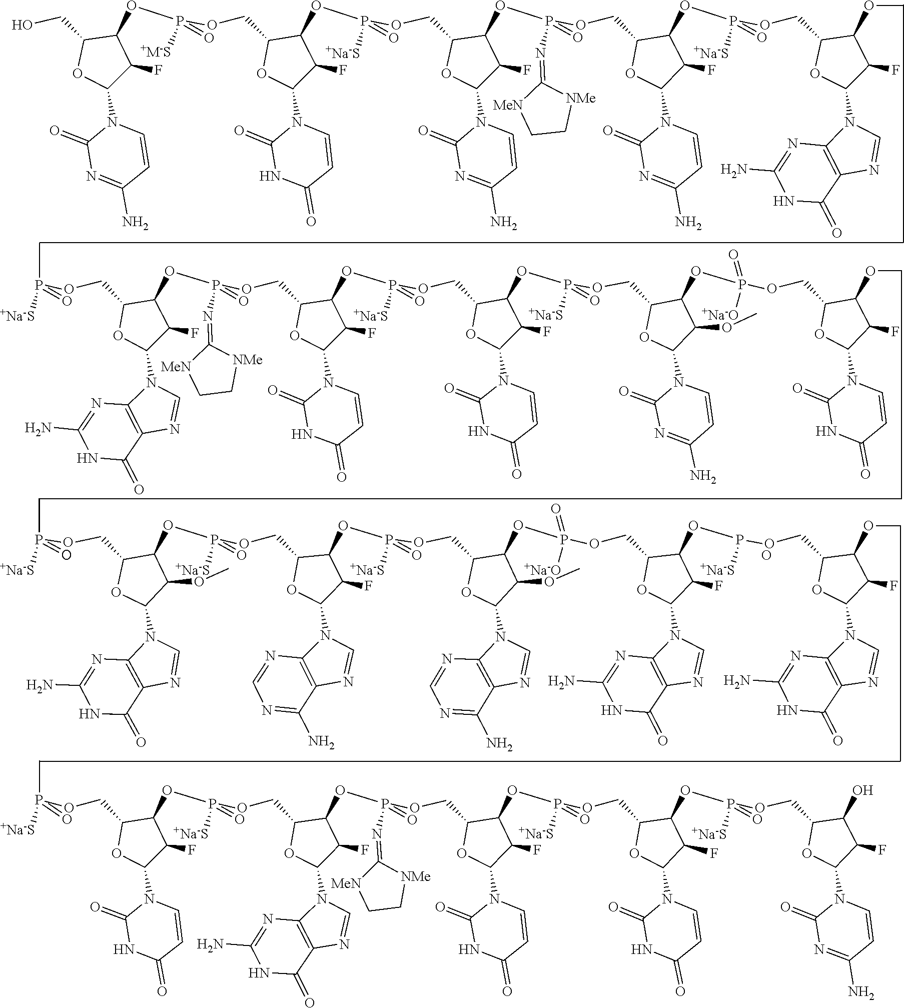

The crude oligonucleotide is then cleaved from the solid support by treatment with ammonium hydroxide in an appropriately sized pressure-rated vessel. This reaction can provide global deprotection of cyanoethyl phosphate groups (may have been deprotected in an earlier step) and exocyclic amino groups (e.g., removal of protecting groups such as acetyl, benzoyl, isobutyryl, etc.). In Formula P, each M⁺ is independently a cation. In some embodiments, each M⁺ is H⁺ (such a compound the “Formula P Acid”). In some embodiments, each M⁺ is independently a cation. In some embodiments, a crude oligonucleotide is a salt of the Formula P Acid. In some embodiments, a crude oligonucleotide composition comprise a salt of the Formula P Acid. In some embodiments, a crude oligonucleotide composition comprise two or more salts of the Formula P Acid.

Typically, after deprotection one or more WVE-N531 salts are provided. In some embodiments, a salt is an ammonium salt. In some embodiments, a deprotection product composition comprises one or more WVE-N531 salts. In some embodiments, a deprotection product composition comprises a WVE-N531 ammonium salt.

### Stage 3: Purification by Anion Exchange Chromatography

Purification of the crude oligonucleotide solution is accomplished by AEX-HPLC. A solution of crude oligonucleotide is loaded onto the purification column packed with TSK-GEL Super Q-5PW media. A purification run is performed using sodium hydroxide buffered eluents. A sodium chloride gradient is used to elute the oligonucleotide from the column. The purification is carried out at ambient temperature. The elution profile is monitored by ultraviolet (UV) spectrophotometry. Fractions are collected and mock pools are evaluated by IP-RP-UPLC. The pool containing oligonucleotide at or above a target purity level is subjected to the next step in the process.

### Stage 4: Concentration and Desalting (Final UF/DF)

The selected fraction pool is then concentrated and diafiltered against purified water to remove the purification buffer by tangential flow filtration (TFF) using regenerated cellulose membrane cassettes. The pool is concentrated, and pH adjusted to 6.5-7.5 with hydrochloric acid or sodium hydroxide. The concentrated oligonucleotide is diafiltered against purified water and concentrated to a target of 1200-1400 OD/mL prior to collecting the desalted oligonucleotide. The system is flushed with purified water to maximize yield combining the concentrated desalted oligonucleotide with the rinses to a target final desalt concentration of 878±68 OD/mL (32.5±2.5 mg/mL).

### Stage 5: Filtration and Packaging

The oligonucleotide solution is filtered through a 0.2-micron filter before filling into drug substance storage containers. The drug substance is packaged in sterile high-density polyethylene (HDPE) bottles, each of which is labeled and sealed in a Mylar foil pouch and stored at −20° C.

### Starting Materials

Certain useful starting materials are listed below:

| Material (Grade) | Step |
|---|---|
| Acetonitrile (ACN) | Synthesis (Solvent) |
| Capping reagent A: | Synthesis (Capping-2) |
| N-Methylimidazole (NMI) in acetonitrile | |
| (20% NMI) | |
| Capping reagent B: | Synthesis (Capping 1 and 2) |
| Acetic anhydride:2,6-Lutidine:Acetonitrile | |
| (20:30:50) | |
| Dichloroacetic acid in toluene (3%) | Synthesis (Detritylation) |
| 5-(Ethylthio)-1H-tetrazole (ETT) in | Synthesis (Coupling |
| acetonitrile | Activator) |
| 1-(Cyanomethyl)-1H-imidazole triflate | Synthesis (Coupling |
| (CMIMT) | Activator) |
| Xanthane Hydride (XH) | Synthesis (Thiolation) |
| Iodine (0.05M) in Pyridine:water (90:10) | Synthesis (Oxidation) |
| Pyridine | Synthesis (Solvent) |
| Isobutyronitrile (IBN) | Synthesis (Solvent) |
| Nitrogen | Synthesis (Inert gas) |
| Ammonium hydroxide (28%) | Cleavage, Deprotection |
| Triethylamine (TEA) | Cleavage, Deprotection |
| Triethylamine•trihydrofluoride | Cleavage, Deprotection |
| (TEA-3HF), 97% | |
| Dimethyl Sulfoxide (DMSO) | Cleavage, Deprotection |
| Diethylamine (DEA) | Cleavage, Deprotection |
| Purified water | Solvent |
| Sodium chloride | Purification, (Buffer salt) |
| Sodium hydroxide | Purification, Neutralization, |
| | CIP |
| Hydrochloric acid (37%) | pH Neutralization |
| Tosoh TSK-Gel Super Q-5PW | Purification media |

Abbreviations: CoA=Certificate of Analysis; NF=National Formulary; CIP=clean in place; USP=United States Pharmacopoeia; ACS=American Chemical Society

| | Molecular Formula |
|---|---|
| Material | (Average MW) |
| 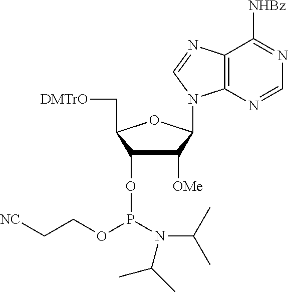 | C₄₈H₅₄N₇O₈P (887.97) |
| 5′-ODMTr-2′-OMe-A(N6-Bz)-CNE phosphoramidite | |
| (mA phosphoramidite) | |
| 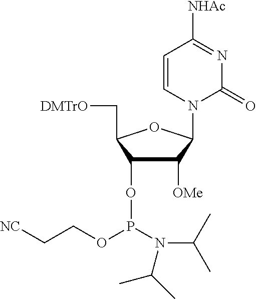 | C₄₂H₅₂N₅O₉P (801.88 Da) |
| 5′-O-DMTr-2′-OMe-C(N4-Ac) CNE phosphoramidite | |
| (mC phosphoramidite) | |
| 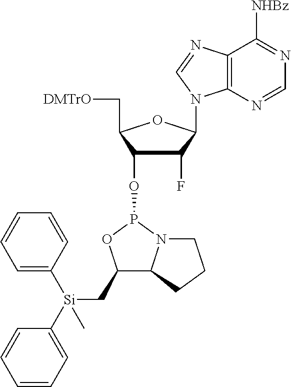 | C₅₇H₅₆FN₆O₇PSi (1015.17) |
| 5′-ODMTr-2′-F-dA(N6-Bz)-(L)-DPSE phosphoramidite | |
| (fA (L) DPSE phosphoramidite) | |
| 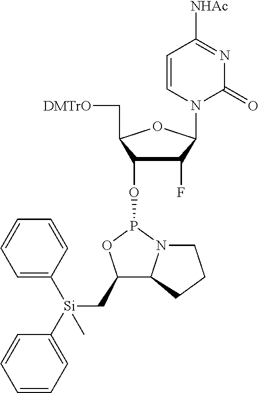 | C₅₁H₅₄FN₄O₈PSi (929.07) |
| 5′-ODMTr-2′-F-dC(N4-Ac)-(L)-DPSE phosphoramidite | |
| (fC (L) DPSE phosphoramidite) | |
| 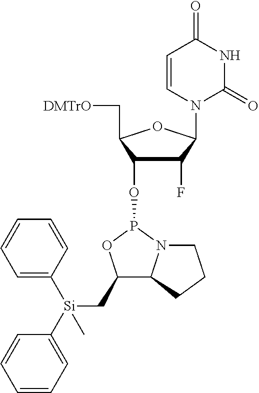 | C₄₉H₅₁FN₃O₈PSi (888.02) |
| 5′-ODMTr-2′-F-dU-(L)-DPSE phosphoramidite | |
| (fU (L) DPSE phosphoramidite) | |
| 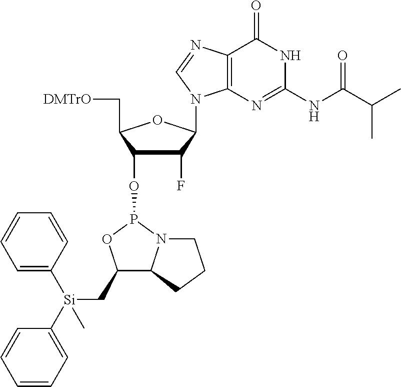 | C₅₄H₅₈FN₆O₈PSi (997.15) |
| 5′-ODMTr-2′-F-dG(N2-iBu)-(L)-DPSE phosphoramidite | |
| (fG (L) DPSE phosphoramidite) | |
| 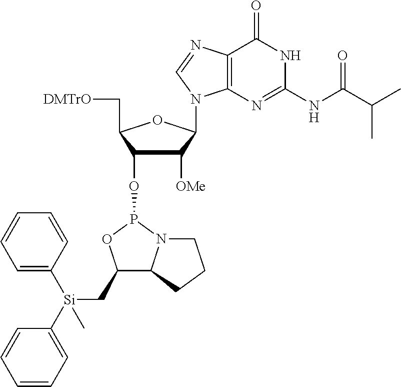 | C₅₅H₆₁N₆O₉PSi (1009.18 Da) |
| 5′-O-DMTr-2′-OMe-G(N2-iBu)-(L)-DPSE phosphoramidite | |
| (mG (L) DPSE phosphoramidite) | |
| 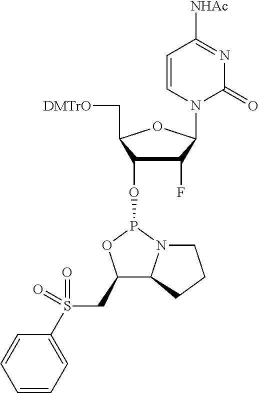 | C₄₄H₄₆FN₄O₁₀PS (872.90 Da) |
| 5′-ODMTr-2′-F-dC(N4-Ac)-(L)-PSM phosphoramidite | |
| (fC (L) PSM phosphoramidite) | |
| 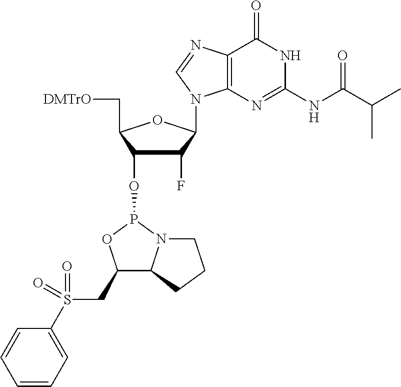 | C₄₇H₅₀FN₆O₁₀PS (940.98 Da) |
| 5′-ODMTr-2′-F-dG(N2-iBu)-(L)-PSM phosphoramidite | |
| (fG (L) PSM phosphoramidite) | |
| Controlled Pore Glass-5′-ODMTr-2′-F-dC(N4-Ac) succinate solid | Solid Support |
| support | |
| 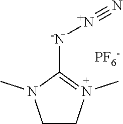 | C₅H₁₀F₆N₅P (285.13 Da) |
| 2-Azido-1,3-dimethylimidazolinium Hexafluorophosphate | |

The starting materials include 9 phosphoramidites, a Controlled Pore Glass (CPG) solid support and 2-azido-1,3-dimethylimidazolinium hexafluorophosphate. The reactive exocyclic groups on nucleobases are appropriately protected to render them unreactive during oligonucleotide synthesis, and the 5′-hydroxy functionality is protected as a 4,4′-dimethoxytrityl ether (DMTr). Phosphoramidites have purity levels of about 90%, about 95% or in some cases about 98% or more (RP-HPLC at about 260 nm (area %) and/or ³¹P NMR by integration), about 9700 or more P(III) purity (by ³¹P NMR integration) and water content of less than 0.2% (w/w). Controlled Pore Glass-5′-ODMTr-2′-F-dC (N4-Ac) succinate solid support is about 120-200 mesh in particle size (analytical sieving), about 540-660 Å in pore diameter (mercury intrusion), about 0.20-0.24 g/cc in density (tap density), ≥about 0.8 cc/g in pore volume (mercury intrusion), ≥about 75 m²/g in surface area Hg intrusion (mercury intrusion), and 70-80 mol/g assessed by DMT ligand assay (spectrophotometric at 498 nm). 2-Azido-1,3-dimethylimidazolinium hexafluorophosphate has a purity of about 98.0% or more (HPLC) and nitrogen content of about 23.70-24.80% (elemental analysis).

Stereochemistry can be established through control of the starting materials for synthesis as well as the synthetic process. Stereodefined phosphoramidites are prepared from appropriately protected nucleosides and a chiral auxiliary, e.g., (L)-DPSE or (L)-PSM. The chemical purity of the stereodefined phosphoramidite can be determined by ³¹P NMR. Configuration can be determined by a combination of ³¹P NMR, ¹H NMR, and ¹³C NMR. As the stereochemical configuration of the protected nucleoside and chiral auxiliaries are fixed, there are 2 possible diastereomers (trans and cis) that can result from the coupling reaction, of which the trans form is predominant, with the cis form being present as a minor impurity.

During the manufacture process, various in-process controls are applied. Upon completion of the synthesis and the cleavage and deprotection stages, the presence of WVE-N531 drug substance in the resulting crude oligonucleotide is identified by LC/MS and its purity quantified by IP-RP-UPLC. During purification, individual fractions as well as selected mock pools are evaluated for purity and impurities by IP-RP-UPLC. Once selected fractions are pooled and concentrated, desalting of the resulting solution is controlled via in-process measurements of conductivity, pH and concentration. In some embodiments, a drug substance or a drug product is manufactured meeting one or more or all of the following criteria. In some embodiments, a drug substance, e.g., WVE-N531 or a salt form thereof, has a certain level of purity as described herein. In some embodiments, WVE-N531 hexadecasodium salt has a certain level of purity as described herein. In some embodiments, WVE-N531 or a salt thereof has a certain level of purity in a composition, e.g., a pharmaceutical composition. In some embodiments, WVE-N531 or a salt thereof has a certain level of purity in a drug product. In some embodiments, WVE-N531 or a salt thereof is WVE-N531 hexadecasodium salt. In some embodiments, WVE-N531 has a level of purity of about 70%-90%, about 70%-85%, about 70%-80%, about 75%-85%, or about 70%, 71%, 72%, 73%, 74%, 75%, 76%, 77%, 78%, 79%, 80%, 81%, 82%, 83%, 84%, 85%, 86%, 87%, 88%, 89%, or 90% or more as determined by IP-RP-UPLC described herein.

### Characterization

Among other things, the present disclosure provides technologies for assessing WVE-N531 or compositions thereof.

Products can be characterized by various technologies. For example, WVE-N531 preparations may be characterized by mass spectrometry. For one preparation, electrospray ionization mass spectrometry (ESI-MS) analysis was performed and the experimentally determined value of 6722 Da is consistent with the theoretical average mass (free acid form) of WVE-N531 drug substance (6723 Da). In some embodiments, the sequence of WVE-N531 or a salt thereof was confirmed by ESI-MS/MS sequencing. For example, in one assessment, at least one sequence-relevant fragment ion was observed for 19 nucleobases in the sequence within 5 ppm error of each calculated (expected) mass. Since the monoisotopic mass of the full-length oligonucleotide was also verified in the ESI-MS/MS analysis, then the identities and locations of all 20 nucleobases have been demonstrated and the sequence was confirmed.

Stereochemical identity comprises the combination of the absolute stereochemical configuration of each chiral linkage phosphorus in an oligonucleotide (Rp or Sp) (for WVE-N531 or a salt thereof, each linkage phosphorus in a phosphorothioate or phosphoramidate group). This combination can give rise to a single diastereomer which is unique among 2ⁿ diastereomers (n=number of chiral linkage phosphorus). In the case of WVE-N531 or a salt thereof, this combination can be illustrated with 5′-SSRSSRSSOSSSOSSSRSS-3′, wherein S, P, and O represent Sp phosphorothioate, Rp N-(1,3-dimethylimidazolidin-2-ylidenyl) phosphoramidate, and phosphate linkages, respectively. WVE-N531 or a salt thereof also comprises 2′-fluoro (F) and 2′-O-Methyl (OMe) modifications.

Establishment of stereochemical identity comprises control of starting materials for synthesis. Synthetic coupling of phosphoramidites prepared from (L)-DPSE or (L)-PSM gives rise to phosphorothioate linkages of Sp chirality and phosphoramidate linkages of Rp chirality respectively, and those prepared from (D)-DPSE or (D)-PSM gives rise to phosphorothioate linkages of Rp chirality and phosphoramidate linkages of Sp chirality. Consistent use of stereodefined phosphoramidites for each coupling reaction allows for control of stereochemical identity of each phosphorothioate or phosphoramidate linkage.

Stereochemical identity can be confirmed using various technologies in accordance with the present disclosure. For example, it can be confirmed by measurement using several different techniques, which in combination, and by comparison with appropriate standards, provide accurate information about, and confirmation of, this attribute. Useful analytical techniques include NMR (¹H, ⁹F, ³¹P, multidimensional, etc.) and enzymatic digestion. In some instances, NMR are performed in phosphate buffer (e.g., pH 7.0). Results including ¹H, ¹⁹F and ³¹P NMR and enzymatic digestion of various preparations are consistent with product structures. In some embodiments, NMR is referenced to water based on DSS standard. Observed data from certain experiments are described below.

³¹P NMR data for a preparation of WVE-N531 drug substance (hexadecasodium salt). Acquired and recorded at 161.98 MHz.

| Chemical Shift (ppm) | Integral | Possible Assignment |
|---|---|---|
| 56.08 | 1 | PS (phosphorothioate) |
| 55.98 | 1 | PS |
| 55.83 | 2 | PS |
| 55.80 | 1 | PS |
| 55.63 | 1 | PS |
| 55.61 | 1 | PS |
| 55.59 | 1 | PS |
| 55.55 | 1 | PS |
| 55.45 | 1 | PS |
| 55.44 | 1 | PS |
| 55.33 | 1 | PS |
| 55.26 | 1 | PS |
| 55.08 | 1 | PS |
| −1.07 | 1 | PO (phosphate) |
| −1.22 | 1 | PO |
| −2.88 | 2 | PN (N-(1,3-dimethylimidazolidin- |
| | | 2-ylidenyl) phosphoramidate) |
| −3.16 | 1 | PN |

¹⁹F NMR data for a preparation of WVE-N531 drug substance (hexadecasodium salt). Acquired and recorded at 348K.

| Chemical Shift (ppm) | Integral |
|---|---|
| −198.82 | 1 |
| −199.36 | 1 |
| −199.42 | 1 |
| −199.48 | 1 |
| −199.58 | 1 |
| −200.27 | 1 |
| −200.59 | 1 |
| −201.53 | 1 |
| −201.68 | 1 |
| −201.99 | 2 |
| −202.01 | 1 |
| −202.41 | 1 |
| −202.44 | 1 |
| −202.46 | 1 |
| −202.57 | 1 |
| −202.87 | 1 |
| −203.14 | 1 |

In some embodiments, a reference standard, e.g., one characterized by NMR, enzymatic digestion, etc. is used to determine stereochemical identity by UPLC as part of batch release. In some embodiments, the provided technology provides reference standards.

FTIR spectrum of solid WVE-N531 hexadecasodium salt as a drug substance was recorded. The major absorbance bands at 1643 cm⁻¹ and 1682 cm⁻¹ are consistent with amide C═O, C═N and C═C stretch vibrations. Broad peaks in the 3200-3300 cm⁻¹ region are also consistent with oligonucleotide functional groups.

Counter ions can be analyzed in accordance with the present disclosure. For example, a sodium content value of 5.1% has been determined by ICP-OES for a preparation of a WVE-N531 drug substance lot, in agreement with the theoretical sodium content value of 5.1% (w/w) (hexadecasodium salt).

A prepared solution of WVE-N531 drug substance is a clear (no visible material and essentially free of particulate matter), colorless solution. The pH of WVE-N531 drug substance in purified water was found to be about 7.3.

The molar extinction coefficient of WVE-N531 drug substance was experimentally determined in water to be 186486 M-‘cm-’ using a lot. Using a molecular weight of 6918.72 g/mol (free acid form), this equates to an absorptivity factor of 27.0 OD/mg.

In various embodiments, impurities are controlled at low levels as described herein. For example, in some embodiments, impurity level is lower than about 30% as described herein.

WVE-N531 drug substance preparations (including associated impurities) were assessed in GLP in vitro genotoxicity studies and in an in vivo micronucleus study. No genotoxicity was observed.

Various preparations of WVE-N531 drug substance have met specified limits for residual solvents, including acetonitrile, toluene and pyridine (ICH Class 2), and elemental impurities, including various elements in ICH Class 1, 2A, 3, etc.

The fidelity of transfer of chiral information (diastereoselectivity) from each stereodefined phosphoramidite to its stereodefined Sp phosphorothioate or Rp N-(1,3-dimethylimidazolidin-2-ylidenyl) phosphoramidate (PN) linkage in WVE-N531 or a salt thereof during oligonucleotide synthesis is very high. The overall stereochemical purity of the oligonucleotide can be represented by a product of the diastereoselectivities of incorporation of each of the 17 stereodefined internucleotide linkages.

In some embodiments, stereopurity is assessed using dimer modeling. The 14 dimer units that represent the composition of the stereodefined phosphorothioate or PN internucleotide linkages within WVE-N531 drug substance (the fGn001RfU, fG*SfG and fU*SfC dimers are each represented twice) were independently synthesized and assessed for stereochemical purity. The stereochemical purity of WVE-N531 or a salt thereof can be estimated as the product of the measured diastereoselectivities of the 17 individual dimers. The synthesis conditions used to produce each dimer were identical to those applied to the WVE-N531 drug substance. Compared to full length WVE-N531 drug substance, these model dimer units are more easily analyzed by standard analytical methods such as IP-RP-UPLC, and both Rp and Sp diastereoisomers are readily separated and quantified by this technique. IP-RP-UPLC data for the model dimers are presented below.

Diastereoselectivity

| Dimer | % Sp | % Rp |
|---|---|---|
| fC*SfU | 98.57 | 1.43 |
| fU*SfC | 98.81 | 1.19 |
| fU*SfC | 98.81 | 1.19 |
| fCn001RfC | 0.69 | 99.31 |
| fGn001RfU | 0.38 | 99.62 |
| fGn001RfU | 0.38 | 99.62 |
| fC*SfG | 98.60 | 1.40 |
| fG*SfG | 98.97 | 1.03 |
| fG*SfG | 98.97 | 1.03 |
| fU*SmC | 99.12 | 0.88 |
| fU*SmG | 98.19 | 1.81 |
| mG*SfA | 99.70 | 0.30 |
| fA*SmA | 98.66 | 1.34 |
| fG*SfU | 99.38 | 0.62 |
| fU*SfG | 99.15 | 0.85 |
| fU*SfU | 99.12 | 0.88 |
| fU*SfU | 99.12 | 0.88 |

The model studies demonstrate excellent stereochemical fidelity at each location, with stereochemical purity values of ≥98% at each location in the sequence. The overall stereochemical purity, based on the product of the stereochemical purity of each individual constituent dimers, is 85% (84.9%=product of the 17 diastereoselectivity fidelity values above).

In some embodiments, the present disclosure provides WVE-N531 with high stereopurity, e.g., as assessed using dimer modeling described herein. In some embodiments, a stereopurity is about 80% or more. In some embodiments, a stereopurity is about 81% or more. In some embodiments, a stereopurity is about 82% or more. In some embodiments, a stereopurity is about 83% or more. In some embodiments, a stereopurity is about 84% or more. In some embodiments, a stereopurity is about 85% or more. In some embodiments, a stereopurity is about 86% or more. In some embodiments, a stereopurity is about 87% or more. In some embodiments, a stereopurity is about 88% or more. In some embodiments, a stereopurity is about 89% or more. In some embodiments, a stereopurity is about 90% or more. In some embodiments, a stereopurity is about 80%-90%. In some embodiments, a stereopurity is about 83%-87%.

Release specifications of WVE-N531 drug substance may include one or more specifications described herein, e.g., appearance (e.g., visual), sequence identity (e.g., by MS/MS), molecular weight, purity (e.g., area % by IP-RP-UPLC), impurities (e.g., area % by IP-RP-UPLC), sodium content (e.g., by ICP-OES, etc.), concentration (free acid form) (e.g., by UV spectrophotometry), pH (e.g., of solution in purified water), residual solvents (e.g., by gas chromatography), elemental impurities (e.g., by ICP-MS), stereochemical identity (e.g., by UPLC), bacterial endotoxins (e.g., by USP <85>, Ph. Eur 2.6.14), bioburden (total microbial aerobic count, total yeast and bolds count, etc. by USP <61) and/or Ph. Eur 2.6.12), etc.

Useful parameters for identity (molecular weight) by LC MS are described below:

| Parameter | Value | | |
|---|---|---|---|
| Column | Waters Acquity BEH C18 Column, 1.7 μm, 2.1 × | | |
| | 50 mm, or equivalent | | |
| Column Temperature | 55° C. | | |
| Mobile Phase A | 100 mM HFIP, 10 mM TEA in water | | |
| Mobile Phase B | Acetonitrile | | |
| | Time (Min) | % MPA | % MPB |
| Gradient | Initial | 99.0 | 1.0 |
| | 2.00 | 99.0 | 1.0 |
| | 11.00 | 85.0 | 15.0 |
| | 13.00 | 50.0 | 50.0 |
| | 14.00 | 50.0 | 50.0 |
| | 14.10 | 99.0 | 1.0 |
| | 15.00 | 99.0 | 1.0 |

| | |
|---|---|
| Run Time | 15 min |
| Injection Volume | 2 μL |
| Flow Rate | 0.8 mL/min |
| Detection Wavelength | 260 nm |

| | Parameter | Value |
|---|---|---|
| MS Acquisition | Scan Type | Full MS Scan |
| Parameters | Scan Range (m/z) | 500-3000 |
| | Polarity | Negative |

In some embodiments, system suitability is confirmed when the molecular weight (deconvoluted mass) for full length product (FLP) for 3 initial injections of the analytical reference material should be 6923±3 Da for WVE-N531; in addition, blank chromatogram has no interfering peaks other than the solvent front and gradient shift.

The identity of WVE-N531 or a salt thereof can be determined by liquid chromatography mass spectrometry (LC-MS). Samples are prepared in water and injected on a Waters Acquity BEH C18 column. Analysis employs a gradient of mobile phase A (hexafluoroisopropanol [HFIP] and triethylamine [TEA] in water) and mobile phase B (acetonitrile). A summary of method parameters is provided below as an example.

| Parameter | Value | | |
|---|---|---|---|
| Column | Flow Injection | | |
| Mobile Phase A | 400 mM HFIP, 15 mM TEA in water | | |
| Mobile Phase B | Acetonitrile | | |
| | Time (Min) | % MPA | % MPB |
| Isocratic Mobile | 1 minute | 70.0 | 30 |
| Phase Composition | | | |

| | |
|---|---|
| Injection Rate | 5 μL/min (using syringe) |
| Flow Rate | 0.3 mL/min |
| Detection Wavelength | 260 nm |

| | Parameter | Value |
|---|---|---|
| MS | Scan Type | Full MS Scan |
| Acquisition | Scan Range (m/z) | 100-3000 |
| Parameters | Polarity | Negative |
| | Spray Voltage (kV) | 2.7 |
| | Capillary Temp (° C.) | 280 |
| | Acceptable parent | 6918.7251 ± 0.1 Da |
| | exact mass | |
| MS/MS | MS/MS Scan Type | Targeted Fragmentation Scan |
| Acquisition | Polarity | Negative |
| Parameters | Scan Range (Da) | 200-3000 |
| | Fragment ion selected | Charge State m/z 864 ± 1.5 |
| | | Da (z = −5) |
| | Fragment ion selected | Charge State m/z 2306 ± 1.5 |
| | | Da (z = −3) |

In some embodiments, the present disclosure provides technologies for assessing WVE-N531 purity and/or impurities. In some embodiments, purity and impurities of WVE-N531 can be determined by ion-pair reversed-phase UPLC (IP-RP-UPLC), e.g., using a Waters BEH C1 Column. A useful procedure is described below as an example (Protocol A). The separation is accomplished using a gradient of mobile phase A (hexafluoroisopropanol [HFIP] and diisopropylethylamine [DIPEA] in water) and mobile phase B (30% acetonitrile in water). A useful set of parameters are provided below (Set A) and have been utilized to determine levels of purity/impurity for various preparations described herein. Various impurities including diastereomer impurities can be separated using this method (of the 17 W VE-N531 diastereomers each of which only differs from WVE-N531 at a single linkage phosphorus (DS1-DS17), 6 diastereomers are completely resolved and 9 are partially resolved). In some embodiments, the present disclosure provides a method for characterizing WVE-N531 or a salt thereof, or a composition comprising WVE-N531 or a salt thereof, comprising HPLC or UPLC analysis comprising one or more or all of the parameters (e.g., column, one or more solvents in Mobile Phase A, one or more solvents in Mobile Phase B, a mobile phase comprising one or more or all solvents in Mobile Phase A, a mobile phase comprising one or more or all solvents in Mobile Phase B, Mobile Phase A, Mobile phase B, flow rate, un time, gradient, etc.).

| Parameter | Value | | | |
|---|---|---|---|---|
| Column | Waters BEH Oligonucleotide C18 Column, | | | |
| | 1.7 μm, 2.1 × 150 mm, or equivalent | | | |
| Mobile Phase A | 0.5% HFIP, 0.2% DIPEA, 10% Acetonitrile | | | |
| | in water | | | |
| Mobile Phase B | 30% Acetonitrile in water | | | |
| Injection Volume | 2 μL | | | |
| Flow Rate | 0.3 mL/min | | | |
| Run Time | 22 minutes | | | |
| Detection Wavelength | 260 nm | | | |
| | Time | % A | % B | Curve |
| Gradient | Initial | 86 | 14 | N/A |
| | 4 | 78 | 22 | 6 |
| | 11 | 78 | 22 | 6 |
| | 15 | 15 | 85 | 6 |
| | 15.1 | 86 | 14 | 1 |
| | 22 | 86 | 14 | 1 |

| Diastereomer | Description |
|---|---|
| DS1 | fC*RfU*SfCn001RfC*SfG*SfGn001RfU*SfU*SmCfU*SmG*SfA*SmAfG*SfG*SfU*SfGn001RfU*SfU*SfC |
| DS2 | fC*SfU*RfCn001RfC*SfG*SfGn001RfU*SfU*SmCfU*SmG*SfA*SmAfG*SfG*SfU*SfGn001RfU*SfU*SfC |
| DS3 | fC*SfU*SfCn001.SfC*SfG*SfGn001RfU*SfU*SmCfU*SmG*SfA*SmAfG*SfG*SfU*SfGn001RfU*SfU*SfC |
| DS4 | fC*SfU*SfCn001RfC*RfG*SfGn001RfU*SfU*SmCfU*SmG*SfA*SmAfG*SfG*SfU*SfGn001RfU*SfU*SfC |
| DS5 | fC*SfU*SfCn001RfC*SfG*RfGn001RfU*SfU*SmCfU*SmG*SfA*SmAfG*SfG*SfU*SfGn001RfU*SfU*SfC |
| DS6 | fC*SfU*SfCn001RfC*SfG*SfGn001SfU*SfU*SmCfU*SmG*SfA*SmAfG*SfG*SfU*SfGn001RfU*SfU*SfC |
| DS7 | fC*SfU*SfCn001RfC*SfG*SfGn001RfU*RfU*SmCfU*SmG*SfA*SmAfG*SfG*SfU*SfGn001RfU*SfU*SfC |
| DS8 | fC*SfU*SfCn001RfC*SfG*SfGn001RfU*SfU*RmCfU*SmG*SfA*SmAfG*SfG*SfU*SfGn001RfU*SfU*SfC |
| DS9 | fC*SfU*SfCn001RfC*SfG*SfGn001RfU*SfU*SmCfU*RmG*SfA*SmAfG*SfG*SfU*SfGn001RfU*SfU*SfC |
| DS10 | fC*SfU*SfCn001RfC*SfG*SfGn001RfU*SfU*SmCfU*SmG*RfA*SmAfG*SfG*SfU*SfGn001RfU*SfU*SfC |
| DS11 | fC*SfU*SfCn001RfC*SfG*SfGn001RfU*SfU*SmCfU*SmG*SfA*RmAfG*SfG*SfU*SfGn001RfU*SfU*SfC |
| DS12 | fC*SfU*SfCn001RfC*SfG*SfGn001RfU*SfU*SmCfU*SmG*SfA*SmAfG*RfG*SfU*SfGn001RfU* SfU*SfC |
| DS13 | fC*SfU*SfCn001RfC*SfG*SfGn001RfU*SfU*SmCfU*SmG*SfA*SmAfG*SfG*RfU*SfGn001RfU*SfU*SfC |
| DS14 | fC*SfU*SfCn001RfC*SfG*SfGn001RfU*SfU*SmCfU*SmG*SfA*SmAfG*SfG*SfU*RfGn001RfU*SfU*SfC |
| DS15 | fC*SfU*SfCn001RfC*SfG*SfGn001RfU*SfU*SmCfU*SmG*SfA*SmAfG*SfG*SfU*SfGn001SfU*SfU*SfC |
| DS16 | fC*SfU*SfCn001RfC*SfG*SfGn001RfU*SfU*SmCfU*SmG*SfA*SmAfG*SfG*SfU*SfGn001RfU*RfU*SfC |
| DS17 | fC*SfU*SfCn001RfC*SfG*SfGn001RfU*SfU*SmCfU*SmG*SfA*SmAfG*SfG*SfU*SfGn001RfU*SfU*RfC |

In some embodiments, all individual impurities ≥0.10 area %, as a function of relative retention time to the main peak, as well as total of all impurities ≥0.10 area % are included in analysis. In some embodiments, WVE-N531 identity is confirmed by comparing relative retention time with system suitability standard.

In some embodiments, the present disclosure provide technologies for confirming stereochemical identity of WVE-N531 or a diastereomer thereof with respect to one or more linkage phosphorus. In some embodiments, stereochemical identity of WVE-N531 is determined by IP-RP-UPLC using a Waters BEH C18 Column. A useful procedure is described below as an example (Protocol B). The separation is accomplished using a gradient of mobile phase A (hexafluoroisopropanol [HFIP] and DIPEA in water) and mobile phase B (30% acetonitrile in water). A summary of method parameters (Set B) is provided below. In some embodiments, the present disclosure provides a method for characterizing WVE-N531 or a salt thereof, or a composition comprising WVE-N531 or a salt thereof, comprising HPLC or UPLC analysis comprising one or more or all of the parameters (e.g., column, one or more solvents in Mobile Phase A, one or more solvents in Mobile Phase B, a mobile phase comprising one or more or all solvents in Mobile Phase A, a mobile phase comprising one or more or all solvents in Mobile Phase B, Mobile Phase A, Mobile phase B, flow rate, run time, gradient, etc.).

| Parameter | Value | | | |
|---|---|---|---|---|
| Column | Waters BEH Oligonucleotide C18 Column, | | | |
| | 1.7 μm, 2.1 × 150 mm, or equivalent | | | |
| Mobile Phase A | 0.3% HFIP, 0.18% DIPEA, 10% Acetonitrile | | | |
| | in water | | | |
| Mobile Phase B | 30% Acetonitrile in water | | | |
| Injection Volume | 2 μL | | | |
| Flow Rate | 0.2 mL/min | | | |
| Run Time | 30 minutes | | | |
| Column Temperature | 45° C. | | | |
| Detection Wavelength | 260 nm | | | |
| | Time | % A | % B | Curve |
| Gradient | Initial | 86 | 14 | N/A |
| | 1 | 86 | 14 | 6 |
| | 3 | 76 | 24 | 6 |
| | 10 | 75 | 25 | 6 |
| | 17 | 65 | 35 | 6 |
| | 20 | 15 | 85 | 6 |
| | 22 | 15 | 85 | 6 |
| | 23 | 86 | 14 | 1 |
| | 30 | 86 | 14 | 1 |

Among other things, the present disclosure provides WVE-N531 of high purity as described herein. In some embodiments, the present disclosure provides WVE-N531 of high stereochemical purity, e.g., as determined utilizing IP-RP-UPLC with parameters of Set A and/or Set B. In some embodiments, the present disclosure provides compositions comprising one or more (“the first group”) of WVE-N531 and DS1 to DS17, none of one or more (“the second group) of WVE-N531 and DS1 to DS17, and optionally reduced levels of one or more (“the third group”) of WVE-N531 and DS1 to DS17, wherein each reduced level is independently lower than (e.g., about or no more than about 90%, 80%, 70%, 60%, 50%, 40%, 30%, 20% or 10% of, in some embodiments, about or no more than about 50% of) one or more or each of the levels of the first group members. In some embodiments, the present disclosure provides a composition comprising one of WVE-N531 and DS1 to DS17, wherein the composition is free of one or more (e.g., 1, 2, 3, 4, 5, 6, 7, 8, 9, 10, 11, 12, 13, 14, 15, 16 or 17) of the rest of WVE-N531 and DS1 to DS17. In some embodiments, a composition is free of one of the rest of WVE-N531 and DS1 to DS17. In some embodiments, a composition is free of two of the rest of WVE-N531 and DS1 to DS17. In some embodiments, a composition is free of three of the rest of WVE-N531 and DS1 to DS17. In some embodiments, a composition is free of four of the rest of WVE-N531 and DS1 to DS17. In some embodiments, a composition is free of five of the rest of WVE-N531 and DS1 to DS17. In some embodiments, a composition is free of six of the rest of WVE-N531 and DS1 to DS17. In some embodiments, a composition is free of seven of the rest of WVE-N531 and DS1 to DS17. In some embodiments, a composition is free of eight of the rest of WVE-N531 and DS1 to DS17. In some embodiments, a composition is free of nine of the rest of WVE-N531 and DS1 to DS17. In some embodiments, a composition is free of 10 of the rest of WVE-N531 and DS1 to DS17. In some embodiments, a composition is free of 11 of the rest of WVE-N531 and DS1 to DS17. In some embodiments, a composition is free of 12 of the rest of WVE-N531 and DS1 to DS17. In some embodiments, a composition is free of 13 of the rest of WVE-N531 and DS1 to DS17. In some embodiments, a composition is free of 14 of the rest of WVE-N531 and DS1 to DS17. In some embodiments, a composition is free of 15 of the rest of WVE-N531 and DS1 to DS17. In some embodiments, a composition is free of 16 of the rest of WVE-N531 and DS1 to DS17. In some embodiments, a composition is free of 17 of the rest of WVE-N531 and DS1 to DS17. In some embodiments, a composition comprises WVE-N531. In some embodiments, a composition comprises DS1. In some embodiments, a composition comprises DS2. In some embodiments, a composition comprises DS3. In some embodiments, a composition comprises DS4. In some embodiments, a composition comprises DS5. In some embodiments, a composition comprises DS6. In some embodiments, a composition comprises DS7. In some embodiments, a composition comprises DS8. In some embodiments, a composition comprises DS9. In some embodiments, a composition comprises DS10. In some embodiments, a composition comprises DS11. In some embodiments, a composition comprises DS12. In some embodiments, a composition comprises DS13. In some embodiments, a composition comprises DS14. In some embodiments, a composition comprises DS15. In some embodiments, a composition comprises DS16. In some embodiments, a composition comprises DS17. In some embodiments, at least WVE-N531 is absent from a composition. In some embodiments, at least DS1 is absent from a composition. In some embodiments, at least DS2 is absent from a composition. In some embodiments, at least DS3 is absent from a composition. In some embodiments, at least DS4 is absent from a composition. In some embodiments, at least DS5 is absent from a composition. In some embodiments, at least DS6 is absent from a composition. In some embodiments, at least DS7 is absent from a composition. In some embodiments, at least DS8 is absent from a composition. In some embodiments, at least DS9 is absent from a composition. In some embodiments, at least DS10 is absent from a composition. In some embodiments, at least DS11 is absent from a composition. In some embodiments, at least DS12 is absent from a composition. In some embodiments, at least DS13 is absent from a composition. In some embodiments, at least DS14 is absent from a composition. In some embodiments, at least DS15 is absent from a composition. In some embodiments, at least DS16 is absent from a composition. In some embodiments, at least DS17 is absent from a composition. In some embodiments, the present disclosure provides technologies for preparing such compositions, e.g., IP-RP-UPLC technologies described herein (e.g., those that can fully or partially resolve diastereomers). For example, in some embodiments, when a peak for one of WVE-N531 and DS1 to DS17 is fully resolved from one or more peaks of one or more of WVE-N531 and DS1 to DS17, the peak represents a composition comprising the one of WVE-N531 and DS1 to DS17 that is free of the one or more of WVE-N531 and DS1 to DS17. In some embodiments, presence, absence and/or levels are assessed using provided technologies, e.g., IP-RP-UPLC as described herein.

In some embodiments, the present disclosure provides a composition comprising WVE-N531, wherein the composition is free of one or more (e.g., 1, 2, 3, 4, 5, 6, 7, 8, 9, 10, 11, 12, 13, 14, 15, 16 or 17) of DS1 to DS17. In some embodiments, a composition is free of one of DS1 to DS17. In some embodiments, a composition is free of two of DS1 to DS17. In some embodiments, a composition is free of three of DS1 to DS17. In some embodiments, a composition is free of four of DS1 to DS17. In some embodiments, a composition is free of five of DS1 to DS17. In some embodiments, a composition is free of six of DS1 to DS17. In some embodiments, a composition is free of seven of DS1 to DS17. In some embodiments, a composition is free of eight of DS1 to DS17. In some embodiments, a composition is free of nine of DS1 to DS17. In some embodiments, a composition is free of 10 of DS1 to DS17. In some embodiments, a composition is free of 11 of DS1 to DS17. In some embodiments, a composition is free of 12 of DS1 to DS17. In some embodiments, a composition is free of 13 of DS1 to DS17. In some embodiments, a composition is free of 14 of DS1 to DS17. In some embodiments, a composition is free of 15 of DS1 to DS17. In some embodiments, a composition is free of 16 of DS1 to DS17. In some embodiments, a composition is free of 17 of DS1 to DS17. In some embodiments, at least DS1 is absent from a composition. In some embodiments, at least DS2 is absent from a composition. In some embodiments, at least DS3 is absent from a composition. In some embodiments, at least DS4 is absent from a composition. In some embodiments, at least DS5 is absent from a composition. In some embodiments, at least DS6 is absent from a composition. In some embodiments, at least DS7 is absent from a composition. In some embodiments, at least DS8 is absent from a composition. In some embodiments, at least DS9 is absent from a composition. In some embodiments, at least DS10 is absent from a composition. In some embodiments, at least DS11 is absent from a composition. In some embodiments, at least DS12 is absent from a composition. In some embodiments, at least DS13 is absent from a composition. In some embodiments, at least DS14 is absent from a composition. In some embodiments, at least DS15 is absent from a composition. In some embodiments, at least DS16 is absent from a composition. In some embodiments, at least DS17 is absent from a composition.

Similar to WVE-N531, each of DS1 to DS17 may independently be in various forms. In some embodiments, in a composition it is in one form; in some embodiments, it is in two or more forms. In some embodiments, a form is a salt form. In some embodiments, a form is a pharmaceutically acceptable salt form. In some embodiments, each form is independently a salt form. In some embodiments, each form is independently a pharmaceutically acceptable salt form. In some embodiments, a form is hexadecasodium salt. In some embodiments, in a liquid composition, e.g., an optionally buffered solution, WVE-N531 and/or D1 to DS17 are dissolved and may dissociate to yield cations and anions.

The liquid WVE-N531 drug substance is packaged in sterile high density polyethylene (HDPE) bottles with polypropylene screw closures, labeled and sealed in a Mylar foil pouches which provide a gas/moisture barrier with high levels of abrasion and puncture resistance.

Various batches of WVE-N531 hexadecasodium salt have been manufactured as drug substance, in some instances, at about 20 mmol scale (e.g., in one instance, 2×23 mmol scale). Certain preparations was utilized in nonclinical studies including GLP toxicology studies. Certain preparations were manufactured for clinical studies. Manufactured products are stable: No significant changes in stability indicating attributes have been observed for GMP drug substance of multiple lots after 24 months of storage at the long-term storage condition of −20° C.±5° C. and after 14 days of storage at an accelerated condition of 5° C.±3° C.

### Drug Product

In some embodiments, the present disclosure provides a pharmaceutical composition comprising or delivering WVE-N531 or a pharmaceutically acceptable salt form thereof and a pharmaceutically acceptable carrier. In some embodiments, the present disclosure provides a pharmaceutical composition comprising WVE-N531 hexadecasodium salt and a pharmaceutically acceptable carrier. In some embodiments, the present disclosure provides a WVE-N531 drug product. In some embodiments, a WVE-N531 drug product comprises a WVE-N531 drug substance, e.g., WVE-N531 hexadecasodium salt manufactured using a process described above.

In some embodiments, a pharmaceutically acceptable carrier is or comprises a buffered solution. In some embodiments, a pharmaceutically acceptable carrier is or comprises a phosphate buffered solution. In some embodiments, a pharmaceutically acceptable carrier is or comprises an isotonic phosphate buffered solution. In some embodiments, a WVE-N531 drug product is or comprises a WVE-N531 drug substance in an isotonic phosphate buffered solution. In some embodiments, a pharmaceutical composition or a drug product that closely matches physiological conditions in blood with respect to pH and tonicity. In some embodiments, it is for intravenous (IV) administration.

Certain WVE-N531 drug products and useful manufacturing technologies are described below as examples.

In some embodiments, a WVE-N531 pharmaceutical composition, e.g., a drug product is packaged in a 10 mL USP Type 1 clear glass vial capped with a FluroTec® coated chlorobutyl rubber stopper and an aluminum overseal with a white flip-off cap. In some embodiments, each drug product vial contains WVE-N531 drug substance equivalent to 36 mg WVE-N531 free acid form (6 mg/mL; nominal content 36 mg, total extractable volume 6 mL). In some embodiments, the components of a WVE-N531 drug product comprise or are WVE-N531 drug substance, potassium phosphate monobasic, sodium phosphate dibasic, and sodium chloride in water for injection (WFI). Sodium hydroxide and/or hydrochloric acid may also be used to adjust the pH of the formulated solution into the required range.

An example composition of WVE-N531 drug product is presented below:

| | | | Total Amount | Concentration |
|---|---|---|---|---|
| Component | Quality Standard | Function | Per Vial | (mg/mL) |
| WVE-N531 Drug | In-house | Active | 36 mg (free | 6 mg/mL (free |
| Substance | | Substance | acid form) | acid form) |
| Potassium Phosphate, | NF, Ph. Eur., BP | Buffer | 1.062 mg | 0.177 |
| Monobasic, Crystal | | | | |
| Sodium Phosphate, | USP, Ph. Eur., JP | Buffer | 7.326 mg | 1.221 |
| Dibasic, Anhydrous | | | | |
| Sodium Chloride, Granular | USP, Ph. Eur., JP, BP | Tonicificr | 49.788 mg | 8.298 |
| Hydrochloric Acid | NF, Ph. Eur., JP, BP | pH | as needed | N/A |
| | | adjustment | | |
| Sodium Hydroxide, Pellets | NF, Ph. Eur., JP, BP | pH | as needed | N/A |
| | | adjustment | | |
| Water for Injection | USP, Ph. Eur. | Diluent | qs to achieve | N/A |
| | | | target fill | |
| | | | volume | |

Abbreviations: USP=United States Pharmacopeia, NF=National Formulary, Ph. Eur.=European Pharmacopoeia, JP=Japanese Pharmacopoeia, BP=British Pharmacopoeia; qs=quantity sufficient, N/A=Not Applicable

In some embodiments, a WVE-N531 drug product is a colorless solution with no visible material and essentially free of particulates. In some embodiments, pH of a 6 mg/mL solution in phosphate buffered saline solution is 7.0-7.8 with osmolality in the range of 280-320 mOsm/kg. In some embodiments, density of WVE-N531 drug product is 1.0 g/mL.

A WVE-N531 drug product lot was manufactured at a concentration of 36 mg/vial (free acid form). The 36 mg strength is achieved by filling 6.35 mL of a 6 mg/mL formulation which included a 0.35 mL overfill above the nominal volume of 6 mL. All material contact components were sterilized prior to use. The container closure system for WVE-N531 drug product comprises or consists of a 10 mL USP Type 1 clear glass vial sealed with a FluroTec®-coated rubber stopper and an aluminum overseal with a white flip-off cap. This container closure can protect the sterilized product from microbiological contamination.

A WVE-N531 drug product is typically a sterile solution. In some embodiments, a WVE-N531 drug product can be diluted; in some embodiments, with 0.9% sodium chloride or 0.45% sodium chloride as a sterile, preservative-free solution prior to intravenous (IV) administration. A drug product lot may be made by pooling multiple drug substance lots.

In some embodiments, a drug product has a purity as described herein, e.g., those described for WVE-N531 or a salt thereof (e.g., hexadecasodium salt salt), a WVE-N531 drug substance, etc. In some embodiments, purity is determined assessed using an IP-RP-UPLC method described herein for purity.

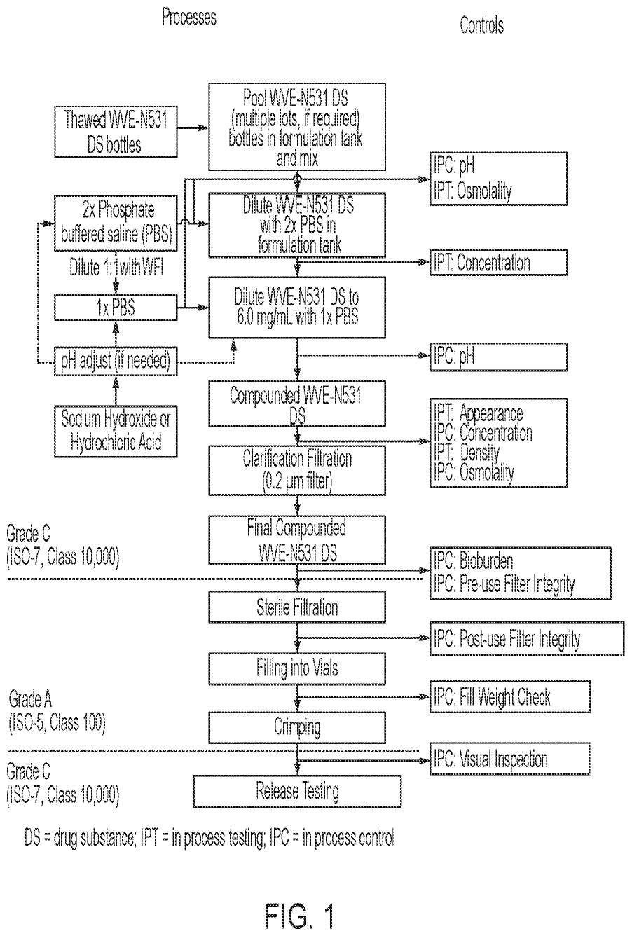

A flow diagram for a WVE-N531 drug product manufacturing process is provided in FIG. 1 as an example.

WVE-N531 drug substance containers can thawed at 2-8° C. for approximately 48 hours. While drug substance thawing occurs, phosphate buffered saline (PBS) solutions at 1× and 2× strengths are prepared where the 1× solution contains 10 mM sodium/potassium phosphate and 142 mM of sodium chloride at pH 7.4±0.2 and 285-315 mOsm/kg osmolality. Contents of the thawed drug substance from each container are pooled into the formulation vessel and mixed adequately.

If a single lot of drug substance is used for formulation, the release purity result is used as the purity correction factor. If multiple lots of drug substance are to be pooled, then a purity correction factor that is based on the quantity of active ingredient from each drug substance lot is determined.

In some embodiments, water for injection (WFI) is used to rinse each drug substance container and the rinsate is added to next drug substance container. The rinsate from final drug substance container is added to the formulation vessel. The total amount of drug substance is weighed and an equal amount of 2×PBS solution is added to the formulation vessel. An in-process sample is taken to measure the concentration by ultraviolet (UV) spectrophotometry and density.

Based on the results of WVE-N531 drug substance concentration and solution mass, the required volume of 1×PBS to accomplish WVE-N531 drug substance concentration of 6.0 mg/mL (WVE-N531 free acid form) is calculated. The required amount of 1×PBS is then added directly to the formulation vessel, and the contents of the vessel are mixed to ensure solution homogeneity.

In some embodiments, final in-process samples are collected from the formulation vessel for concentration by UV spectrophotometry, density, osmolality and pH. In some embodiments, WVE-N531 concentration in a drug product is about 6 mg/mL. In some embodiments, it is about 5.5-6.5 mg/mL (free acid form, purity corrected). The final formulation undergoes clarification filtration.

In some embodiments, an in-process pre-filtration bioburden sample is collected prior to sterile filtration. In some embodiments, the final formulation is sterile filtered through two 0.2 μm filters in series prior to filling. In some embodiments, sterile filters are checked for filter integrity by bubble point method prior to and post filtration.

Based upon the final measured value of density, the target fill weight is determined (to reach target fill volume of 6.35 mL/vial) along with alert limits and action limits. Filling occurs with periodic fill weight checks. The vials are filled, stoppered and sealed aseptically in a Grade A environment. Finished vials are visually inspected prior to release testing, bulk packaging, and/or stability studies.

In some embodiments, process controls and in-process control tests and acceptance limits applied during the manufacture of WVE-N531 drug product include WVE-N531 concentration, pH, osmolality, bioburden, filter integrity, fill weight, appearance, etc.

In some embodiments, manufacture of WVE-N531 drug product comprises in-process concentration measurements via ultraviolet (UV) spectrophotometry. In some embodiments, an in-process concentration measurement is made upon the initial dilution of WVE-N531 drug substance with equal amounts of 2×phosphate buffered saline (PBS). The result is used to determine the amount of 1×PBS to be added for the final concentration which is measured prior to sterile filtration and filling.

Release specifications of WVE-N531 drug product may include one or more specifications described herein, e.g., appearance (e.g., visual), identity (e.g., by retention time of IP-RP-UPLC), purity (e.g., area % by IP-RP-UPLC), impurities (e.g., area % by IP-RP-UPLC), % label claim (e.g., by UV; e.g., +/−10%), pH (e.g., USP <791> and/or Ph. Eur. 2.2.3), osmolality (e.g., USP <785> and/or Ph. Eur. 2.2.35), bacterial endotoxin (e.g., USP <85> and/or Ph. Eur. 2.6.14), sterility (e.g., USP <71> and/or Ph. Eur. 2.6.1), uniformity of dose (e.g., USP <905> and/or Ph. Eur. 2.9.40), particulate matter (e.g., USP <788> and/or Ph. Eur. 2.9.19), container content (e.g., USP <697>), container closure integrity testing (e.g., USP <1207>), etc. In some embodiments, IP-RP-UPLC technologies utilizing one or more or all Set A parameters are utilized for assessing identity (retention time), purity and/or impurities. In some embodiments, purity level is about 70%-85% as described herein. In some embodiments, it is about 70% or more. In some embodiments, it is about 71% or more. In some embodiments, it is about 72% or more. In some embodiments, it is about 73% or more. In some embodiments, it is about 74% or more. In some embodiments, it is about 75% or more. In some embodiments, it is about 76% or more. In some embodiments, it is about 77% or more. In some embodiments, it is about 78% or more. In some embodiments, it is about 79% or more. In some embodiments, it is about 80% or more. In some embodiments, it is about 81% or more. In some embodiments, it is about 82% or more. In some embodiments, it is about 83% or more. In some embodiments, it is about 84% or more. In some embodiments, it is about 85% or more. In some embodiments, it is measured by IP-RP-UPLC area % at 260 nm using Set A parameters.

A WVE-N531 drug product may comprise similar impurities as a WVE-N531 drug substance at levels as described herein.

In some embodiments, a container closure system for WVE-N531 drug product comprises or consists of USP Type 1 glass vials (10 mL capacity, 20 mm opening) with stoppers molded from FluroTec® coated elastomeric formula 4432/50 gray with aluminum overseals, and white flip-off caps.

In some embodiments, a storage condition for WVE-N531 drug product is −20±5° C. In some embodiments, a condition of 5° C.±3° C., e.g., for relatively short term storage.

Among other things, the present disclosure provides the following Example Embodiments:

1. A method for treating muscular dystrophy, comprising administering to a subject suffering therefrom WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the subject has a mutation of the DMD gene that is amenable to exon 53 skipping.

2. A method for treating muscular dystrophy in a subject suffering therefrom, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, and wherein the subject has a mutation in the DMD gene that is amenable to exon 53 skipping.

3. A method, comprising administering to a subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

4. A method, comprising administering to a subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

5. A method for providing DMD exon 53 skipping in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

6. A method for providing DMD exon 53 skipping in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

7. A method for restoring DMD RNA reading frame in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

8. A method for restoring DMD RNA reading frame in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

9. A method for providing a DMD polypeptide in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the DMD polypeptide is truncated compared to a wild-type DMD protein.

10. A method for providing a DMD polypeptide in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the DMD polypeptide is truncated compared to a wild-type DMD protein.

11. The method of any one of Embodiments 9-10, wherein the DMD polypeptide is encoded by an exon 53-skipped DMD mRNA.

12. The method of any one of Embodiments 9-11, wherein the DMD polypeptide provides one or more functions of a wild-type DMD protein.

13. A method for providing increased level of a DMD function in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

14. A method for providing increased level of a DMD function in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

15. The method of any one of Embodiments 3-14, wherein the subject is suffering from a muscular dystrophy.

16. The method of any one of Embodiments 3-15, wherein the subject is suffering from DMD.

17. The method of any one of Embodiments 3-16, wherein the subject has a mutation in the DMD gene that is amenable to exon 53 skipping.

18. The method of any one of the preceding Embodiments, wherein WVE-N531 is administered in one or more forms.

19. The method of any one of the preceding Embodiments, wherein WVE-N531 is administered in one or more pharmaceutically acceptable salt forms.

20. The method of any one of Embodiments 18-19, wherein one form is WVE-N531 hexadecasodium salt.

21. The method of any one of Embodiments 18-20, wherein each dose of WVE-N531 is independently administered in a pharmaceutical composition comprising WVE-N531 and a pharmaceutically acceptable carrier.

22. A method for treating muscular dystrophy, comprising administering to a subject suffering therefrom a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the subject has a mutation of the DMD gene that is amenable to exon 53 skipping.

23. A method, comprising administering to a subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

24. A method for providing DMD exon 53 skipping in a subject, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

25. A method for restoring DMD RNA reading frame, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

26. A method for providing a DMD polypeptide in a subject, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the DMD polypeptide is truncated compared to a wild-type DMD protein.

27. The method of Embodiment 26, wherein the DMD polypeptide is encoded by an exon 53-skipped DMD mRNA.

28. The method of any one of Embodiments 26-27, wherein the DMD polypeptide provides one or more functions of a wild-type DMD protein.

29. A method for providing increased level of a DMD function in a subject, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

30. The method of any one of Embodiments 23-29, wherein the subject is suffering from a muscular dystrophy.

31. The method of any one of Embodiments 23-30, wherein the subject is suffering from DMD.

32. The method of any one of Embodiments 23-31, wherein the subject has a mutation in the DMD gene that is amenable to exon 53 skipping.

33. The method of any one of the preceding Embodiments, wherein WVE-N531 in a dose is equivalent to about 1 mg/kg WVE-N531 free acid form.

34. The method of any one of the preceding Embodiments, wherein WVE-N531 in a dose is equivalent to about 1 mg/kg WVE-N531 free acid form.

35. The method of any one of the preceding Embodiments, wherein WVE-N531 in a dose is equivalent to about 2 mg/kg WVE-N531 free acid form.

36. The method of any one of the preceding Embodiments, wherein WVE-N531 in a dose is equivalent to about 3 mg/kg WVE-N531 free acid form.

37. The method of any one of the preceding Embodiments, wherein WVE-N531 in a dose is equivalent to about 4 mg/kg WVE-N531 free acid form.

38. The method of any one of the preceding Embodiments, wherein WVE-N531 in a dose is equivalent to about 5 mg/kg WVE-N531 free acid form.

39. The method of any one of the preceding Embodiments, wherein WVE-N531 in a dose is equivalent to about 6 mg/kg WVE-N531 free acid form.

40. The method of any one of the preceding Embodiments, wherein WVE-N531 in a dose is equivalent to about 7 mg/kg WVE-N531 free acid form.

41. The method of any one of the preceding Embodiments, wherein WVE-N531 in a dose is equivalent to about 8 mg/kg WVE-N531 free acid form.

42. The method of any one of the preceding Embodiments, wherein WVE-N531 in a dose is equivalent to about 9 mg/kg WVE-N531 free acid form.

43. The method of any one of the preceding Embodiments, wherein WVE-N531 in a dose is equivalent to about 10 mg/kg WVE-N531 free acid form.

44. The method of any one of the preceding Embodiments, wherein WVE-N531 in a dose is equivalent to about 11 mg/kg WVE-N531 free acid form.

45. The method of any one of the preceding Embodiments, wherein WVE-N531 in a dose is equivalent to about 12 mg/kg WVE-N531 free acid form.

46. The method of any one of the preceding Embodiments, wherein WVE-N531 in a dose is equivalent to about 13 mg/kg WVE-N531 free acid form.

47. The method of any one of the preceding Embodiments, wherein WVE-N531 in a dose is equivalent to about 14 mg/kg WVE-N531 free acid form.

48. The method of any one of the preceding Embodiments, wherein WVE-N531 in a dose is equivalent to about 15 mg/kg WVE-N531 free acid form.

49. The method of any one of the preceding Embodiments, wherein WVE-N531 in a dose is equivalent to about 16 mg/kg WVE-N531 free acid form.

50. The method of any one of the preceding Embodiments, wherein WVE-N531 in a dose is equivalent to about 17 mg/kg WVE-N531 free acid form.

51. The method of any one of the preceding Embodiments, wherein WVE-N531 in a dose is equivalent to about 18 mg/kg WVE-N531 free acid form.

52. The method of any one of the preceding Embodiments, wherein WVE-N531 in a dose is equivalent to about 19 mg/kg WVE-N531 free acid form.

53. The method of any one of the preceding Embodiments, wherein WVE-N531 in a dose is equivalent to about 20 mg/kg WVE-N531 free acid form.

54. The method of any one of Embodiments 33-53, wherein about is ±1%.

55. The method of any one of Embodiments 33-53, wherein about is ±2%.

56. The method of any one of Embodiments 33-53, wherein about is ±3%.

57. The method of any one of Embodiments 33-53, wherein about is +4%.

58. The method of any one of Embodiments 33-53, wherein about is ±5%.

59. The method of any one of Embodiments 33-53, wherein about is ±6%.

60. The method of any one of Embodiments 33-53, wherein about is ±7%.

61. The method of any one of Embodiments 33-53, wherein about is ±8%.

62. The method of any one of Embodiments 33-53, wherein about is ±9%.

63. The method of any one of Embodiments 33-53, wherein about is ±10%.

64. The method of any one of the preceding Embodiments, wherein two or more (e.g., about 2, 3, 4, 5, 6, 7, 8, 9, 10, 11, 12, 13, 14, 15, 16, 17, 18, 19, 20, 21, 22, 23, 24, 25, 26, 27, 28, 29 or 30) doses are administered.

65. The method of any one of the preceding Embodiments, wherein about 10 or more doses are administered.

66. The method of any one of Embodiments 64-65, wherein each dose is independently administered in a pharmaceutical composition comprising WVE-N531 and a pharmaceutically acceptable carrier.

67. The method of any one of Embodiments 64-66, wherein each dose has about the same amount of WVE-N531.

68. The method of any one of Embodiments 64-67, wherein WVE-N531 in each dose is equivalent to about 1 mg/kg WVE-N531 free acid form.

69. The method of any one of Embodiments 64-67, wherein WVE-N531 in each dose is equivalent to about 1 mg/kg WVE-N531 free acid form.

70. The method of any one of Embodiments 64-67, wherein WVE-N531 in each dose is equivalent to about 2 mg/kg WVE-N531 free acid form.

71. The method of any one of Embodiments 64-67, wherein WVE-N531 in each dose is equivalent to about 3 mg/kg WVE-N531 free acid form.

72. The method of any one of Embodiments 64-67, wherein WVE-N531 in each dose is equivalent to about 4 mg/kg WVE-N531 free acid form.

73. The method of any one of Embodiments 64-67, wherein WVE-N531 in each dose is equivalent to about 5 mg/kg WVE-N531 free acid form.

74. The method of any one of Embodiments 64-67, wherein WVE-N531 in each dose is equivalent to about 6 mg/kg WVE-N531 free acid form.

75. The method of any one of Embodiments 64-67, wherein WVE-N531 in each dose is equivalent to about 7 mg/kg WVE-N531 free acid form.

76. The method of any one of Embodiments 64-67, wherein WVE-N531 in each dose is equivalent to about 8 mg/kg WVE-N531 free acid form.

77. The method of any one of Embodiments 64-67, wherein WVE-N531 in each dose is equivalent to about 9 mg/kg WVE-N531 free acid form.

78. The method of any one of Embodiments 64-67, wherein WVE-N531 in each dose is equivalent to about 10 mg/kg WVE-N531 free acid form.

79. The method of any one of Embodiments 64-67, wherein WVE-N531 in each dose is equivalent to about 11 mg/kg WVE-N531 free acid form.

80. The method of any one of Embodiments 64-67, wherein WVE-N531 in each dose is equivalent to about 12 mg/kg WVE-N531 free acid form.

81. The method of any one of Embodiments 64-67, wherein WVE-N531 in each dose is equivalent to about 13 mg/kg WVE-N531 free acid form.

82. The method of any one of Embodiments 64-67, wherein WVE-N531 in each dose is equivalent to about 14 mg/kg WVE-N531 free acid form.

83. The method of any one of Embodiments 64-67, wherein WVE-N531 in each dose is equivalent to about 15 mg/kg WVE-N531 free acid form.

84. The method of any one of Embodiments 64-67, wherein WVE-N531 in each dose is equivalent to about 16 mg/kg WVE-N531 free acid form.

85. The method of any one of Embodiments 64-67, wherein WVE-N531 in each dose is equivalent to about 17 mg/kg WVE-N531 free acid form.

86. The method of any one of Embodiments 64-67, wherein WVE-N531 in each dose is equivalent to about 18 mg/kg WVE-N531 free acid form.

87. The method of any one of Embodiments 64-67, wherein WVE-N531 in each dose is equivalent to about 19 mg/kg WVE-N531 free acid form.

88. The method of any one of Embodiments 64-67, wherein WVE-N531 in each dose is equivalent to about 20 mg/kg WVE-N531 free acid form.

89. The method of any one of Embodiments 67-88, wherein about is 1%.

90. The method of any one of Embodiments 67-88, wherein about is ±2%.

91. The method of any one of Embodiments 67-88, wherein about is ±3%.

92. The method of any one of Embodiments 67-88, wherein about is ±4%.

93. The method of any one of Embodiments 67-88, wherein about is +5%.

94. The method of any one of Embodiments 67-88, wherein about is ±6%.

95. The method of any one of Embodiments 67-88, wherein about is ±7%.

96. The method of any one of Embodiments 67-88, wherein about is ±8%.

97. The method of any one of Embodiments 67-88, wherein about is ±9%.

98. The method of any one of Embodiments 67-88, wherein about is ±10%.

99. The method of any one of the preceding Embodiments, wherein WVE-N531 exists in the pharmaceutical composition as one or more pharmaceutically acceptable salt forms.

100. The method of Embodiment 99, wherein a pharmaceutically acceptable salt form is hexadecasodium salt.

101. The method of any one of the preceding Embodiments, wherein a pharmaceutical composition is a liquid composition comprising dissolved WVE-N531.

102. The method of any one of the preceding Embodiments, wherein a pharmaceutically acceptable carrier is or comprises a phosphate buffered solution.

103. The method of any one of the preceding Embodiments, wherein the components in a pharmaceutical composition are WVE-N531, potassium phosphate monobasic, sodium phosphate dibasic, sodium chloride and water, and hydrochloric acid and/or sodium hydroxide for pH adjustment.

104. The method of any one of the preceding Embodiments, wherein a pharmaceutical composition is isotonic.

105. The method of any one of the preceding Embodiments, wherein a pharmaceutical composition has a pH of about 7-8.

106. The method of any one of the preceding Embodiments, wherein a pharmaceutical composition has a pH of about 7.3.

107. The method of Embodiment 105, wherein a pharmaceutical composition has a pH of about 7.4.

108. The method of any one of the preceding Embodiments, wherein two or more consecutive doses are administered about weekly, or about every 2, 3, 4, 5, 6, 7, 8, 9, 10 weeks, or about every 1, 2, 3, 4, 5, 6, or more months.

109. The method of any one of the preceding Embodiments, wherein 10 or more consecutive doses are administered about weekly, or about every 2, 3, 4, 5, 6, 7, 8, 9, 10 weeks, or about every 1, 2, 3, 4, 5, 6, or more months.

110. The method of any one of the preceding Embodiments, wherein all doses are administered about weekly, or about every 2, 3, 4, 5, 6, 7, 8, 9, 10 weeks, or about every 1, 2, 3, 4, 5, 6, or more months.

111. The method of any one of the preceding Embodiments, wherein two or more consecutive doses are administered about every two weeks.

112. The method of any one of the preceding Embodiments, wherein three consecutive doses are administered about every two weeks.

113. The method of any one of the preceding Embodiments, wherein three or more consecutive doses are administered about every two weeks.

114. The method of any one of the preceding Embodiments, wherein 10 or more consecutive doses are administered about every two weeks.

115. The method of any one of the preceding Embodiments, wherein all doses are administered about every two weeks.

116. The method of any one of Embodiments 1-114, wherein two or more consecutive doses are administered about every four weeks.

117. The method of any one of Embodiments 1-114, wherein 10 or more consecutive doses are administered about every four weeks.

118. The method of any one of Embodiments 108-117, wherein each of the consecutive doses is independently equivalent to about 10 mg/kg WVE-N531 free acid form.

119. The method of any one of Embodiments 1-110, wherein all doses are administered about every four weeks.

120. The method of any one of Embodiments 1-110, wherein one or more doses are administered about every two weeks for about 2, 3, 4, 5, 6, 7, 8, 9, 10, 11, 12, 13, 14, 15, or 16 weeks from first dose and then doses are administered about every 3, 4, 5, 6, 7, 8, 9 or 10 weeks.

121. The method of any one of Embodiments 1-110, wherein doses are first administered about every two weeks, and then after about 4 weeks, 8 weeks, 12 weeks, or 16 weeks from first dose, doses are administered about every four weeks.

122. The method of any one of Embodiments 1-110, wherein doses are first administered about every two weeks, and then after about 4 weeks, 8 weeks, 12 weeks, or 16 weeks from first dose, doses are administered about monthly.

123. The method of any one of Embodiments 1-110, wherein two or more (e.g., 2, 3, 4, 5, 6, 7, 8, 9, 10, 11, 12, 13, 14, 15, 16, 17, 18, 19, 20 or more) doses are administered about every two weeks, and two or more (e.g., 2, 3, 4, 5, 6, 7, 8, 9, 10, 11, 12, 13, 14, 15, 16, 17, 18, 19, 20 or more) doses are administered about every four weeks.

124. The method of any one of Embodiments 1-110, wherein two or more (e.g., 2, 3, 4, 5, 6, 7, 8, 9, 10, 11, 12, 13, 14, 15, 16, 17, 18, 19, 20 or more) doses are administered about every two weeks, and two or more (e.g., 2, 3, 4, 5, 6, 7, 8, 9, 10, 11, 12, 13, 14, 15, 16, 17, 18, 19, 20 or more) doses are administered about monthly.

125. The method of any one of Embodiments 123-124, wherein the two or more doses administered about every two weeks are administered before two or more doses administered about every four weeks or monthly.

126. The method of any one of the preceding Embodiments, wherein the composition has a purity of about 70%-85%.

127. The method of any one of the preceding Embodiments, wherein the composition has a purity of about 70%-90%.

128. The method of any one of the preceding Embodiments, wherein the composition has a purity of about 70%-80%.

129. The method of any one of the preceding Embodiments, wherein the composition has a purity of about 70% or more.

130. The method of any one of the preceding Embodiments, wherein the composition has a purity of about 71% or more.

131. The method of any one of the preceding Embodiments, wherein the composition has a purity of about 72% or more.

132. The method of any one of the preceding Embodiments, wherein the composition has a purity of about 73% or more.

133. The method of any one of the preceding Embodiments, wherein the composition has a purity of about 74% or more.

134. The method of any one of the preceding Embodiments, wherein the composition has a purity of about 75% or more.

135. The method of any one of the preceding Embodiments, wherein the composition has a purity of about 76% or more.

136. The method of any one of the preceding Embodiments, wherein the composition has a purity of about 77% or more.

137. The method of any one of the preceding Embodiments, wherein the composition has a purity of about 78% or more.

138. The method of any one of the preceding Embodiments, wherein the composition has a purity of about 79% or more.

139. The method of any one of the preceding Embodiments, wherein the composition has a purity of about 80% or more.

140. The method of any one of the preceding Embodiments, wherein the composition has a purity of about 81% or more.

141. The method of any one of the preceding Embodiments, wherein the composition has a purity of about 82% or more.

142. The method of any one of the preceding Embodiments, wherein the composition has a purity of about 83% or more.

143. The method of any one of the preceding Embodiments, wherein the composition has a purity of about 84% or more.

144. The method of any one of the preceding Embodiments, wherein the composition has a purity of about 85% or more.

145. The method of any one of the preceding Embodiments, wherein the composition has a purity of about 86% or more.

146. The method of any one of the preceding Embodiments, wherein the composition has a purity of about 87% or more.

147. The method of any one of the preceding Embodiments, wherein the composition has a purity of about 88% or more.

148. The method of any one of the preceding Embodiments, wherein the composition has a purity of about 89% or more.

149. The method of any one of the preceding Embodiments, wherein the composition has a purity of about 90% or more.

150. The method of any one of Embodiments 126-149, wherein the purity is measured by IP-RP-UPLC using area % at 260 nm.

151. The method of any one of Embodiments 126-150, wherein the purity is measured by IP-RP-UPLC using area % at 260 nm and the Set A parameters.

152. The method of any one of Embodiments 126-150, wherein the purity is measured by an IP-RP-UPLC method for purity as described herein.

153. The method of any one of the preceding Embodiments, wherein impurities in the composition are no more than about 10%-30%.

154. The method of any one of the preceding Embodiments, wherein impurities in the composition are no more than about 15%-30%.

155. The method of any one of the preceding Embodiments, wherein impurities in the composition are no more than about 20%-30%.

156. The method of any one of the preceding Embodiments, wherein impurities in the composition are no more than about or about 30%.

157. The method of any one of the preceding Embodiments, wherein impurities in the composition are no more than about or about 29%.

158. The method of any one of the preceding Embodiments, wherein impurities in the composition are no more than about or about 28%.

159. The method of any one of the preceding Embodiments, wherein impurities in the composition are no more than about or about 27%.

160. The method of any one of the preceding Embodiments, wherein impurities in the composition are no more than about or about 26%.

161. The method of any one of the preceding Embodiments, wherein impurities in the composition are no more than about or about 25%.

162. The method of any one of the preceding Embodiments, wherein impurities in the composition are no more than about or about 24%.

163. The method of any one of the preceding Embodiments, wherein impurities in the composition are no more than about or about 23%.

164. The method of any one of the preceding Embodiments, wherein impurities in the composition are no more than about or about 22%.

165. The method of any one of the preceding Embodiments, wherein impurities in the composition are no more than about or about 21%.

166. The method of any one of the preceding Embodiments, wherein impurities in the composition are no more than about or about 20%.

167. The method of any one of the preceding Embodiments, wherein impurities in the composition are no more than about or about 19%.

168. The method of any one of the preceding Embodiments, wherein impurities in the composition are no more than about or about 18%.

169. The method of any one of the preceding Embodiments, wherein impurities in the composition are no more than about or about 17%.

170. The method of any one of the preceding Embodiments, wherein impurities in the composition are no more than about or about 16%.

171. The method of any one of the preceding Embodiments, wherein impurities in the composition are no more than about or about 15%.

172. The method of any one of the preceding Embodiments, wherein impurities in the composition are no more than about or about 14%.

173. The method of any one of the preceding Embodiments, wherein impurities in the composition are no more than about or about 13%.

174. The method of any one of the preceding Embodiments, wherein impurities in the composition are no more than about or about 12%.

175. The method of any one of the preceding Embodiments, wherein impurities in the composition are no more than about or about 110%.

176. The method of any one of the preceding Embodiments, wherein impurities in the composition are no more than about or about 10%.

177. The method of any one of Embodiments 153-176, wherein the impurities are measured by IP-RP-UPLC using area % at 260 nm.

178. The method of any one of Embodiments 153-177, wherein the impurities are measured by IP-RP-UPLC using area % at 260 nm and the Set A parameters.

179. The method of any one of Embodiments 153-178, wherein the impurities are measured by an IP-RP-UPLC method for purity as described herein.

180. The method of any one of the preceding Embodiments, wherein stereochemical purity of WVE-N531 is about 80% or more.

181. The method of any one of the preceding Embodiments, wherein stereochemical purity of WVE-N531 is about 85% or more.

182. The method of any one of the preceding Embodiments, wherein stereochemical purity of WVE-N531 is about 80%-90%.

183. The method of any one of the preceding Embodiments, wherein stereochemical purity is assessed by dimer modeling.

184. The method of any one of the preceding Embodiments, wherein the amount of WVE-N531 is measured by UV at 260 nm and 27 OD/mg.

185. The method of any one of the preceding Embodiments, wherein a WVE-N531 drug substance is manufactured by a process described herein.

186. The method of any one of the preceding Embodiments, wherein a WVE-N531 drug substance is characterized by one or more method described herein.

187. The method of any one of the preceding Embodiments, wherein stereochemical identity of WVE-N531 is confirmed by IP-RP-UPLC.

188. The method of any one of the preceding Embodiments, wherein stereochemical identity of WVE-N531 is confirmed by IP-RP-UPLC according to Set B parameters.

189. The method of any one of the preceding Embodiments, wherein stereochemical identity of WVE-N531 is confirmed by an IP-RP-UPLC method for stereochemical identity as described herein.

190. The method of any one of the preceding Embodiments, wherein a WVE-N531 drug substance is released by one or more method described herein.

191. The method of any one of the preceding Embodiments, wherein a WVE-N531 drug substance is stored by one or more method described herein.

192. The method of any one of Embodiments 185-191, wherein the WVE-N531 drug substance is hexadecasodium salt.

193. The method of any one of the preceding Embodiments, wherein a WVE-N531 drug product is manufactured by a process described herein.

194. The method of any one of the preceding Embodiments, wherein a WVE-N531 drug product is characterized by one or more method described herein.

195. The method of any one of the preceding Embodiments, wherein a WVE-N531 drug product is released by one or more method described herein.

196. The method of any one of the preceding Embodiments, wherein a WVE-N531 drug product is stored by one or more method described herein.

197. The method of any one of the preceding Embodiments, wherein a pharmaceutical composition is manufactured by a process described herein.

198. The method of any one of the preceding Embodiments, wherein a pharmaceutical composition is characterized by one or more method described herein.

199. The method of any one of the preceding Embodiments, wherein a pharmaceutical composition is released by one or more method described herein.

200. The method of any one of the preceding Embodiments, wherein a pharmaceutical composition is stored by one or more method described herein.

201. The method of any one of the preceding Embodiments, wherein WVE-N531 is administered intravenously.

202. The method of any one of the preceding Embodiments, wherein a DMD mutation is Δ3-52, Δ4-52, Δ5-52, Δ6-52, Δ9-52, Δ10-52, Δ11-52, Δ13-52, Δ14-52, Δ15-52, Δ16-52, Δ17-52, Δ19-52, Δ21-52, Δ23-52, Δ24-52, Δ25-52, Δ26-52, Δ27-52, Δ28-52, Δ29-52, Δ30-52, Δ31-52, Δ32-52, Δ33-52, Δ34-52, Δ35-52, Δ36-52, Δ37-52, Δ38-52, Δ39-52, Δ40-52, Δ41-52, Δ42-52, Δ43-52, Δ45-52, Δ47-52, Δ48-52, Δ49-52, Δ50-52, Δ51-52, Δ52, Δ54-58, Δ54-61, Δ54-63, Δ54-64, Δ54-66, Δ54-76, or Δ54-77.

203. The method of any one of the preceding Embodiments, wherein the mutation comprises Δ52.

204. The method of any one of the preceding Embodiments, wherein the mutation is Δ52.

205. The method of Embodiment 203, wherein the mutation is Δ45-52.

206. The method of Embodiment 203, wherein the mutation is Δ47-52.

207. The method of Embodiment 203, wherein the mutation is Δ48-52.

208. The method of Embodiment 203, wherein the mutation is Δ49-52.

209. The method of Embodiment 203, wherein the mutation is Δ50-52.

210. The method of Embodiment 203, wherein the mutation is Δ51-52.

211. The method of any one of the preceding Embodiments, wherein the muscular dystrophin is DMD.

212. The method of any one of the preceding Embodiments, wherein exon 53 is skipped in DMD mRNA.

213. The method of any one of the preceding Embodiments, wherein level of exon 53-skipped DMD mRNA is increased.

214. The method of any one of the preceding Embodiments, wherein about 10%, 15%, 20%, 25%, 30%, 35%, 40%, 45%, 50%, 55%, 60%, 65%, 70% or more of DMD mRNA is exon 53-skipped DMD mRNA after administration for a certain time period or after a certain number of doses.

215. The method of any one of the preceding Embodiments, wherein a truncated DMD polypeptide is produced compared to a wild-type DMD protein.

216. The method of any one of the preceding Embodiments, wherein level of a truncated DMD polypeptide is increased.

217. The method of any one of Embodiments 215-216, wherein the truncated DMD polypeptide performs one or more functions of a wild-type DMD protein.

218. The method of any one of the preceding Embodiments, wherein the method provide increases from baseline in dystrophin levels of about 1% or more of normal levels after administration for a certain time period or after a certain number of doses.

219. The method of Embodiment 218, wherein the increases from baseline in dystrophin levels is of about 2% or more of normal levels.

220. The method of Embodiment 218, wherein the increases from baseline in dystrophin levels is of about 5% or more of normal levels.

221. The method of Embodiment 218, wherein the increases from baseline in dystrophin levels is of about 5.3% or more of normal levels.

222. The method of Embodiment 218, wherein the increases from baseline in dystrophin levels is of about 6% or more of normal levels.

223. The method of Embodiment 218, wherein the increases from baseline in dystrophin levels is of about 7% or more of normal levels.

224. The method of Embodiment 218, wherein the increases from baseline in dystrophin levels is of about 8% or more of normal levels.

225. The method of Embodiment 218, wherein the increases from baseline in dystrophin levels is of about 9% or more of normal levels.

226. The method of Embodiment 218, wherein the increases from baseline in dystrophin levels is of about 10% or more of normal levels.

227. The method of Embodiment 218, wherein the increases from baseline in dystrophin levels is of about 11% or more of normal levels.

228. The method of Embodiment 218, wherein the increases from baseline in dystrophin levels is of about 12% or more of normal levels.

229. The method of Embodiment 218, wherein the increases from baseline in dystrophin levels is of about 13% or more of normal levels.

230. The method of Embodiment 218, wherein the increases from baseline in dystrophin levels is of about 14% or more of normal levels.

231. The method of Embodiment 218, wherein the increases from baseline in dystrophin levels is of about 15% or more of normal levels.

232. The method of Embodiment 218, wherein the increases from baseline in dystrophin levels is of about 16% or more of normal levels.

233. The method of Embodiment 218, wherein the increases from baseline in dystrophin levels is of about 17% or more of normal levels.

234. The method of Embodiment 218, wherein the increases from baseline in dystrophin levels is of about 18% or more of normal levels.

235. The method of Embodiment 218, wherein the increases from baseline in dystrophin levels is of about 19% or more of normal levels.

236. The method of Embodiment 218, wherein the increases from baseline in dystrophin levels is of about 20% or more of normal levels.

237. The method of any one of Embodiments 218-236, wherein the increase is measured after administration for about 12 weeks from first dose.

238. The method of any one of Embodiments 218-236, wherein the increase is measured after administration for about 13 weeks from first dose.

239. The method of any one of Embodiments 218-236, wherein the increase is measured after administration for about 14 weeks from first dose.

240. The method of any one of Embodiments 218-236, wherein the increase is measured after administration for about 24 weeks from first dose.

241. The method of any one of Embodiments 218-236, wherein the increase is measured after administration for about 25 weeks from first dose.

242. The method of any one of Embodiments 218-236, wherein the increase is measured after administration for about 26 weeks from first dose.

243. The method of any one of Embodiments 218-236, wherein the increase is measured after administration for about 36 weeks from first dose.

244. The method of any one of Embodiments 218-236, wherein the increase is measured after administration for about 37 weeks from first dose.

245. The method of any one of Embodiments 218-236, wherein the increase is measured after administration for about 38 weeks from first dose.

246. The method of any one of Embodiments 218-236, wherein the increase is measured after administration for about 48 weeks from first dose.

247. The method of any one of Embodiments 218-236, wherein the increase is measured after administration for about 49 weeks from first dose.

248. The method of any one of Embodiments 218-236, wherein the increase is measured after administration for about 50 weeks from first dose.

249. The method of any one of Embodiments 218-236, wherein the increase is measured after administration for about 72 weeks from first dose.

250. The method of any one of Embodiments 218-236, wherein the increase is measured after administration for about 73 weeks from first dose.

251. The method of any one of Embodiments 218-236, wherein the increase is measured after administration for about 74 weeks from first dose.

252. The method of any one of Embodiments 218-236, wherein the increase is measured after administration for about 96 weeks from first dose.

253. The method of any one of Embodiments 218-236, wherein the increase is measured after administration for about 97 weeks from first dose.

254. The method of any one of Embodiments 218-236, wherein the increase is measured after administration for about 98 weeks from first dose.

255. The method of any one of Embodiments 218-254, wherein the increase is measured after 6 doses.

256. The method of any one of Embodiments 218-254, wherein the increase is measured after 7 doses.

257. The method of any one of Embodiments 218-254, wherein the increase is measured after 8 doses.

258. The method of any one of Embodiments 218-254, wherein the increase is measured after 12 doses.

259. The method of any one of Embodiments 218-254, wherein the increase is measured after 13 doses.

260. The method of any one of Embodiments 218-254, wherein the increase is measured after 14 doses.

261. The method of any one of Embodiments 218-254, wherein the increase is measured after 24 doses.

262. The method of any one of Embodiments 218-254, wherein the increase is measured after 25 doses.

263. The method of any one of Embodiments 218-254, wherein the increase is measured after 30 doses.

264. The method of any one of Embodiments 218-254, wherein the increase is measured after 35 doses.

265. The method of any one of Embodiments 218-254, wherein the increase is measured after 40 doses.

266. The method of any one of Embodiments 218-254, wherein the increase is measured after 45 doses.

267. The method of any one of Embodiments 218-254, wherein the increase is measured after 48 doses.

268. The method of any one of the preceding Embodiments, wherein a DMD function is increased.

269. The method of any one of the preceding Embodiments, wherein a DMD function is restored.

270. The method of any one of the preceding Embodiments, wherein loss of ambulation in a subject is reduced.

271. The method of any one of the preceding Embodiments, wherein disease progression in the subject is delayed or slowed.

272. The method of any one of the preceding Embodiments, wherein muscle weakness in the subject is delayed or slowed.

273. The method of any one of the preceding Embodiments, wherein loss of muscle mass in the subject is delayed or slowed.

274. The method of any one of the preceding Embodiments, wherein loss of pulmonary function in the subject is delayed or slowed.

275. The method of any one of the preceding Embodiments, wherein the subject improves in a muscular dystrophy assessment.

276. The method of any one of the preceding Embodiments, wherein the subject improves in a 10 meter walk test.

277. The method of any one of the preceding Embodiments, wherein the subject improves in one or more functional assessments.

278. The method of any one of the preceding Embodiments, wherein the subject improves in North Star Ambulatory Assessment (NSAA) 2.0.

279. The method of any one of the preceding Embodiments, wherein the subject improves in Performance of the Upper Limb (PUL) 2.0.

280. The method of any one of the preceding Embodiments, wherein the subject improves in one or more lower limb motor function by timed function tests.

281. The method of any one of the preceding Embodiments, wherein the subject improves in four-stair climb.

282. The method of any one of the preceding Embodiments, wherein the subject improves in time to rise from the floor.

283. The method of any one of the preceding Embodiments, wherein the subject improves in upper limb proximal strength.

284. The method of any one of the preceding Embodiments, wherein the subject improves in handheld myometry.

285. The method of any one of the preceding Embodiments, wherein the subject improves in one or more pulmonary function tests.

286. The method of any one of the preceding Embodiments, wherein the subject improves in one or more of peak flow rate [PFR], cough peak flow [CPF], and FVC.

287. The method of any one of the preceding Embodiments, wherein an improvement is compared to baseline.

288. The method of any one of the preceding Embodiments, wherein an improvement is compared to absence of WVE-N531 administration.

289. The method of any one of the preceding Embodiments, wherein an improvement is compared to administration of a reference composition.

290. The method of any one of the preceding Embodiments, wherein a reference composition is comparable to an administered WVE-N531 composition but does not contain WVE-N531.

291. The method of any of the previous Embodiments, wherein the subject is a pediatric subject.

292. The method of any of the previous Embodiments, wherein the subject is administered a steroid at least about one month prior to the first dose of WVE-N531.

293. The method of any of the previous Embodiments, wherein the subject is administered a steroid at least about six months prior to the first dose of WVE-N531.

294. The method of any of the previous Embodiments, wherein the subject is administered a corticosteroid at least about one month prior to the first dose of WVE-N531.

295. The method of any of the previous Embodiments, wherein the subject is administered a corticosteroid at least about six months prior to the first dose of WVE-N531.

296. The method of any one of Embodiments 294-295, wherein the corticosteroid is deflazacort.

297. A composition comprising WVE-N531.

298. The composition of Embodiment 297, wherein a form of WVE-N531 in the composition is a pharmaceutically acceptable salt form.

299. The composition of any one of Embodiments 297-298, wherein each form of WVE-N531 in the composition is independently a salt form.

300. The composition of any one of Embodiments 297-299, wherein each form of WVE-N531 in the composition is independently a pharmaceutically acceptable salt form.

301. The composition of any one of Embodiments 297-300, wherein a form of WVE-N531 in the composition is WVE-N531 hexadecasodium salt.

302. The composition of any one of the preceding Embodiments, wherein the composition is a drug substance.

303. The composition of any one of Embodiments 297-301, wherein the composition is a drug product.

304. The compound of any one of the preceding Embodiments, wherein the composition is a liquid composition wherein WVE-N531 is dissolved.

305. The composition of any one of the preceding Embodiments, wherein the composition is a pharmaceutical composition further comprising a pharmaceutically acceptable carrier.

306. The composition of Embodiment 305, wherein the pharmaceutically acceptable carrier is or comprises a phosphate buffered solution.

307. The composition of any one of the preceding Embodiments, wherein the components in the composition are WVE-N531, potassium phosphate monobasic, sodium phosphate dibasic, sodium chloride and water, and hydrochloric acid and/or sodium hydroxide for pH adjustment.

308. The composition of any one of the preceding Embodiments, wherein the composition is isotonic.

309. The composition of any one of the preceding Embodiments, wherein the composition has a pH of about 7-8.

310. The composition of any one of the preceding Embodiments, wherein the composition has a pH of about 7.3.

311. The composition of Embodiment 309, wherein a pharmaceutical composition has a pH of about 7.4.

312. The composition of any one of the preceding Embodiments, wherein the concentration of WVE-N531 is equivalent to about 5-45 mg/mL WVE-N531 free acid form.

313. The composition of Embodiment 312, wherein the concentration of WVE-N531 is equivalent to about 25-40 mg/mL WVE-N531 free acid form.

314. The composition of Embodiment 312, wherein the concentration of WVE-N531 is equivalent to about 5-7 mg/mL WVE-N531 free acid form.

315. The composition of Embodiment 312, wherein the concentration of WVE-N531 is equivalent to about 6 mg/mL WVE-N531 free acid form.

316. The composition of Embodiment 315, wherein the composition is packaged into a vial, wherein the volume of the composition in the vial is at least 6 mL.

317. The composition of any one of Embodiments 315-316, wherein the composition is packaged into a vial, wherein the volume of the composition in the vial is at least 6-7 mL.

318. The composition of any one of Embodiments 315-316, wherein the composition is packaged into a vial, wherein the volume of the composition in the vial is at least 6-6.5 mL.

319. The composition of any one of the preceding Embodiments, wherein the composition has a purity of about 70%-90%.

320. The composition of any one of the preceding Embodiments, wherein the composition has a purity of about 70%-85%.

321. The composition of any one of the preceding Embodiments, wherein the composition has a purity of about 70%-80%.

322. The composition of any one of the preceding Embodiments, wherein the composition has a purity of about 70% or more.

323. The composition of any one of the preceding Embodiments, wherein the composition has a purity of about 71% or more.

324. The composition of any one of the preceding Embodiments, wherein the composition has a purity of about 72% or more.

325. The composition of any one of the preceding Embodiments, wherein the composition has a purity of about 73% or more.

326. The composition of any one of the preceding Embodiments, wherein the composition has a purity of about 74% or more.

327. The composition of any one of the preceding Embodiments, wherein the composition has a purity of about 75% or more.

328. The composition of any one of the preceding Embodiments, wherein the composition has a purity of about 76% or more.

329. The composition of any one of the preceding Embodiments, wherein the composition has a purity of about 77% or more.

330. The composition of any one of the preceding Embodiments, wherein the composition has a purity of about 78% or more.

331. The composition of any one of the preceding Embodiments, wherein the composition has a purity of about 79% or more.

332. The composition of any one of the preceding Embodiments, wherein the composition has a purity of about 80% or more.

333. The composition of any one of the preceding Embodiments, wherein the composition has a purity of about 81% or more.

334. The composition of any one of the preceding Embodiments, wherein the composition has a purity of about 82% or more.

335. The composition of any one of the preceding Embodiments, wherein the composition has a purity of about 83% or more.

336. The composition of any one of the preceding Embodiments, wherein the composition has a purity of about 84% or more.

337. The composition of any one of the preceding Embodiments, wherein the composition has a purity of about 85% or more.

338. The composition of any one of the preceding Embodiments, wherein the composition has a purity of about 86% or more.

339. The composition of any one of the preceding Embodiments, wherein the composition has a purity of about 87% or more.

340. The composition of any one of the preceding Embodiments, wherein the composition has a purity of about 88% or more.

341. The composition of any one of the preceding Embodiments, wherein the composition has a purity of about 89% or more.

342. The composition of any one of the preceding Embodiments, wherein the composition has a purity of about 90% or more.

343. The composition of any one of Embodiments 319-342, wherein the purity is measured by IP-RP-UPLC using area % at 260 nm.

344. The composition of any one of Embodiments 319-342, wherein the purity is measured by IP-RP-UPLC using area % at 260 nm and the Set A parameters.

345. The composition of any one of Embodiments 319-342, wherein the purity is measured by an IP-RP-UPLC method for purity as described herein.

346. The composition of any one of the preceding Embodiments, wherein impurities in the composition are no more than about 10%-30%.

347. The composition of any one of the preceding Embodiments, wherein impurities in the composition are no more than about 15%-30%.

348. The composition of any one of the preceding Embodiments, wherein impurities in the composition are no more than about 20%-30%.

349. The composition of any one of the preceding Embodiments, wherein impurities in the composition are no more than about or about 30%.

350. The composition of any one of the preceding Embodiments, wherein impurities in the composition are no more than about or about 29%.

351. The composition of any one of the preceding Embodiments, wherein impurities in the composition are no more than about or about 28%.

352. The composition of any one of the preceding Embodiments, wherein impurities in the composition are no more than about or about 27%.

353. The composition of any one of the preceding Embodiments, wherein impurities in the composition are no more than about or about 26%.

354. The composition of any one of the preceding Embodiments, wherein impurities in the composition are no more than about or about 25%.

355. The composition of any one of the preceding Embodiments, wherein impurities in the composition are no more than about or about 24%.

356. The composition of any one of the preceding Embodiments, wherein impurities in the composition are no more than about or about 23%.

357. The composition of any one of the preceding Embodiments, wherein impurities in the composition are no more than about or about 22%.

358. The composition of any one of the preceding Embodiments, wherein impurities in the composition are no more than about or about 210%.

359. The composition of any one of the preceding Embodiments, wherein impurities in the composition are no more than about or about 20%.

360. The composition of any one of the preceding Embodiments, wherein impurities in the composition are no more than about or about 19%.

361. The composition of any one of the preceding Embodiments, wherein impurities in the composition are no more than about or about 18%.

362. The composition of any one of the preceding Embodiments, wherein impurities in the composition are no more than about or about 17%.

363. The composition of any one of the preceding Embodiments, wherein impurities in the composition are no more than about or about 16%.

364. The composition of any one of the preceding Embodiments, wherein impurities in the composition are no more than about or about 15%.

365. The composition of any one of the preceding Embodiments, wherein impurities in the composition are no more than about or about 14%.

366. The composition of any one of the preceding Embodiments, wherein impurities in the composition are no more than about or about 13%.

367. The composition of any one of the preceding Embodiments, wherein impurities in the composition are no more than about or about 12%.

368. The composition of any one of the preceding Embodiments, wherein impurities in the composition are no more than about or about 11%.

369. The composition of any one of the preceding Embodiments, wherein impurities in the composition are no more than about or about 10%.

370. The composition of any one of Embodiments 346-369, wherein the impurities are measured by IP-RP-UPLC using area % at 260 nm.

371. The composition of any one of Embodiments 346-370, wherein the impurities are measured by IP-RP-UPLC using area % at 260 nm and the Set A parameters.

372. The composition of any one of Embodiments 346-371, wherein the impurities are measured by an IP-RP-UPLC method for purity as described herein.

373. The composition of any one of the preceding Embodiments, wherein stereochemical purity of WVE-N531 is about 80% or more.

374. The composition of any one of the preceding Embodiments, wherein stereochemical purity of WVE-N531 is about 85% or more.

375. The composition of any one of the preceding Embodiments, wherein stereochemical purity of WVE-N531 is about 80%-90%.

376. The composition of any one of the preceding Embodiments, wherein stereochemical purity is assessed by dimer modeling.

377. The composition of any one of the preceding Embodiments, wherein the amount of WVE-N531 is measured by UV at 260 nm.

378. The composition of any one of the preceding Embodiments, wherein the amount of WVE-N531 is measured by UV at 260 nm and 27 OD/mg.

379. The composition of any one of the preceding Embodiments, wherein the WVE-N531 drug substance in the composition is manufactured by a process described herein.

380. The composition of any one of the preceding Embodiments, wherein the WVE-N531 drug substance in the composition is characterized by one or more method described herein.

381. The composition of any one of the preceding Embodiments, wherein the WVE-N531 drug substance in the composition is released by one or more method described herein.

382. The composition of any one of the preceding Embodiments, wherein the WVE-N531 drug substance is stored by one or more method described herein.

383. The composition of any one of Embodiments 379-382, wherein the WVE-N531 drug substance is hexadecasodium salt.

384. The composition of any one of the preceding Embodiments, wherein the composition is a WVE-N531 drug product.

385. The composition of any one of the preceding Embodiments, wherein WVE-N531 drug product is manufactured by a process described herein.

386. The composition of any one of the preceding Embodiments, wherein WVE-N531 drug product is characterized by one or more method described herein.

387. The composition of any one of the preceding Embodiments, wherein WVE-N531 drug product is released by one or more method described herein.

388. The composition of any one of the preceding Embodiments, wherein WVE-N531 drug product is stored by one or more method described herein.

389. The composition of any one of the preceding Embodiments, wherein the composition is manufactured by a process described herein.

390. The composition of any one of the preceding Embodiments, wherein a pharmaceutical composition is characterized by one or more method described herein.

391. The composition of any one of the preceding Embodiments, wherein a pharmaceutical composition is released by one or more method described herein.

392. The composition of any one of the preceding Embodiments, wherein a pharmaceutical composition is stored by one or more method described herein.

393. The composition of any one of the preceding Embodiments, wherein the composition does not contain DS1.

394. The composition of any one of the preceding Embodiments, wherein the composition does not contain DS2.

395. The composition of any one of the preceding Embodiments, wherein the composition does not contain DS3.

396. The composition of any one of the preceding Embodiments, wherein the composition does not contain DS4.

397. The composition of any one of the preceding Embodiments, wherein the composition does not contain DS5.

398. The composition of any one of the preceding Embodiments, wherein the composition does not contain DS6.

399. The composition of any one of the preceding Embodiments, wherein the composition does not contain DS7.

400. The composition of any one of the preceding Embodiments, wherein the composition does not contain DS8.

401. The composition of any one of the preceding Embodiments, wherein the composition does not contain DS9.

402. The composition of any one of the preceding Embodiments, wherein the composition does not contain DS10.

403. The composition of any one of the preceding Embodiments, wherein the composition does not contain DS11.

404. The composition of any one of the preceding Embodiments, wherein the composition does not contain DS12.

405. The composition of any one of the preceding Embodiments, wherein the composition does not contain DS13.

406. The composition of any one of the preceding Embodiments, wherein the composition does not contain DS14.

407. The composition of any one of the preceding Embodiments, wherein the composition does not contain DS15.

408. The composition of any one of the preceding Embodiments, wherein the composition does not contain DS16.

409. The composition of any one of the preceding Embodiments, wherein the composition does not contain DS17.

410. A composition comprising one of WVE-N531 and DS1 to DS17, wherein the composition is free of one or more (e.g., 1, 2, 3, 4, 5, 6, 7, 8, 9, 10, 11, 12, 13, 14, 15, 16 or 17) of the rest of WVE-N531 and DS1 to DS17.

411. The composition of Embodiment 410, wherein the composition comprises WVE-N531.

412. The composition of Embodiment 410, wherein the composition comprises DS1.

413. The composition of Embodiment 410, wherein the composition comprises DS2.

414. The composition of Embodiment 410, wherein the composition comprises DS3.

415. The composition of Embodiment 410, wherein the composition comprises DS4.

416. The composition of Embodiment 410, wherein the composition comprises DS5.

417. The composition of Embodiment 410, wherein the composition comprises DS6.

418. The composition of Embodiment 410, wherein the composition comprises DS7.

419. The composition of Embodiment 410, wherein the composition comprises DS8.

420. The composition of Embodiment 410, wherein the composition comprises DS9.

421. The composition of Embodiment 410, wherein the composition comprises DS10.

422. The composition of Embodiment 410, wherein the composition comprises DS11.

423. The composition of Embodiment 410, wherein the composition comprises DS12.

424. The composition of Embodiment 410, wherein the composition comprises DS13.

425. The composition of Embodiment 410, wherein the composition comprises DS14.

426. The composition of Embodiment 410, wherein the composition comprises DS15.

427. The composition of Embodiment 410, wherein the composition comprises DS16.

428. The composition of Embodiment 410, wherein the composition comprises DS17.

429. The composition of any one of the preceding Embodiments, wherein about is ±10%.

430. The composition of any one of the preceding Embodiments, wherein about is ±2%.

431. The composition of any one of the preceding Embodiments, wherein about is ±3%.

432. The composition of any one of the preceding Embodiments, wherein about is ±4%.

433. The composition of any one of the preceding Embodiments, wherein about is ±5%.

434. The composition of any one of the preceding Embodiments, wherein about is ±6%.

435. The composition of any one of the preceding Embodiments, wherein about is ±7%.

436. The composition of any one of the preceding Embodiments, wherein about is ±8%.

437. The composition of any one of the preceding Embodiments, wherein about is ±9%.

438. The composition of any one of the preceding Embodiments, wherein about is ±10%.

439. A method for manufacturing a WVE-N531 composition according to a method described in the specification.

440. The method of Embodiment 439, comprising utilizing IP-RP-UPLC to assess purity and/or impurities in the manufactured WVE-N531 composition and release the preparation if the purity and/or impurities meet certain criteria.

441. The method of any one of Embodiments 439-440, wherein the composition is a drug substance.

442. The method of any one of Embodiments 439-440, wherein the composition is a drug product.

443. A method for releasing a WVE-N531 preparation, comprising utilizing IP-RP-UPLC to assess purity and/or impurities in the WVE-N531 preparation and release the preparation if the purity and/or impurities meet certain criteria.

444. A method for assessing purity of WVE-N531 utilizing IP-RP-UPLC.

445. The method of any one of Embodiment 440-444, wherein the IP-RP-UPLC utilized one or more parameters described in the specification.

446. The method of any one of Embodiment 440-444, wherein the IP-RP-UPLC utilized one or more parameters of Set A.

447. The method of any one of Embodiment 440-444, wherein the IP-RP-UPLC utilized the Set A parameters.

448. The method of any one of Embodiments 439-447, wherein stereochemical identity of WVE-N531 is confirmed by IP-RP-UPLC.

449. A method for confirming stereochemical identity of WVE-N531 utilizing IP-RP-UPLC.

450. The method of any one of Embodiments 439-449, wherein stereochemical identity of WVE-N531 is confirmed by IP-RP-UPLC according to Set B parameters.

451. The method of any one of Embodiments 439-449, wherein stereochemical identity of WVE-N531 is confirmed by an IP-RP-UPLC method for stereochemical identity as described herein.

452. The method of any one of Embodiments 439-451, wherein the composition is any one of the preceding Embodiments.

453. A compound, oligonucleotide, composition, method, process, use, dose or dosage regimen described in the specification.

### EXEMPLIFICATION

The foregoing has been a description of certain non-limiting embodiments of the disclosure. Accordingly, it is to be understood that embodiments of the disclosure herein described are merely illustrative of applications of principles of the disclosure. Reference herein to details of illustrated embodiments is not intended to limit the scope of any claims.

Functions and advantage of certain embodiments of the present disclosure may be more fully understood from the examples described below. The following examples are intended to illustrate certain benefits of such embodiments.

### Example 1. WVE-N531 are Sufficiently Safe for Human Administration Based on Studies In Vitro and in Animals

Among other things, the present disclosure provides data including those in primates demonstrating that WVE-N531 can be administered to human subjects according to various doses and dosage regimens as described herein.

WVE-N531 is an oligonucleotide that can target human Duchenne muscular dystrophy (DMD) messenger ribonucleic acid (mRNA) to induce exon 53 skipping and restoration of functional dystrophin protein in patients with DMD.

Among other things, WVE-N531 induced exon skipping and protein restoration in patient-derived cell lines. In vivo, exon skipping, dystrophin protein restoration, and improvements in survival were achieved in the mdx23 and/or double knockout (dKO) mouse models using a surrogate molecule. These models have a point mutation in exon 23, and therefore a murine surrogate oligonucleotide with a sequence intended to specifically target mouse exon 23 with a phosphoramidate (PN) backbone that mimics the chemistry of WVE-N531 was used in these experiments. In vivo target engagement was also demonstrated in healthy cynomolgus monkeys, a pharmacologically relevant species, using WVE-N531. In dystrophin-deficient muscle, biodistribution and exon skipping may be greater than that in healthy, normal muscle, which has been observed with other oligonucleotides.

WVE-N531 was assessed in nonclinical toxicity studies, and no risks have been identified that would prevent dosing of WVE-N531 in humans. Following 14-week repeat dosing in monkeys at 45 mg/kg, effects were considered non-adverse due to the mild severity of findings, infrequent incidence, and/or the lack of impact on the health and well-being of the animals. The no-observed-adverse-effect level (NOAEL) was considered to be at least 45 mg/kg/dose, the highest dose evaluated.

Other findings observed in both mice and monkeys were consistent with those commonly observed with oligonucleotides, included non-adverse reversible to partially reversible renal and hepatic findings, pro-inflammatory effects, and transient effects on coagulation factors [prothrombin time (PT), activated partial thromboplastin time (aPTT), and fibrinogen]. These risks are easily monitorable in the clinic.

It was observed that WVE-N531 was not associated with in vitro or in vivo genotoxicity or off-target effects via hybridization to the human genome. In vitro, WVE-N531 did not activate Toll-like receptor 9 (TLR9) in human reporter cells or increase pro-inflammatory cytokines in human peripheral blood mononuclear cells (PBMC) ex vivo.

WVE-N531 did not affect cardiovascular or respiratory function in monkeys up to the highest dose evaluated (45 mg/kg). No adverse effects on CNS function were observed in mice up to the highest dose evaluated (180 mg/kg).

Various findings observed in various nonclinical studies are consistent with class effects observed with other oligonucleotides.

WVE-N531 has been demonstrated to provide DMD exon skipping and to restore dystrophin protein in DMD patient-derived myoblast cell lines (e.g., DMDΔ45-52 and DMDΔ52). Further, a mouse exon 23 specific oligonucleotide with comparable chemistry to WVE-N531, WV-21218, has demonstrated a dose response effect on exon 23 skipping and dystrophin restoration in the mdx23 mouse model of DMD. This oligonucleotide also demonstrated exon skipping, dystrophin protein restoration and other biological effects including extended life span in a dKO mouse model for DMD, which lacks both utrophin and dystrophin and develops a severe muscular dystrophy phenotype comparable to that observed in patients with DMD. See, e.g., WO 2021/237223.

Bioinformatics tools were used to determine potential off-target effects based on sequence homology to WVE-N531. The analysis suggested that sequence dependent off-target effects is minimal.

WVE-N531 was analyzed for the ability to activate the innate immune protein TLR9 in a cell-based reporter assay. WVE-N531 exposure to tested cells (human embryonic kidney cells overexpressing human TLR9) did not increase TLR9 reporter activity at concentrations up to 30 M compared to water-treated cells (negative control). The potential of WVE-N531 to activate human TLR9 is negligible based on these test data.

Immune-activation potential of WVE-N531 was also evaluated by incubating WVE-N531 with PBMC from healthy human donors followed by assessment of cytokine release. Cytokine responses to WVE-N531 were not statistically-significantly different from cytokine responses to water, for any of 7 analytes: IFNα2, IL12p40, IL-1β, IL-6, MIP-1α, MIP-1β, TNFα. A statistically significant decrease in MCP-1 levels was observed compared to both the negative (H2O) and positive control (TLR7/TLR8 agonist R848). These data showed WVE-N531 has negligible immune stimulatory potential as measured in this ex vivo PBMC assay.

The effects of WVE-N531 on cardiovascular and respiratory function in cynomolgus monkeys and CNS function in CD-1 mice following IV administration were evaluated in GLP studies. The study designs overview is presented in the Table below (IV=intravenous):

| | | | | GLP |
|---|---|---|---|---|
| Species and | Method of | Duration of | | Compli- |
| Strain | Admin | Dosing | Dose (mg/kg) | ance |
| CD-1 Mouse | IV, bolus | Repeat dose | 0, 15, 60, 180 | Yes |
| Cynomolgus | IV, 60-minute | Single dose | 0, 3, 15, 45 | Yes |
| Monkcy | infusion | | | |
| Cynomolgus | IV, 60-minute | Repeat dose | 0, 3, 15, 45 | Yes |
| Monkey | infusion | | | |

In a study in mice, the effects of WVE-N531 on CNS function were assessed in male and female CD-1 mice following vehicle (dPBS without calcium or magnesium) or 15, 60, or 180 mg/kg/dose by IV (bolus) injection of WVE-N531 every other week on Days 1, 15, 29, 43, 57, 71, 85, and 99. According to the functional observational battery (FOB) data, no adverse CNS effects were observed at doses up to 180 mg/kg/dose.

In one study in monkeys, the effects of WVE-N531 on cardiovascular and respiratory function was evaluated in conscious radiotelemetry-instrumented male cynomolgus monkeys after a single dose of WVE-N531. Animals received WVE-N531 (3, 15, or 45 mg/kg) or dPBS (without calcium or magnesium) via 60-minute IV infusion. No WVE-N531-related effects on cardiovascular or respiratory function were observed following administration of up to 45 mg/kg of WVE-N531. Dose-dependent increases in complement Factor Bb and C3a plasma concentrations were observed at 2 min post EOI at 15 and 45 mg/kg WVE-N531. Compared to control, increases in Factor Bb were 3.7-fold and 34.6-fold at dose levels of 15 and 45 mg/kg, respectively; increases in C3a were 2.0-fold and 32.4-fold at dose levels of 15 and 45 mg/kg, respectively at 2 minutes post EOI, with a return to baseline by 24 hours.

In another study in monkeys, the effects of WVE-N531 on cardiovascular function was evaluated in anesthetized male and female cynomolgus monkeys after repeated administration of WVE-N531. Animals received vehicle (dPBS without calcium or magnesium) or 3, 15 or 45 mg/kg doses of WVE-N531 via 1-hour IV infusions, every other week. Electrocardiograms (ECGs) were collected once during the predose phase and 0.5 hours (+0.25 hours) postdose on Days 15 and 71 of the dosing phase. No effects were observed following 1-hour IV infusion administration of WVE-N531 to male or female cynomolgus monkeys up to 45 mg/kg/dose.

WVE-N531 demonstrated suitable pharmacokinetics profiles, including plasma protein binding, tissue distribution, metabolism, etc., for administration to human subjects in pharmacokinetic studies conducted in animals including mice and monkeys.

Non-GLP and GLP repeat-dose toxicity studies have been conducted including in CD-1 mice (up to 14 weeks) and cynomolgus monkeys (up to 14 weeks) using the IV route of administration. WVE-N531 has also been evaluated for potential genotoxicity in 2 in vitro tests and an in vivo study in mice. All GLP studies fulfilled the Organisation for Economic Cooperation and Development (OECD) mutual acceptance of data criterion [OECD Decision Reference C(89)87(Final)].

One nonclinical program evaluating WVE-N531 includes non-GLP repeat-dose toxicity studies in both mice and monkeys, and 14-week repeat-dose GLP toxicity studies with 13-week recovery in both species, in vitro and in vivo genetic toxicology, and in vivo safety pharmacology.

Mouse: In male mice, following 6 weeks of WVE-N531 administration via weekly IV bolus injection (0, 30, 90, or 180 mg/kg/dose), some inflammatory effects were observed. Overall, WVE-N531 was considered well tolerated in mice at doses up to 180 mg/kg. Repeat administration of WVE-N531 (0, 15, 60, or 180 mg/kg/dose) administered every other week for 14 weeks via IV bolus injection (a total of 8 doses) in male and female CD-1 mice was evaluated. NOAEL for this study was considered to be 60 mg/kg/dose. This dose level corresponded to mean sex-combined Cₘₐₓ and AUCₗₐₛₜ value of 1002±135 μg/mL and 1330±109 μg·h/mL, respectively, on Day 99 of the dosing phase.

Monkey: In male monkeys, WVE-N531 was considered well tolerated up to dose levels of 25 mg/kg administered once weekly and 45 mg/kg administered every other week. In view of the mild severity of findings, infrequent incidence and/or the lack of impact on the health and well-being of animals administered 45 mg/kg/dose, effects for this dose were considered nonadverse. NOAEL for this study was considered to be the 45 mg/kg/dose. The dose level of 45 mg/kg/dose (administered every other week) corresponded to Cₘₐₓ and AUCₗₐₛₜ values of 529 g/mL and 4380 g·hr/mL, respectively, on Day 29 of the dosing phase.

Results of various nonclinical studies show that the primary findings associated with WVE-N531 in mice and monkeys are consistent with class effects of oligonucleotides, including some potential for systemic inflammatory and coagulation effects, decreased cellularity of the thymus (mice only), as well as effects in the liver and kidney. No effects were observed on cardiovascular or respiratory function following a 60-minute IV infusion of WVE-N531 administered to male cynomolgus monkeys up to 45 mg/kg/dose. WVE-N531 was not associated with genotoxicity, evaluated in standard in vitro and in vivo assays.

These findings among other things establishes that WVE-N531 is sufficiently safe for administration to human subjects according to various methods, including various doses and dosage regimens described herein.

### Example 2. WVE-N531 Provides Safe and Effective Exon Skipping in Primate

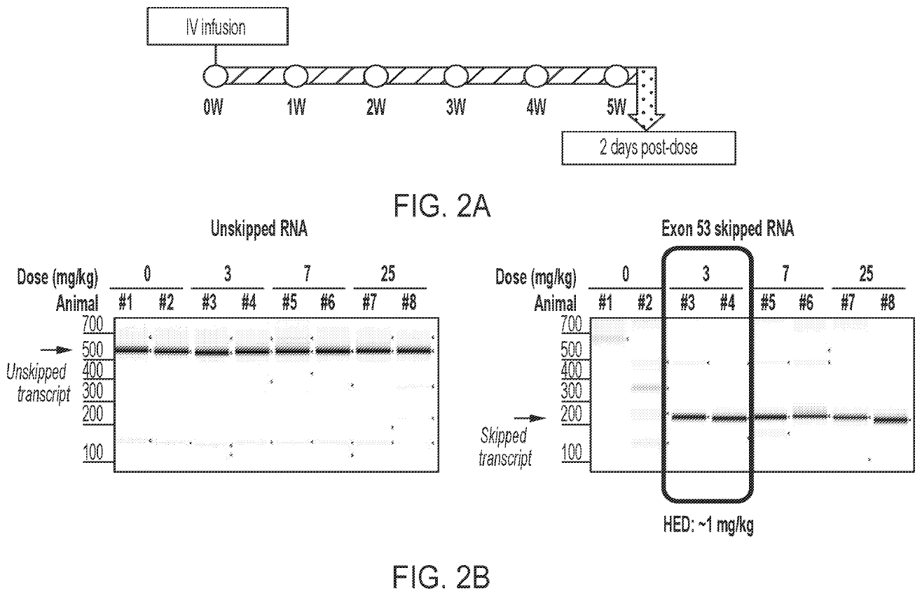

Among other things, the present disclosure demonstrates that WVE-N531 can provide effective exon skipping in non-human primates at various dosages. Monkeys were necropsied 2 days after the last of 6 weekly doses. To detect exon 53 skipping in DMD transcript, cDNA was prepared from RNA samples. Using a non-quantitative RT-PCR assay, exon 53 skipping was evaluated in monkeys receiving WVE-N531. As shown in FIG. 2, “unskipped” transcript was detected in all samples (Panel A, arrow showing band), while the “skipped” transcript was detected only in samples from WVE-N531 treated animals (Animals #3-#8; Panel B), demonstrating that exon skipping was achieved in all doses at 3 mg/kg and above.

Compared to suvodirsen (25 mg/kg twice a week), WVE-N531 (45 mg/kg every other week) provided higher plasma Cₘₐₓ (in some instances, about 2 to 3 fold), AUC (in some instances, about 8-13 fold) and Ctrough in non-human primate animals. WVE-N531 concentrations in muscles including heart and diaphragm were substantially higher, in various instances several fold higher, than suvodirsen in non-human primate animals.

### Example 3. Clinical Studies of WVE-N531

### Safety and Efficacy of WVE-N531 are Assessed in Clinical Trials.

A clinical study with WVE-N531 is a Phase 1b/2a open-label study to evaluate the safety, tolerability, PK, PD, and clinical effects of IV WVE-N531 in patients with DMD with a mutation that is amenable to exon 53 skipping intervention.

1 mg/kg (unless otherwise specified, dose is equivalent to WVE-N531 free acid form) was shown to be safe in human subjects.

One or more of various higher doses, e.g., about 2, 3, 4, 5, 6, 7, 8, 9, 10, 11, 12, 13, 14, 15, 16, 17, 18, 19, or 20 mg/kg, are administered through a dose escalation study, and safety profiles and effects (e.g., DMD exon 53 skipping, DMD protein restoration, biological function restoration, etc.) of various doses and dosage regimens are assessed. In some embodiments, a dose or each dose is independently about 3 mg/kg. In some embodiments, a dose or each dose is independently about 4 mg/kg. In some embodiments, a dose or each dose is independently about 5 mg/kg. In some embodiments, a dose or each dose is independently about 6 mg/kg. In some embodiments, a dose or each dose is independently about 7 mg/kg. In some embodiments, a dose or each dose is independently about 8 mg/kg. In some embodiments, a dose or each dose is independently about 9 mg/kg. In some embodiments, a dose or each dose is independently about 10 mg/kg. In some embodiments, a dose or each dose is independently about 11 mg/kg. In some embodiments, a dose or each dose is independently about 12 mg/kg. In some embodiments, a dose or each dose is independently about 13 mg/kg. In some embodiments, a dose or each dose is independently about 14 mg/kg. In some embodiments, a dose or each dose is independently about 15 mg/kg. In some embodiments, a dose or each dose is independently about 16 mg/kg. In some embodiments, a dose or each dose is independently about 17 mg/kg. In some embodiments, a dose or each dose is independently about 18 mg/kg. In some embodiments, a dose or each dose is independently about 19 mg/kg. In some embodiments, a dose or each dose is independently about 20 mg/kg. In some embodiments, about 1 mg/kg is administered. In some embodiments, about 3 mg/kg is administered. In some embodiments, about 5 mg/kg is administered. In some embodiments, about 8 mg/kg is administered. In some embodiments, about 10 mg/kg is administered. In some embodiments, about 15 mg/kg is administered. In some embodiments, about 20 mg/kg is administered. In some embodiments, about 3-5 mg/kg is administered. In some embodiments, about 5-10 mg/kg is administered. In some embodiments, about 10-15 mg/kg is administered. In some embodiments, about 15-20 mg/kg is administered. In some embodiments, a dosage regimen comprises or consists of one or more doses, each independently about 3-20 mg/kg, e.g., about 3-5, 5-10, 10-15, 15-20, or about 3, 4, 5, 6, 7, 8, 9, 10, 11, 12, 13, 14, 15, 16, 17, 18, 19, or 20 mg/kg, and each independently administered about 1, 2, 3, 4, 5, 6, 7, 8, 9, or 10 weeks, or about 1, 2, 3, 4, 5, 6, 7, 8, 9, 10, 11 or 12 months after its immediate preceding dose (if two or more doses are administered). In some embodiments, each dose in a dosage regimen is about the same. In some embodiments, two or more or all intervals between two consecutive doses are the same. In some embodiments, one or more later doses may be administered with longer intervals compared to the initial or earlier doses.

In some embodiments, a clinical trial is or comprises one or more conditions, protocols, methods, criteria, out measures, designs, etc. of NCT04906460. In some embodiments, a clinical trial is NCT04906460.

A useful clinical study protocol is presented below as an example. In some embodiments, a clinical trial is or comprises one or more conditions, protocols, methods, criteria, out measures, designs, etc. of the study described below. In some embodiments, a clinical trial is conducted according to the study described below. Those skilled in the art appreciate that the study may be adjusted.

An Open-Label Phase 1b/2a Study of WVE-N531 in Patients with Duchenne Muscular Dystrophy

Among other things, a study assess one or more of safety and tolerability of WVE-N531 in patients with Duchenne muscular dystrophy (DMD) amenable to exon 53 skipping intervention, concentration of WVE-N531 in muscle tissue, pharmacodynamic (PD) effect of WVE-N531, e.g., by assessing changes in dystrophin levels in deltoid muscle tissue, pharmacokinetics (PK) of WVE-N531, and clinical effects of WVE-N531. e.g., by assessing changes in muscle function, muscle strength, and pulmonary function.

To participate in the study, patients must have a documented mutation of the DMD gene that is amenable to exon 53 skipping intervention.

This study has 2 parts. Patients in Part A (up to 5 patients) will receive ascending doses of WVE-N531 followed by a ≥28-day safety monitoring period between each dose, starting at the lowest dose level. The Sponsor plans to evaluate 4 dose levels to carefully assess acute tolerability with ascending doses in an effort to titrate to a dose level optimized for PD response. The number of dose levels will be determined based on ongoing review of the safety and PK data as will a dose level for multiple-dose treatment in Part B of the study. Approximately 12 patients in Part B will receive 7 doses administered every other week at the selected dose level. Patients in Part A may receive up to an additional 3 doses of WVE-N531 administered every other week at a fixed dose level provided that they did not experience any safety events that would preclude further participation in the study. Patients in Part A will receive a maximum of 7 total doses. All dosing recommendations will be reviewed and endorsed by an independent Data Safety Monitoring Board (DSMB), with input from site Investigators.

Patients in Part A will undergo a biopsy (from deltoid) in the follow-up period after having received 7 doses. Patients in Part B will undergo a baseline biopsy and a biopsy in the follow-up period after completing dosing.

Criteria for dose selection in Part A and the decision to initiate Part B are detailed below.

### Part A.

Patients will initiate screening up to 6 weeks prior to starting in Part A. Part A will have an intra-patient dose escalation portion and a multiple-dose portion. A minimum of 3 patients will be enrolled and will receive a maximum of 7 doses of WVE-N531. Up to 5 patients may be enrolled.
Four ascending doses of WVE-N531 will be administered with a ≥28-day safety monitoring period between each dose.
Up to 3 additional biweekly doses at a fixed dose level.

The first dose level evaluated is 1 mg/kg. Dosing in Part A at all dose levels will be performed in a staggered manner. No more than 1 patient will be dosed per 48 hours during Part A. All patients will stay in the clinic through a 24-hour postdose monitoring period and return on Day 3 (approximately 48 hours postdose) for a safety follow-up. The decision to proceed with the dosing of the next patient in the same dose level will be based on the review of the safety data of the 48-hour observation period of the previous patient dosed, and the decision to proceed to the next patient will take into consideration the Investigator's assessment. After at least 3 patients complete the 2-week postdose follow-up visit at a dose level, all available safety and PK data will be reviewed. The recommended dose level for the subsequent dose administration will be reviewed and endorsed by the DSMB, with input from site Investigators. Patients will continue to be assessed for safety, PK, and functional assessments for a minimum of 28 days postdose for each dose level evaluated, prior to receiving the next dose level. Any additional patients enrolled after a DSMB review will receive the subsequent dose level selected for evaluation. Details regarding DSMB review are provided below.

Once the maximum tolerated dose (MTD) has been attained, patients in Part A will receive up to 3 additional biweekly doses of WVE-N531 at a fixed dose level (e.g., 10 mg/kg) provided that they did not experience any safety events that would preclude further participation in the study. Patients will receive a maximum of 7 doses in Part A of the study. Patients will undergo safety and PK assessments throughout the treatment period. Patients will undergo an open muscle biopsy 2 weeks after completion of multiple-dose treatment. Patients will continue to be monitored for safety through 10 weeks after the last dose. In some embodiments, ascending intra-patient single doses include or are about 1 mg/kg, about 3 mg/kg, about 6 mg/kg and about 10 mg/kg. In some embodiments, 3 additional doses of about 10 mg/kg, every other week, are administered.

Based on review of safety, PK, and muscle tissue concentrations of WVE-N531, a determination will be made about the initiation of Part B.

### Part B.

In Part B, approximately 12 new patients will be enrolled. Patients will initiate screening up to 6 weeks prior to dosing. Once patient eligibility has been determined, they will undergo a baseline open muscle biopsy. The baseline biopsy should occur at least 2 weeks prior to the subsequent first dose. Patients in Part B will receive 7 biweekly doses of WVE-N531. All patients will be assessed for safety, PK, PD, and clinical effects of WVE-N531 in Part B. In addition, patients will undergo regular functional assessments, including North Star Ambulatory Assessment (NSAA) 2.0, Performance of the Upper Limb (PUL) 2.0, four-stair climb, handheld myometry, and pulmonary function tests. These assessments should be performed in the order specified below. Functional assessments can be performed up to 24 hours predose, and should be performed before any other assessments that day, in particular blood draws. Patients will undergo a second open muscle biopsy 2 weeks after administration of the last dose. Patients will continue to be monitored for safety through 10 weeks after the last dose.

Dose selection will be primarily guided by clinical safety, tolerability, and PK data. In addition, the projected exposure at the highest dose to be tested is proposed not to exceed the lowest area under the plasma concentration-time curve from time 0 to the last quantifiable concentration (AUCₗₐₛₜ) value of 1330 μg-h/mL observed at the no-observed-adverse effect-level (NOAEL) of the mouse 14-week Good Laboratory Practice (GLP) toxicity study (60 mg/kg/dose) or the lowest maximum observed concentration (Cₘₐₓ) value of 852 μg/mL observed at the NOAEL of the monkey 14-week GLP toxicity study (45 mg/kg/dose).

The increase in dose for each dose level is no greater than 3-fold from the previous dose level.

The same dose level from the multiple-dose portion of Part A will be evaluated in Part B; however, a different dose level may be selected based on findings from Part A. The dose used in Part B will not exceed the MTD determined in Part A.

Number of Patients: Approximately 15 patients.

Study Population: Patients will be enrolled only if they qualify according to all of the following inclusion and exclusion criteria.

### Inclusion Criteria:

1. Patient and/or parent or legal guardian must have the ability and be willing to provide written informed consent prior to any study-related procedures.
2. Diagnosis of DMD based on clinical phenotype with increased serum creatine kinase.
3. Documented mutation in the DMD gene associated with DMD that is amenable to exon 53 intervention.
4. Score of ≥1 on item 1 or 2 of the shoulder component of the PUL.
5. Ambulatory or non-ambulatory male.
6. Age of ≥5 and ≤18 years at time of screening.
7. Willing and able to comply with scheduled visits, drug administration plan, laboratory tests, study restrictions, and all study procedures, including undergoing the muscle biopsy procedures.
8. Stable pulmonary and cardiac function, as measured by the following:
   a. Reproducible percent predicted forced vital capacity (FVC) ≥50%;
   b. Left ventricular ejection fraction (LVEF) >55% in patients <10 years of age and >45% in patients ≥10 years of age, as measured (and documented) by echocardiogram (ECHO) or cardiac magnetic resonance imaging (MRI).
9. Adequate deltoid muscle at Screening to perform open muscle biopsies.
10. Currently on a stable corticosteroid therapy regimen, defined as initiation of systemic corticosteroid therapy that occurred ≥6 months prior to Screening, and no changes in dose ≤3 months prior to Screening visit.
11. Sexually mature males must be willing to use contraception for the duration of the study and for 10 weeks (i.e., 5 half-lives) after last dose of study drug, if the patient is sexually active.
12. Patient and caregivers must agree not to post any study-related information on social media.

### Exclusion Criteria:

1. Clinically significant medical finding on the physical examination other than DMD that, in the judgment of the Investigator, will make the patient unsuitable for participation in, and/or completion of the study procedures.
2. Other prior or ongoing medical conditions that include the following:
   a. Acute illness within 4 weeks of the initial Screening visit;
   b. Abnormal physical findings, other than those associated with musculoskeletal findings attributable to DMD.
3. Laboratory abnormality, that, in the Investigator's opinion, could adversely affect the safety of the patient, make it unlikely that the course of treatment or follow-up would be completed, or impair the assessment of study results. These include the following, but are not limited to:
   a. Abnormal renal function, as defined by elevated serum Cystatin C level >1.5× the upper limit of normal (ULN) at Screening visit;
   b. Impaired hepatic function glutamate dehydrogenase (GLDH) 22.5×ULN and bilirubin 22×ULN (or international normalized ratio [INR]1.5×ULN);
   c. Activated partial thromboplastin time (aPTT) values 1.5×ULN;
   d. Platelet count below the lower limit of normal (LLN).
4. Documented positive hepatitis B surface antigen or hepatitis C antibody test.
5. Known to be positive for human immunodeficiency virus (HIV).
6. Severe cognitive disability and/or behavioral problems that, in the opinion of the Investigator, could prohibit participation in this study.
7. Cardiac insufficiency:
   a. Severe cardiomyopathy that, in the opinion of the Investigator, prohibits participation in this study; however, cardiomyopathy that is managed by angiotensin-converting-enzyme (ACE) inhibitors or beta blockers is acceptable provided the patient meets the LVEF inclusion criterion.
   b. Any other evidence of clinically significant structural or functional heart abnormality.
8. Need for daytime mechanical or noninvasive ventilation OR anticipated need for daytime mechanical or non-invasive ventilation within the next year, in the opinion of the Investigator. Nighttime non-invasive ventilation is permitted.
9. Changes in nutritional or herbal supplements or concomitant medications within 1 month prior to Screening visit or plans to modify (dose or regimen) during the study.
10. Currently on anticoagulants or drugs that are known to significantly increase the risk of bleeding, such as chronic non-steroidal anti-inflammatory drugs (NSAIDs) and heparin.
11. Received prior treatment with an investigational peptide-conjugated phosphorodiamidate morpholino oligomer (PPMO) or drisapersen.
12. Received prior treatment with gene therapy for DMD.
13. Received treatment with ataluren, viltolarsen, eteplirsen, or golodirsen within the 14 weeks prior to Screening.
14. Received any investigational drug within 3 months or 5 half-lives, whichever is longer prior to Screening.
15. Known hypersensitivity to any oligonucleotide, as demonstrated by a systemic allergic reaction such as changes in pulse, blood pressure, breathing function, etc.
16. Parent or legal guardian is directly or indirectly involved in the conduct and administration of this study as an Investigator, sub-investigator, study coordinator, or other study staff member; or the patient is a first-degree family member, significant other, or relative residing with one of the above persons involved directly or indirectly in the study.

Investigational Product, Dose, Route, Regimen: WVE-N531 will be provided as an isotonic solution for dilution for infusion. Four dose levels of WVE-N531 are planned. The route of administration will be IV. The infusion should be administered over a minimum of 60 minutes; however, the infusion time may be extended to a maximum of 3 hours if necessary to enhance tolerability.

Study Duration: Part A contains escalating doses with a ≥28-day safety monitoring period between each dose. Following selection of the multiple-dose level, patients in Part A will then receive up to 3 additional biweekly doses with a subsequent 10-week follow-up after the last dose. Patients will receive a total of 7 doses. Part B will consist of 7 biweekly doses and a 10-week follow-up after the last dose.

Endpoints:

### Safety

AEs; physical examination findings, including vital signs, safety laboratory tests (hematology, chemistry, coagulation, urinalysis); and electrocardiograms (ECGs) will be monitored/collected to assess the safety and tolerability of WVE-N531.

### Pharmacokinetics and Pharmacodynamics

Dystrophin level (% normal dystrophin), as assessed by Western blot of muscle tissue following multiple doses of WVE-N531.
Concentration of WVE-N531 in muscle tissue.
PK of WVE-N531.

### Clinical Effects

NSAA (Version 2.0)
PUL (Version 2.0)
Lower limb motor function by timed function tests (including 10-meter walk/run time, four-stair climb time, and time to rise from the floor)
Upper limb proximal strength assessed by handheld myometer
Pulmonary function tests (peak flow rate [PFR], cough peak flow [CPF], and FVC)

### Statistical Methods:

No formal sample size determination was performed for Part A. A maximum of 5 patients was considered sufficient for a preliminary evaluation of safety, tolerability, and PK of ascending doses of WVE-N531.

Part B was designed to demonstrate the effect of WVE-N531 on dystrophin (% normal) compared to baseline. The sample size determination was based on the following assumptions:
One-sample t-test to test that the change from baseline in dystrophin is greater than 0.
A mean increase in the change from baseline in dystrophin of 5 (% of normal) or more.
One-sided significance level of 2.5%.
Standard deviation (SD) of change from baseline in dystrophin level of 4.5%.
Dropout rate or nonevaluability rate of 10%.

### Analysis Populations

The safety population will include all treated patients.

The dystrophin analysis population will be a subset of the safety population and include patients with a postbaseline dystrophin value.

The PK population will be a subset of the safety population and include subjects with evaluable PK data.

### Analysis of Safety

Evaluation of the safety of WVE-N531 will include summaries of TEAEs, TEAEs leading to treatment discontinuation, TEAEs by severity, TEAEs by relationship, and serious TEAEs. Changes in laboratory assessments will also be summarized.

### Analysis of Change in Dystrophin Level (% of Normal)

The change in dystrophin level (% of normal) from baseline will be analyzed using a 1-sample t-test.

### Certain Study Design

This is an open-label study to evaluate the safety, tolerability, PK, PD, and clinical effects of intravenous (IV) WVE-N531 in patients with DMD. To participate in the study, patients must have a documented mutation of the DMD gene that is amenable to exon 53 skipping intervention.

### Screening

The screening period allows determination of patient eligibility for the study. It will begin when the study informed consent is signed by the patient and/or parent or legal guardian (as appropriate). In addition, the patient may be required to provide assent (as applicable). Patients will initiate screening up to 6 weeks prior to dosing. Screening assessments can occur on multiple days, provided they are within the Screening period. The Investigator will determine whether patients meet eligibility criteria and will collect the demographic and medical data permitting full characterization of the patient.

Patients must have a documented mutation in the DMD gene amenable to exon 53 skipping in order to participate in this study. This assessment must have been performed using an accepted laboratory method for mutation analysis.

### Treatment

### Part A

Part A will consist of an intra-patient dose escalation portion and a multiple-dose portion. Three patients will be enrolled and will receive a maximum of 7 doses of WVE-N531.

### Dose Escalation Portion

In the intra-patient dose escalation portion, patients will receive ascending doses of WVE-N531 with a ≥28-day safety monitoring period between each dose, starting at the lowest dose level. The first dose level evaluated will be 1 mg/kg. Dosing in Part A at all dose levels will be performed in a staggered manner. No more than 1 patient will be dosed per 48 hours during Part A. All patients will stay in the clinic through a 24-hour postdose monitoring period and return on Day 3/Visit 4 (approximately 48 hours postdose) for a safety follow-up. The decision to proceed with the dosing of the next patient in the same dose level will be based on the review of the safety data of the 48-hour observation period of the previous patient dosed, and the decision to proceed to the next patient will take into consideration the Investigator's assessment. Patients will return to the clinic for weekly visits through Day 22/Visit 7. Subsequent weekly safety monitoring will be completed by the Investigator via telephone/telemedicine visit until patients return for the next dose administration. There must be a minimum of 28 days between doses.

After a minimum of 3 patients completes the 2-week postdose follow-up visit at a dose level, all available safety and PK data will be reviewed. The recommended dose level for the subsequent dose administration will be reviewed and endorsed by the DSMB, with input from Investigators. Patients will continue to be assessed for safety, PK, and functional assessments for a minimum of 28 days postdose for each dose level evaluated, prior to receiving the next dose level. Any additional patients enrolled after a DSMB review will receive the subsequent dose level selected for evaluation.

### Multiple Dose Portion

Once the maximum tolerated dose (MTD) has been attained, patients in Part A will receive up to 3 additional biweekly doses of WVE-N531 at a fixed dose level provided that they did not experience any safety events that would preclude further participation in the study. All patients will receive a maximum of 7 total doses. Patients will undergo safety and PK assessments throughout the treatment period. Patients will undergo an open muscle biopsy from the deltoid muscle 2 weeks after completion of multiple-dose treatment. Patients will continue to be monitored for safety through up to 10 weeks after the last dose. Based on reviews of safety, PK, and muscle tissue concentrations of WVE-N531, a determination will be made about the initiation of Part B.

### Part B

In Part B, approximately 12 new patients will be enrolled. Patients in Part B will receive 7 biweekly doses of WVE-N531 at a fixed dose level and be assessed for safety, PK, PD, and clinical effects. Patients in Part B will undergo a baseline open muscle biopsy. This open muscle biopsy will be collected from the deltoid muscle and will be collected at least 2 weeks prior to the subsequent first dose (Part B Day 1/Visit 2) to allow for recovery from the surgery prior to obtaining baseline functional assessments. Patients will undergo a second open muscle biopsy 2 weeks after completion of the last dose. All patients will undergo regular assessments of the safety, PK, and PD of WVE-N531 throughout Part B. In addition, patients will undergo regular functional assessments, including NSAA 2.0, PUL 2.0, four-stair climb, handheld myometry, and pulmonary function tests. Functional assessments can be performed up to 24 hours predose and should be performed before any other assessments that day, in particular blood draws.

At the Part B Week 14/Visit 10 visit (a minimum of 2 weeks after the last dose), all patients will undergo a second muscle biopsy to evaluate the PD effects of WVE-N531 on muscle tissue. In addition, the concentrations of WVE-N531 in muscle tissue will be evaluated. All patients in Part B will be followed through Week 22/Visit 13 (10 weeks after the last dose) for safety.

If a patient withdraws from the study early, the patient should complete an early termination (ET) visit. Study Endpoints

### Safety

Adverse events (AEs) and physical examination findings including vital signs, safety laboratory tests (hematology, chemistry, coagulation, urinalysis), and electrocardiograms (ECGs) will be monitored/collected to assess the safety and tolerability of WVE-N531.

### Pharmacokinetics and Pharmacodynamics

Dystrophin level (% normal dystrophin) as assessed by Western blot of muscle tissue following multiple doses of WVE-N531
Concentration of WVE-N531 in muscle tissue
PK of WVE-N531

### Clinical Effects

NSAA (Version 2.0)
PUL (Version 2.0)
Lower limb motor function by timed function tests (including 10-meter walk/run time, four-stair climb time, and time to rise from the floor)
Upper limb proximal strength assessed by handheld myometer
Pulmonary function tests (peak flow rate [PFR], cough peak flow [CPF], and FVC)

WVE-N531 will be provided as an isotonic solution for dilution for IV infusion. WVE-N531 is supplied as a solution in a single-use vial. Study drug should be diluted with 0.45% sodium chloride injection or 0.9% sodium chloride injection prior to infusion in accordance with instructions. Study drug will be administered at the study site by trained personnel. The route of administration will be IV and the total volume of the infusion will be 100 mL to 500 mL based on patient weight. The infusion should be administered over a minimum of 60 minutes. However, the infusion time may be extended to a maximum of 3 hours if necessary to potentially enhance tolerability. The prepared infusion solution contains no preservatives and should be administered within 4 hours. WVE-N531 is reconstituted/diluted in the saline infusion bag/container. Patients should remain stationary during the infusion.

Treatment Compliance: As this is an IV administered drug that will be dosed in the clinic, treatment noncompliance is not expected to be an issue.

### Methods of Assessment and Endpoints

Safety Assessments. The safety assessments will include the following:
AEs
Medical history and demographics
Prior and concomitant medications
Physical examinations (including neurological and psychiatric)
Vital signs
Height and weight
12-lead ECG
ECHO or cardiac MRI
Clinical laboratory evaluations (including clinical chemistry, hematology, and urinalysis)

Any abnormal laboratory test results (hematology, clinical chemistry, or urine) or other safety assessments (e.g., vital sign measurements) that are clinically significant in the medical and scientific judgment of the Investigator are to be recorded as AEs or SAEs.

### Pharmacokinetic Assessments

Plasma Pharmacokinetics: Sampling for all patients will be collected per the schedule above. Blood samples will be collected for analysis done by a Sponsor-approved CRO. Samples will only be used by the Sponsor and/or a contracted vendor for research related to the development of treatments for DMD and will be stored for a maximum of 15 years. All biological material will be stored and secured in a way that ensures that unauthorized access is prohibited and the samples are not lost, deteriorated, or destroyed accidentally or illegally. Detailed instructions for sample collection, storage, processing, and shipping will be provided in the study-specific manual.

Muscle Concentration of WVE-N531: WVE-N531 concentration in muscle tissue may be evaluated by hybridization-ligation enzyme-linked immunosorbent assay (HL-ELISA; if adequate tissue sample is available). Samples will only be used by the Sponsor and/or a contracted vendor for research related to the development of treatments for DMD and will be stored for a maximum of 15 years. All biological material will be stored and secured in a way that ensures that unauthorized access is prohibited and the samples are not lost, deteriorated, or destroyed accidentally or illegally. Detailed instructions for sample collection, storage, processing, and shipping will be provided in the study-specific manual.

Immunogenicity: Immunogenicity samples will be collected to assess anti-drug and anti-dystrophin antibodies in serum, according to the schedules of assessments. Samples will only be used by the Sponsor and/or a contracted vendor for research related to the development of treatments for DMD and will be stored for a maximum of 15 years. All biological material will be stored and secured in a way that ensures that unauthorized access is prohibited and the samples are not lost, deteriorated, or destroyed accidentally or illegally. Detailed instructions for sample collection, storage, processing, and shipping will be provided in the study-specific manual.

Pharmacodynamic Assessments: Tissue samples collected from muscle biopsies will be evaluated for changes in dystrophin protein level. In addition, exploratory immunohistochemistry assessments for the presence of drug or biomarkers may be performed. Serum samples for potential emerging biomarkers will also be collected at the time points noted in the schedules of assessments. Samples will only be used by the Sponsor and/or a contracted vendor for research related to the development of treatments for DMD and will be stored for a maximum of 15 years. All biological material will be stored and secured in a way that ensures that unauthorized access is prohibited and the samples are not lost, deteriorated, or destroyed accidentally or illegally.

Dystrophin Quantification by Western Blot: Levels of dystrophin may be quantified by Western blot in the muscle tissues collected at baseline (Part B) and at the end of treatment (Parts A and B). The amount of restored dystrophin protein from muscle tissues will be evaluated using a validated Western blot method.

Clinical Effects: All functional assessments must be performed predose (on the dosing day or up to 24 hours prior to respective dosing). Functional assessments that are included in both NSAA and Timed Function Tests will only be administered once per time point as part of the NSAA, and scored according to both assessments. Functional assessments should be performed in the order specified below:
NSAA 2.0
PUL 2.0
Four-stair Climb
Handheld myometry Pulmonary function tests (PFR, CPF, and FVC)

Measurements of motor function will be performed according to the schedule of assessments. Patients should only perform the motor function and strength tests if they are able. If patients are unable to complete the tests, this should be documented in the case report forms.

North Star Ambulatory Assessment—Functional Assessment: The NSAA is a validated unidimensional scale specifically designed for measuring motor function in ambulatory children with DMD. The scale is suitable for multicentric global studies and is widely used internationally as a relevant functional assessment for patients with DMD. NSAA includes 17 items ranging from standing (item 1) to running (item 17) that are necessary to remain functionally ambulant. The scale includes items assessing abilities that can be present in the early stages of the disease such as head raise and standing on heels and other activities such as hopping or running that are generally never fully achieved in untreated children with DMD. Each item is scored on a 3-point scale using the following simple criteria: 2—Normal; achieves goal without any assistance; 1—Modified method; achieves goal independent of physical assistance from another person; and 0—Unable to achieve independently. The total score is the sum of the individual item scores. The score can range from 0, if no activities can be achieved independently, to 34, if all the activities can be achieved without any assistance. The scale is generally completed in a maximum of 15 minutes. The NSAA also includes the following timed assessments of lower limb motor function: time to stand from a supine position and time to walk 10 meters. An additional timed function test that is not part of NSAA is time to climb 4 standard-sized stairs.

Performance of Upper Limb (PUL 2.0) Test: The PUL test is an assessment tool that evaluates upper limb function in ambulatory and non-ambulatory patients with DMD and was developed through a collaborative international group including boys with DMD and their families. The PUL test was designed with a conceptual framework reflecting the progression of weakness and the natural history of functional decline in DMD.

Four-stair Climb: The time to climb 4 standard-sized stairs will measure lower limb motor function in ambulatory patients.

Handheld Myometry: Upper limb proximal strength will be assessed by handheld myometry.

Pulmonary Function Tests: Pulmonary function tests (PFR, CPF, and FVC) will be performed using the Microlab Spirometer. PFR and CPF: Muscle weakness caused by neuromuscular disorders results in reduced values for PFR. This test is effort dependent. The use of CPF can minimize effort-related variation. Thus, for patients with neuromuscular weakness, CPF measured by peak flow meter is a reliable measurement of expiratory muscle strength. FVC: During the FVC assessment, the patient takes a maximum breath and fills his lungs to total lung capacity (TLC) and then exhales to the maximum. The total volume of air expelled during forced exhalation after maximum inspiration is the FVC. The values are reduced in patients with neuromuscular disease. Measurements of FVC will be collected while the patient is in the sitting position. If the patient is wearing a thoracolumbar device, the measurements will be taken with the device on and the patient in a sitting position

### Statistical Methods

Study Drug Exposure and Compliance: The extent of study drug exposure and compliance will be summarized for the safety population.

### Analysis of Safety Data

Analyses will include Part A and Part B analyses. All safety analyses will be performed on the safety population using the following common rules:
The baseline value is defined generally as the last available value before first dose of study drug.
Adverse event observation periods are defined as follows:
   Pretreatment AEs are AEs that developed or worsened prior to the first dose of study drug.
   On-treatment AEs are AEs that developed or worsened from the first dose of study drug to study completion/discontinuation. Summaries of TEAEs will include all on-treatment AEs.
Quantitative safety parameters based on central laboratory/reading measurement descriptive statistics will be used to summarize results and change from baseline values by visit.

The amount of WVE-N531 present in plasma and muscle are assessed. Pharmacokinetic parameters include Cₘₐₓ (maximum observed concentration), AUCₗₐₛₜ (area under the plasma concentration-time curve from time 0 to the last quantifiable concentration), AUC_{inf} (area under the plasma concentration-time curve from time 0 to infinity), t_{1/2} (terminal half-life), CL (total body clearance) and Vdₛₛ (volume of distribution at steady state). In some embodiments, provided technologies provide improved pharmacokinetic properties compared to existing oligonucleotide therapeutics.

Immunogenicity of WVE-N531 is assessed, including antibodies against WVE-N531 and/or dystrophin. In some embodiments, provided technologies do not induce antibodies against WVE-N531 and/or dystrophin in serum, or levels of such antibodies are acceptable so that WVE-N531 can be administered to subjects.

In some embodiments, change in dystrophin level (% of normal) from baseline is assessed. In some embodiments, it is analyzed using a 1-sample t-test.

In some embodiments, an interim analysis is performed. In some embodiments, no interim analysis is performed.

In some embodiments, a study will be conducted according to the study protocol and SOPs that meet the guidelines provided by the International Conference on Harmonisation (ICH) for Good Clinical Practice (GCP) in clinical studies and any other applicable local regulatory requirements.

In a Phase 1b/2a open-label clinical trial, patients received a single dose of WVE-N531 and suvodirsen both at 1 mg/kg. Plasma AUC for WVE-N531 was about 4 fold of that of suvodirsen, and Cₘₐₓ for WVE-N531 was about 2.5 fold of that of suvodirsen. WVE-N531 demonstrated a much longer plasma half-life than suvodirsen: while plasma half-life for suvodirsen was less than 24 hours, half-life of WVE-N531 was estimated to be over 1 week.

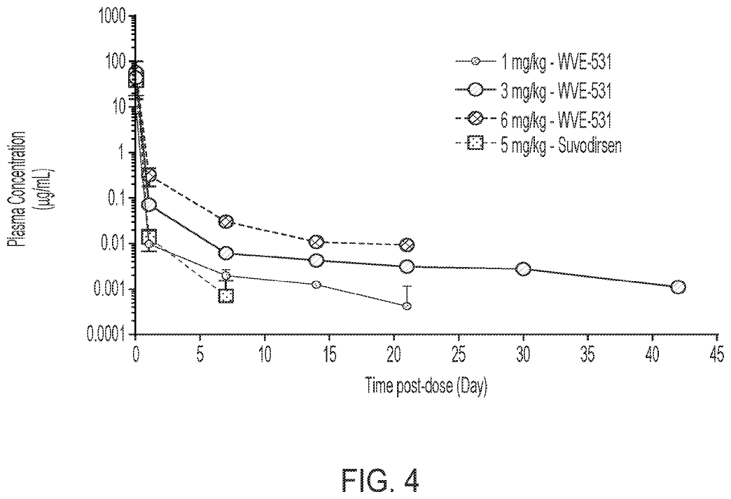

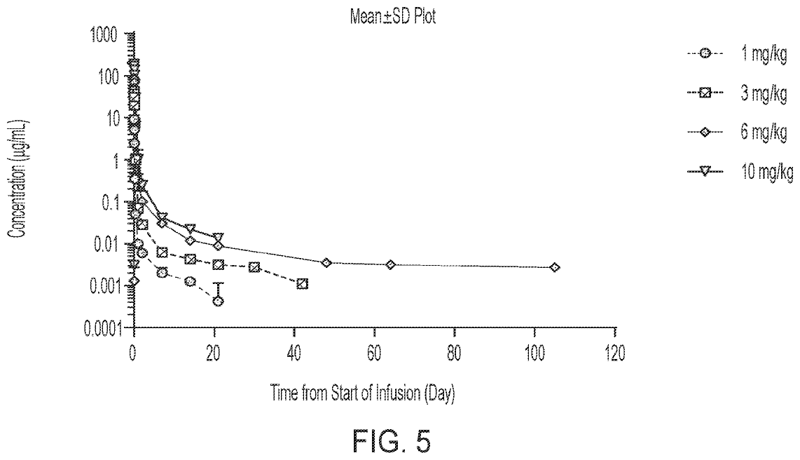

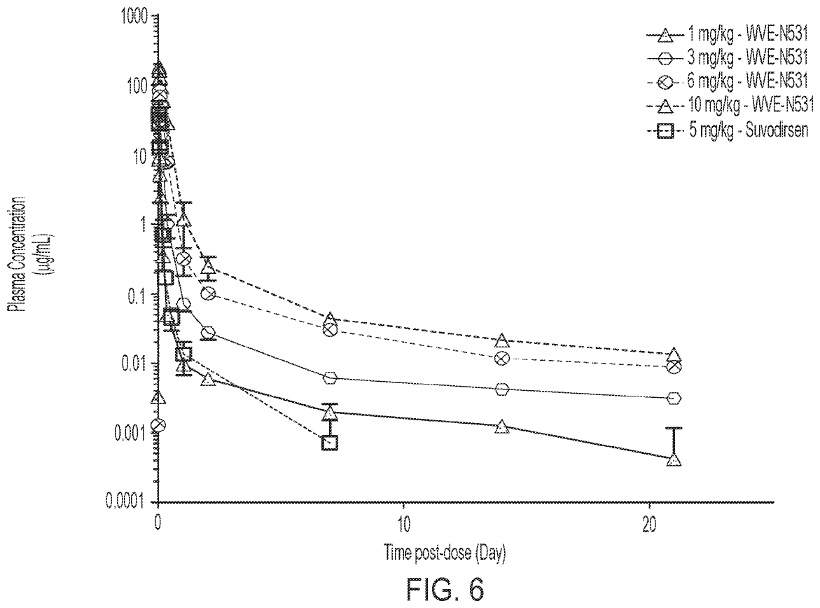

Additional data confirmed WVE-N531 can provide various advantages supporting its therapeutic uses described herein. In a Phase 1b/2a open-label clinical trial, patients received a single dose of 1 mg/kg, 3 mg/kg or 6 mg/kg of WVE-N531, or a single dose of 1 mg/kg, 5 mg/kg, 7 mg/kg, or 10 mg/kg of suvodirsen. As depicted in FIG. 4 and FIG. 6 and Table 1 below, plasma AUCₗₐₛₜ for 6 mg/kg WVE-N531 was about 4.6 fold of that of 5 mg/kg suvodirsen, and plasma Cₘₐₓ for 6 mg/kg WVE-N531 was about 2.1 fold of that of 5 mg/kg suvodirsen. Plasma concentration for 6 mg/kg WVE-N531 was about 43 fold of that of 5 mg/kg suvodirsen at 7 days. As depicted in FIG. 5, a single dose of 6 mg/kg WVE-N531 can provide quantifiable plasma concentration (e.g., >0.002 g/mL) at at least 105 days post-infusion. Further, as seen in FIG. 6 and Table 1 below, plasma AUCₗₐₛₜ for 10 mg/kg WVE-N531 was about 11 fold of that of 5 mg/kg suvodirsen and plasma concentration for 10 mg/kg WVE-N531 was about 58 fold of that of 5 mg/kg suvodirsen at 7 days. In some embodiments, plasma concentrations and other pharmacokinetic parameters for a single dose of 10 mg/kg WVE-N531 demonstrated a half-life of approximately 25 days, which may support monthly dosing. Even at lower doses, WVE-N531 can provide higher plasma concentration compared to suvodirsen (see, e.g., data for 3 mg/kg and 1 mg/kg WVE-N531 compared to those for 5 mg/kg or even 10 mg/kg suvodirsen).

TABLE 1
Clinical pharmacokinetic (PK) plasma data.

| | Dose | AUCₗₐₛₜ | | Cₘₐₓ | | Conc. @7 days | | Conc. @14 days | |
|---|---|---|---|---|---|---|---|---|---|
| Compound | (mg/kg) | (μg/mL*h) | | (μg/mL) | | (ng/mL) | | (ng/mL) | |
| WVE-N531 | 1 | 24.6 | (±1.83) | 9.60 | (±1.79) | 1.98 | (±0.649) | 1.25 | (±0.108) |
| | 3 | 150 | (±24.7) | 43.5 | (±6.81) | 6.15 | (±0.401) | 4.26 | (±0.152) |
| | 6 | 372 | (±37.4) | 85.6 | (±8.97) | 30.3 | (±5.53) | 10.8 | (±1.82) |
| | 10 | 926 | (±107) | 191 | (±18.1) | 41.3 | (±4.51) | 22.1 | (±2.76) |

| Suvodirsen | 1 | 5.90 | (±4.69) | 3.76 | (±2.88) | BLQ | | N/A |
|---|---|---|---|---|---|---|---|---|
| | 5 | 80.8 | (±43.4) | 40.8 | (±21.4) | 0.711 | (±0.739) | N/A |

| | | | | |
|---|---|---|---|---|
| 7 | 84.8 | 33.4 | BLQ | N/A |
| 10 | 354 | 147 | 4.10 | N/A |

BLQ = below limit of quantification

In a Phase 1b/2a open-label clinical trial, patients received single escalating doses of 1, 3, 6, and 10 mg/kg WVE-N531 and then, after about 1 to 2 months, received three doses of 10 mg/kg WVE-N531 every other week (week 0, week 2, week 4). At two weeks after the final dose (week 6), muscle biopsies were performed. Muscle biopsies were analyzed for WVE-N531 concentration, WVE-N531 localization, % exon 53 skipping, and dystrophin expression. Certain data for individual patients is shown below in Table 2. The mean WVE-N531 muscle concentration was approximately 42 μg/g (approximately 6.1 μM). High levels of exon 53 skipping were observed: the mean exon 53 skipping by RT-PCR was approximately 53%. To the Applicant's knowledge, it was the earliest time point as of Dec. 19, 2022, at which exon skipping had been reported in a clinical trial of boys with DMD. The mean dystrophin expression by Western blot was approximately 0.27% of normal (which is below the limit of quantification of 1%). Without the intention to be limited by theory, Applicant notes that protein production can lag behind splicing of RNA transcript. In some embodiments, WVE-N531 was visualized in myofiber nuclei using RNA in situ hybridization, confirming that WVE-N531 can reach the nucleus in muscle cells. Among other things, these data confirm that WVE-N531 can provide very high levels of exon 53 skipping, even at 10 mg/kg every two weeks which is significantly lower than golodirsen and/or viltolarsen.

TABLE 2
Clinical muscle biopsy data.

| | | Tissue | % Exon | Dystrophin by |
|---|---|---|---|---|
| | | Concentration | skipping | Western blot |
| Patient | Tissue Source | (μg/g) | by RT-PCR | (% of normal) |
| 1 | Deltoid | 85.5 | 61.5 | 0.24 |
| 2 | Deltoid | 33.5 | 49.8 | 0.23 |
| 3 | Bicep* | 8.3 | 47.9 | 0.34 |

*Biopsy was taken from bicep instead of deltoid muscle due to scarring of surrounding tissue from a prior biopsy.

WVE-N531 appeared safe and well-tolerated. Treatment-emergent adverse events (TEAEs) were mild, except for a single COVID-19 infection of moderate intensity. All adverse events (AEs) related to WVE-N531 (headache, pruritic rash) were mild, transient, and resolved without sequelae. There were no serious adverse events (SAEs), no events meeting stopping criteria, no trend for an increase in TEAEs, and no evidence of oligonucleotide class-related safety events (e.g., thrombocytopenia, coagulation, complement activation, cytokine activation).

Additional clinical trials can also be designed and conducted by those skilled in the art in accordance with the present disclosure to assess safety and efficacy of WVE-N531. For example, longer period of time may be utilized to assess and confirm production of dystrophin.

1. A method for treating muscular dystrophy, comprising administering to a subject suffering therefrom WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the subject has a mutation of the DMD gene that is amenable to exon 53 skipping.

2. A method, comprising administering to a subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for providing DMD exon 53 skipping in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for providing DMD exon 53 skipping in a subject, comprising administering to the subject one or more doses of WVE-N531, wherein each dose is independently equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for restoring DMD RNA reading frame in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for providing a DMD polypeptide in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the DMD polypeptide is truncated compared to a wild-type DMD protein; or
a method for providing increased level of a DMD function in a subject, comprising administering to the subject WVE-N531 at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

3. The method of claim 2, wherein the DMD polypeptide is encoded by an exon 53-skipped DMD mRNA and/or wherein the DMD polypeptide provides one or more functions of a wild-type DMD protein.

4. The method of any claim 2, wherein the subject has a mutation in the DMD gene that is amenable to exon 53 skipping and/or the subject is suffering from a muscular dystrophy, optionally wherein the subject is suffering from DMD.

5. The method of any one of claims 1-4, wherein WVE-N531 is administered in one or more forms, optionally one or more pharmaceutically acceptable salt forms, optionally wherein one form is WVE-N531 hexadecasodium salt.

6. A method for treating muscular dystrophy, comprising administering to a subject suffering therefrom a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the subject has a mutation of the DMD gene that is amenable to exon 53 skipping.

7. A method, comprising administering to a subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for providing DMD exon 53 skipping in a subject, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for restoring DMD RNA reading frame, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form; or
a method for providing a DMD polypeptide in a subject, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form, wherein the DMD polypeptide is truncated compared to a wild-type DMD protein; or
a method for providing increased level of a DMD function in a subject, comprising administering to the subject a pharmaceutical composition comprising WVE-N531 and a pharmaceutically acceptable carrier at a dose equivalent to about 1-20 (e.g., about 1-5, about 5-10, about 10-15, about 15-20, or about 1, 2, 3, 4, 5, 6, 7, 8, 9, 10, 11, 12, 13, 14, 15, 16, 17, 18, 19, or 20) mg/kg WVE-N531 free acid form.

8. The method of claim 7, wherein the DMD polypeptide is encoded by an exon 53-skipped DMD mRNA and/or wherein the DMD polypeptide provides one or more functions of a wild-type DMD protein.

9. The method of any one of claims 7-8, wherein the subject has a mutation in the DMD gene that is amenable to exon 53 skipping and/or the subject is suffering from a muscular dystrophy, optionally wherein the subject is suffering from DMD.

10. The method of any one of the preceding claims, wherein WVE-N531 in a dose is equivalent to about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg WVE-N531 free acid form.

11. The method of any one of the preceding claims, wherein two or more (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more) doses are administered.

12. The method of any one of claim 11, wherein each dose is independently administered in a pharmaceutical composition comprising WVE-N531 and a pharmaceutically acceptable carrier.

13. The method of any one of claims 11-12, wherein each dose has about the same amount of WVE-N531, optionally wherein WVE-N531 in each dose is equivalent to about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg WVE-N531 free acid form.

14. The method of any one of the preceding claims, wherein about is ±1%, ±2%, ±3%, ±4%, ±5%, ±6%, ±7%, ±8%, ±9%, or ±10%.

15. The method of any one of claims 6-14, wherein WVE-N531 exists in the pharmaceutical composition as one or more pharmaceutically acceptable salt forms, optionally wherein a pharmaceutically acceptable salt form is hexadecasodium salt.

16. The method of any one of claims 6-15, wherein the pharmaceutical composition is a liquid composition comprising dissolved WVE-N531.

17. The method of any one of claims 6-16, wherein the pharmaceutically acceptable carrier is or comprises a phosphate buffered solution.

18. The method of any one of claims 6-17, wherein the components in a pharmaceutical composition are WVE-N531, potassium phosphate monobasic, sodium phosphate dibasic, sodium chloride and water, and hydrochloric acid and/or sodium hydroxide for pH adjustment.

19. The method of any one of claims 6-18, wherein the pharmaceutical composition is isotonic.

20. The method of any one of claims 6-19, wherein the pharmaceutical composition has a pH of about 7-8, optionally wherein the pharmaceutical composition has a pH of about 7.3 or 7.4.

21. The method of any one of the preceding claims, wherein two, three, four, five, six, seven, eight, nine, ten or more consecutive doses are administered about weekly, or about every 2, 3, 4, 5, 6, 7, 8, 9, 10 weeks, or about every 1, 2, 3, 4, 5, 6, or more months.

22. The method of any one of the preceding claims, wherein all doses are administered about weekly, or about every 2, 3, 4, 5, 6, 7, 8, 9, 10 weeks, or about every 1, 2, 3, 4, 5, 6, or more months.

23. The method of claim 21 or 22, wherein each of the consecutive doses is independently equivalent to about 10 mg/kg WVE-N531 free acid form.

24. The method of any one of the preceding claims, wherein the composition has a purity of about 70%, 71%, 72%, 73%, 74%, 75%, 76%, 77%, 78%, 79%, 80%, 81%, 82%, 83%, 84%, 85%, 86%, 87%, 88%, 89%, 90% or more.

25. The method of claim 24, wherein the purity and/or impurities are measured by IP-RP-UPLC using area % at 260 nm, optionally using area % at 260 nm and the Set A parameters.

26. The method of any one of the preceding claims, wherein stereochemical purity is assessed by dimer modeling and/or wherein stereochemical purity of WVE-N531 is about 80%, 85%, 90% or more.

27. The method of any one of the preceding claims, wherein the amount of WVE-N531 is measured by UV at 260 nm and 27 OD/mg.

28. The method of any one of the preceding claims, wherein stereochemical identity of WVE-N531 is confirmed by IP-RP-UPLC, optionally according to Set B parameters.

29. The method of any one of the preceding claims, wherein a WVE-N531 drug substance is manufactured by a process described herein, characterized by one or more method described herein, released by one or more method described herein, and/or stored by one or more method described herein.

30. The method of claim 29, wherein the WVE-N531 drug substance is hexadecasodium salt.

31. The method of any one of the preceding claims, wherein a WVE-N531 drug product is manufactured by a process described herein, characterized by one or more method described herein, released by one or more method described, and/or stored by one or more method described herein.

32. The method of any one of the preceding claims, wherein a pharmaceutical composition is manufactured by a process described herein, characterized by one or more method described herein, released by one or more method described herein, and/or stored by one or more method described herein.

33. The method of any one of the preceding claims, wherein WVE-N531 is administered intravenously.

34. The method of any one of the preceding claims, wherein a DMD mutation is Δ3-52, Δ4-52, Δ5-52, Δ6-52, Δ9-52, Δ10-52, Δ11-52, Δ13-52, Δ14-52, Δ15-52, Δ16-52, Δ17-52, Δ19-52, Δ21-52, Δ23-52, Δ24-52, Δ25-52, Δ26-52, Δ27-52, Δ28-52, Δ29-52, Δ30-52, Δ31-52, Δ32-52, Δ33-52, Δ34-52, Δ35-52, Δ36-52, Δ37-52, Δ38-52, Δ39-52, Δ40-52, Δ41-52, Δ42-52, Δ43-52, Δ45-52, Δ47-52, Δ48-52, Δ49-52, Δ50-52, Δ51-52, Δ52, Δ54-58, Δ54-61, Δ54-63, Δ54-64, Δ54-66, Δ54-76, or Δ54-77, optionally wherein the DMD mutation comprises or is Δ52.

35. The method of any one of the preceding claims, wherein level of exon 53-skipped DMD mRNA is increased, or wherein about 10%, 15%, 20%, 25%, 30%, 35%, 40%, 45%, 50%, 55%, 60%, 65%, 70% or more of DMD mRNA is exon 53-skipped DMD mRNA after administration for a certain time period or after a certain number of doses.

36. The method of any one of the preceding claims, wherein a truncated DMD polypeptide is produced compared to a wild-type DMD protein, optionally wherein the truncated DMD polypeptide performs one or more functions of a wild-type DMD protein.

37. The method of any one of the preceding claims, wherein level of a truncated DMD polypeptide is increased, or wherein the method provide increases from baseline in dystrophin levels of about 1%, 2%, 3%, 4%, 5%, 5.3%, 6%, 7%, 8% 9%, 10%, 11%, 12%, 13%, 14%, 15%, 16%, 17%, 18%, 19%, 20% or more of normal levels after administration for a certain time period or after a certain number of doses.

38. The method of claim 37, wherein the increase is measured after administration for about 12 weeks, about 13 weeks, about 14 weeks, about 24 weeks, about 25 weeks, about 26 weeks, about 36 weeks, about 37 weeks, about 38 weeks, about 48 weeks, about 49 weeks, about 50 weeks, about 72 weeks, about 73 weeks, about 74 weeks, about 96 weeks, about 97 weeks, or about 98 weeks from first dose and/or wherein the increase is measured after 6, 7, 8, 9, 10, 11, 12, 13, 14, 15, 16, 17, 18, 19, 20, 21, 22, 23, 24, 25, 30, 35, 40, 45, 48, 49 or 50 doses.

39. The method of any one of the preceding claims, wherein a DMD function is increased and/or restored.

40. The method of any one of the preceding claims, wherein loss of ambulation in the subject is reduced, disease progression in the subject is delayed or slowed, muscle weakness in the subject is delayed or slowed, loss of muscle mass in the subject is delayed or slowed, and/or loss of pulmonary function in the subject is delayed or slowed, and/or wherein the subject improves in a muscular dystrophy assessment and/or one or more functional assessments, and/or wherein the subject improves in a 10 meter walk test, North Star Ambulatory Assessment (NSAA) 2.0, Performance of the Upper Limb (PUL) 2.0, four-stair climb, upper limb proximal strength, handheld myometry, time to rise from the floor, and/or one or more lower limb motor function by timed function tests, and/or wherein the subject improves in one or more pulmonary function tests, optionally wherein one or more pulmonary function tests are peak flow rate (PFR), cough peak flow (CPF), and/or FVC.

41. The method of claim 39 or 40, wherein an improvement is compared to baseline, absence of WVE-N531 administration, or administration of a reference composition, optionally wherein the reference composition is comparable to an administered WVE-N531 composition but does not contain WVE-N531.

42. The method of any of the previous claims, wherein the subject is a pediatric subject.

43. The method of any of the previous claims, wherein the subject is administered a steroid at least about one month, two months, three months, four months, five months, or six months prior to the first dose of WVE-N531, optionally wherein the steroid is a corticosteroid, optionally wherein the corticosteroid is deflazacort.

44. A composition comprising WVE-N531.

45. The composition of claim 44, wherein a form of WVE-N531 in the composition is a pharmaceutically acceptable salt form and/or WVE-N531 hexadecasodium salt.

46. The composition of any one of claims 44-45, wherein each form of WVE-N531 in the composition is independently a salt form, optionally a pharmaceutically acceptable salt form and/or WVE-N531 hexadecasodium salt.

47. The composition of any one of claims 44-46, wherein the composition is a drug substance and/or drug product.

48. The compound of any one of claims 44-47, wherein the composition is a liquid composition wherein WVE-N531 is dissolved.

49. The composition of any one of claims 44-48, wherein the composition is a pharmaceutical composition further comprising a pharmaceutically acceptable carrier, optionally wherein the pharmaceutically acceptable carrier is or comprises a phosphate buffered solution.

50. The composition of any one of claims 44-49, wherein the components in the composition are WVE-N531, potassium phosphate monobasic, sodium phosphate dibasic, sodium chloride and water, and hydrochloric acid and/or sodium hydroxide for pH adjustment, and/or wherein the composition is isotonic, and/or wherein the composition has a pH of about 7-8, optionally wherein the composition has a pH of about 7.3 or 7.4.

51. The composition of any one of claims 44-50, wherein the concentration of WVE-N531 is equivalent to about 5-45 mg/mL WVE-N531 free acid form, optionally wherein the concentration of WVE-N531 is equivalent to about 25-40 mg/mL WVE-N531 free acid form.

52. The composition of claim 51, wherein the concentration of WVE-N531 is equivalent to about 5-7 mg/mL WVE-N531 free acid form optionally wherein the concentration of WVE-N531 is equivalent to about 6 mg/mL WVE-N531 free acid form.

53. The composition of claim 52, wherein the composition is packaged into a vial, wherein the volume of the composition in the vial is at least 6-7 mL, at least 6-6.5 mL, or at least 6 mL.

54. The composition of any one of claims 44-53, wherein the composition has a purity of about 70%, 71%, 72%, 73%, 74%, 75%, 76%, 77%, 78%, 79%, 80%, 81%, 82%, 83%, 84%, 85%, 86%, 87%, 88%, 89%, 90% or more.

55. The composition of claim 54, wherein the purity and/or impurities are measured by an IP-RP-UPLC method for purity as described herein and/or IP-RP-UPLC using area % at 260 nm, optionally using area % at 260 nm and Set A parameters.

56. The composition of any one of claims 44-55, wherein stereochemical purity is assessed by dimer modeling and/or wherein stereochemical purity of WVE-N531 is about 80%, 85%, 90% or more.

57. The composition of any one of claims 44-56, wherein the amount of WVE-N531 is measured by UV at 260 nm, optionally at 260 nm and 27 OD/mg.

58. The composition of any one of claims 44-57, wherein the WVE-N531 drug substance in the composition is manufactured by a process described herein, characterized by one or more method described herein, released by one or more method described herein, and/or stored by one or more method described herein.

59. The composition of claim 58, wherein the WVE-N531 drug substance is hexadecasodium salt.

60. The composition of any one of claims 44-59, wherein the composition is a WVE-N531 drug product.

61. The composition of any one of claims 44-60, wherein WVE-N531 drug product is manufactured by a process described herein, characterized by one or more method described herein, released by one or more method described herein, and/or stored by one or more method described herein.

62. The composition of any one of claims 49-61, wherein a pharmaceutical composition is manufactured by a process described herein, characterized by one or more method described herein, released by one or more method described herein, and/or stored by one or more method described herein.

63. The composition of any one of claims 44-62, wherein the composition does not contain DS1, DS2, DS3, DS4, DS5, DS6, DS7, DS8, DS9, DS10, DS11, DS12, DS13, DS14, DS15, DS16, and/or DS17.

64. A composition comprising one of WVE-N531 and DS1 to DS17, wherein the composition is free of one or more (e.g., 1, 2, 3, 4, 5, 6, 7, 8, 9, 10, 11, 12, 13, 14, 15, 16 or 17) of the rest of WVE-N531 and DS1 to DS17.

65. The composition of claim 64, wherein the composition comprises WVE-N531.

66. The composition of claim 64, wherein the composition comprises DS1, DS2, DS3, DS4, DS5, DS6, DS7, DS8, DS9, DS10, DS11, DS12, DS13, DS14, DS15, DS16, or DS17.

67. A method for manufacturing a WVE-N531 composition according to a method described in the specification.

68. The method of claim 67, comprising utilizing IP-RP-UPLC to assess purity and/or impurities in the manufactured WVE-N531 composition and release the preparation if the purity and/or impurities meet certain criteria.

69. The method of any one of claims 67-67, wherein the composition is a drug substance or a drug product.

70. A method for releasing a WVE-N531 preparation, comprising utilizing IP-RP-UPLC to assess purity and/or impurities in the WVE-N531 preparation and release the preparation if the purity and/or impurities meet certain criteria; or
a method for assessing purity of WVE-N531 utilizing IP-RP-UPLC.

71. The method of claim 70, wherein the IP-RP-UPLC utilizes one or more parameters described in the specification and/or one or more parameters of Set A.

72. The method of claim 67-71, wherein stereochemical identity of WVE-N531 is confirmed by IP-RP-UPLC.

73. A method for confirming stereochemical identity of WVE-N531 utilizing IP-RP-UPLC.

74. The method of any one of claims 67-73, wherein stereochemical identity of WVE-N531 is confirmed by IP-RP-UPLC according to Set B parameters or an IP-RP-UPLC method for stereochemical identity as described herein.

75. The method of any one of claims 1-43 and 67-74, wherein the composition is any one of claims 44-66.

76. A compound, oligonucleotide, composition, method, process, use, dose or dosage regimen described in the specification or of any one of Example Embodiments 1-453.
